SEQUENCE LISTING (27 sequences)

SEQ ID NO: 1 - DNA, 2211 bp, Artificial Sequence
  AAVhu68 vp1 capsid
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 2 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - DNA, 2208 bp, Artificial Sequence
  AAV9 VP1 capsid
  CDS (1)..(2208): AAV9 VP1 Capsid
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 4 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - DNA, 2058 bp, Artificial sequence
  Human GALC coding sequence
  sig_peptide (1)..(126)
  CDS (1)..(2058)
atggctgagtggctactctcggcttcctggcaacgccgagcgaaagctatgactgcggccgcgggttcggcgggccgcgccgcggtgcccttgctgctgtgtgcgctgctggcgcccggcggcgcgtacgtgctcgacgactccgacgggctgggccgggagttcgacggcatcggcgcggtcagcggcggcggggcaacctcccgacttctagtaaattacccagagccctatcgttctcagatattggattatctctttaagccgaattttggtgcctctttgcatattttaaaagtggaaataggtggtgatgggcagacaacagacggcactgagccctcccacatgcattatgcactagatgagaattatttccgaggatacgagtggtggttgatgaaagaagctaagaagaggaatcccaatattacactcattgggttgccatggtcattccctggatggctgggaaaaggtttcgactggccttatgtcaatcttcagctgactgcctattatgtcgtgacctggattgtgggcgccaagcgttaccatgatttggacattgattatattggaatttggaatgagaggtcatataatgccaattatattaagatattaagaaaaatgctgaattatcaaggtctccagcgagtgaaaatcatagcaagtgataatctctgggagtccatctctgcatccatgctccttgatgccgaactcttcaaggtggttgatgttataggggctcattatcctggaacccattcagcaaaagatgcaaagttgactgggaagaagctttggtcttctgaagactttagcactttaaatagtgacatgggtgcaggctgctggggtcgcattttaaatcagaattatatcaatggctatatgacttccacaatcgcatggaatttagtggctagttactatgaacagttgccttatgggagatgcgggttgatgacggcccaggagccatggagtgggcactacgtggtagaatctcctgtctgggtatcagctcataccactcagtttactcaacctggctggtattacctgaagacagttggccatttagagaaaggaggaagctacgtagctctgactgatggcttagggaacctcaccatcatcattgaaaccatgagtcataaacattctaagtgcatacggccatttcttccttatttcaatgtgtcacaacaatttgccacctttgttcttaagggatcttttagtgaaataccagagctacaggtatggtataccaaacttggaaaaacatccgaaagatttctttttaagcagctggattctctatggctccttgacagcgatggcagtttcacactgagcctgcatgaagatgagctgttcacactcaccactctcaccactggtcgcaaaggcagctacccgcttcctccaaaatcccagcccttcccaagtacctataaggatgatttcaatgttgattacccattttttagtgaagctccaaactttgctgatcaaactggtgtatttgaatattttacaaatattgaagaccctggcgagcatcacttcacgctacgccaagttctcaaccagagacccattacgtgggctgccgatgcatccaacacaatcagtattataggagactacaactggaccaatctgactataaagtgtgatgtatacatagagacccctgacacaggaggtgtgttcattgcaggaagagtaaataaaggtggtattttgattagaagtgccagaggaattttcttctggatttttgcaaatggatcttacagggttacaggtgatttagctggatggattatatatgctttaggacgtgttgaagttacagcaaaaaaatggtatacactcacgttaactattaagggtcatttcacctctggcatgctgaatgacaagtctctgtggacagacatccctgtgaattttccaaagaatggctgggctgcaattggaactcactcctttgaatttgcacagtttgacaactttcttgtggaagccacacgctaa

SEQ ID NO: 6 - protein, 685 aa, Artificial sequence
  Synthetic Construct
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 7 - DNA, 2007 bp, Artificial sequence
  Engineered canine GALC
  CDS (1)..(2007)
atgacagccgctgctggatctgctggacacgctgctgttcctctgctgctgtgtgcactgctggtgcctggcggagcttacgtgctggatgattctgatggactaggccgcgagttcgacggcgtgggagctgtttctggtggcggagccacatctaggctgctggtcaattaccccgagccttacaggtcccagatcctggactacctgttcaagcccaacttcggcgccagcctgcacatcctgaaggtggaaattggcggcgacggccagaccaccgacggaacagaacctagccacatgcactacgccctggacgagaacttcttcaggggctacgagtggtggctgatgaaggaagccaagaagaggaaccccaacatcatcctgatgggcctgccttggtctttccctggctggatcggcaagggcttcaactggccttacgtgaacctgcagctgaccgcctactacatcatgacctggatcgtgggcgccaagcactaccacgacctggacatcgactacatcggcatctggaacgagcggagcttcgacatcaattacatcaaggtgctgcggcggatgctgaactaccagggcctcgacagagtgaagatcattgccagcgacaacctgtgggagcccatcagcgcttctatgctgctggactccgagctgctgaaagtgatcgacgtgatcggcgctcactaccccggaacacacaccgtgaaggacgccaaactgaccaagaagaagctgtggtccagcgaggacttcagcaccctgaatagtgacgtcggagccggctgcctgggcagaatcctgaaccagaactacgtgaacggctacatgaccgccacaatcgcctggaatctggtggccagctactacgagcagctgccctacggaaggtgcggcctgatgacagcccaagagccttggagcggacactacgtggtggaaagccctatctgggtgtcagcccacaccacacagttcacccagcctggctggtactacctgaaaaccgtgggccacctggaaaaaggcggctcttatgtggccctgaccgacggcctgggaaacctgacaatcatcgtggaaaccatgagccacaagcagagcgcctgcatcagaccctttctgccctacttcaacgtgtccaggcagttcgccaccttcgtgctgaagggcagcttcagcgagatccccgaactgcaagtgtggtacaccaagctgggcaagcccagcgagagatacctgtttaagcagctggacagcctgtggctgctcgacagcagctctaccttcacactggaactccaagaggacgagatcttcaccctgaccacactgaccgtgggcagcaagggatcttaccctctgcctcctaagagcgagccctttccacagatctacgaggacgacttcgacgtggactacccattctttagcgaggcccctaacttcgccgatcagaccggcgtgttcgagtacttcaccaacatcgaggaccccggcgagcacaggtttaccctgagacaggtgctgaaccagcggcctattacctgggccgctgacgcctacaacaccatctctatcatcggcgactacaagtggtccaacctgaccgtcagatgcgacgtgtacatcgagacacccgagaaaggcggagtgtttatcgccggcagagtgaacaaaggcggcatcctgatcagaagcgccaggggaatcttcttctggatcttcgccaacggcacctacagagtgacaggcgatctggccggatgggtcatctatgccctgggaagagtggacgtgaccgccaagaagtggtacactctgaccctgatcatcaagggcagactgagcagcggcatgctgaatggcaagaccgtgtggaagaacatccccgtgtctttccccaaaaacggctgggccgccatcggaacccacagcttcgaatttgcccagttcgacaactttcacgtggaagccaccaga

SEQ ID NO: 8 - protein, 669 aa, Artificial sequence
  Synthetic Construct
MTAAAGSAGHAAVPLLLCALLVPGGAYVLDDSDGLGREFDGVGAVSGGGATSRLLVNYPEPYRSQILDYLFKPNFGASLHILKVEIGGDGQTTDGTEPSHMHYALDENFFRGYEWWLMKEAKKRNPNIILMGLPWSFPGWIGKGFNWPYVNLQLTAYYIMTWIVGAKHYHDLDIDYIGIWNERSFDINYIKVLRRMLNYQGLDRVKIIASDNLWEPISASMLLDSELLKVIDVIGAHYPGTHTVKDAKLTKKKLWSSEDFSTLNSDVGAGCLGRILNQNYVNGYMTATIAWNLVASYYEQLPYGRCGLMTAQEPWSGHYVVESPIWVSAHTTQFTQPGWYYLKTVGHLEKGGSYVALTDGLGNLTIIVETMSHKQSACIRPFLPYFNVSRQFATFVLKGSFSEIPELQVWYTKLGKPSERYLFKQLDSLWLLDSSSTFTLELQEDEIFTLTTLTVGSKGSYPLPPKSEPFPQIYEDDFDVDYPFFSEAPNFADQTGVFEYFTNIEDPGEHRFTLRQVLNQRPITWAADAYNTISIIGDYKWSNLTVRCDVYIETPEKGGVFIAGRVNKGGILIRSARGIFFWIFANGTYRVTGDLAGWVIYALGRVDVTAKKWYTLTLIIKGRLSSGMLNGKTVWKNIPVSFPKNGWAAIGTHSFEFAQFDNFHVEATR

SEQ ID NO: 9 - DNA, 2055 bp, Artificial sequence
  Engineered human GALC coding sequence
  CDS (1)..(2055)
  sig_peptide (1)..(126)
  mat (127)..(2055)
atggccgagtggctgctgtctgctagctggcagagaagggccaaggccatgacagccgccgctggatctgcaggcagagctgctgtgcctctgctgctgtgtgcactgctggcacctggcggagcctacgtgctggatgattctgacggcctgggcagagagttcgacggcatcggagctgtgtctggcggcggagccacaagcagactgctcgtgaactaccccgagccctacagaagccagatcctggactacctgttcaagcccaacttcggcgccagcctgcacatcctgaaggtggaaatcggcggcgacggccagaccaccgatggcacagaacctagccacatgcactacgccctggacgagaactacttccggggctacgagtggtggctgatgaaggaagccaagaagcggaaccccaacatcaccctgatcggcctgccttggagcttccctggctggctgggcaagggcttcgactggccttacgtgaacctgcagctgaccgcctactacgtcgtgacctggatcgtgggcgccaagagataccacgacctggacatcgactacatcggcatctggaacgagcggagctacaacgccaactacatcaagatcctgcggaagatgctgaactaccagggcctgcagagagtgaagatcattgccagcgacaacctgtgggagagcatcagcgcctccatgctgctggacgccgagctgttcaaggtggtggatgtgatcggcgcccactaccctggcacacactctgccaaggacgccaagctgaccggcaagaagctgtggtccagcgaggacttcagcaccctgaacagcgacatgggagccggctgctggggcagaatcctgaaccagaattacatcaacggctacatgaccagcacaatcgcctggaacctggtggccagctactacgagcagctgccctacggcagatgcggcctgatgacagcccaggaaccttggagcggccactacgtggtggaaagccctgtgtgggtgtccgcccacaccacccagtttacacagcccggctggtactacctgaaaaccgtgggccacctggaaaagggcggcagctatgtggccctgacagacggactgggcaacctgaccatcatcatcgagacaatgtcccacaagcacagcaagtgcatcagaccctttctgccctacttcaacgtgtcccagcagttcgccaccttcgtgctgaagggcagcttcagcgagatccccgaactgcaagtgtggtacaccaagctgggaaagaccagcgagcggttcctgtttaagcagctggacagcctgtggctgctggacagcgacggcagctttaccctgagcctgcacgaggacgagctgtttacactgaccaccctgaccacaggccggaagggctcttaccccctgcctcctaagagccagcccttcccaagcacctacaaggacgacttcaatgtggactacccattctttagcgaggcccccaatttcgccgaccagaccggcgtgttcgagtacttcaccaacatcgaggaccccggcgagcaccacttcaccctgagacaggtgctgaaccagcggcccatcacctgggctgccgatgccagcaacaccatcagcatcatcggcgactacaactggaccaatctgacaatcaagtgcgacgtgtacatcgaaacccccgacaccggcggagtgtttatcgccggcagagtgaacaagggcgggatcctgatcagaagcgccagaggcatcttcttttggatcttcgccaacggcagctacagagtgaccggcgatctggccggctggatcatctacgctctgggcagggtggaagtgaccgccaagaaatggtacaccctgacactgactatcaagggccacttcgcctccggcatgctgaacgacaagagcctgtggaccgacatccccgtgaacttccccaagaatggctgggccgccatcggcacccacagctttgagttcgcccagttcgacaacttcctggtggaagccaccaga

SEQ ID NO: 10 - protein, 685 aa, Artificial sequence [sequence omitted]

SEQ ID NO: 11 - DNA, 130 bp, Artificial sequence
  AAV2 - 5′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 382 bp, Artificial sequence
  CMV IE enhancer
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 13 - DNA, 282 bp, Artificial sequence
  CB promoter
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 14 - DNA, 666 bp, Artificial sequence
  CB7 promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 15 - DNA, 972 bp, Artificial Sequence
  chimeric intron
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 16 - DNA, 127 bp, Artificial sequence
  Rabbit globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 17 - DNA, 130 bp, Artificial sequence
  AAV2 - 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 4337 bp, Artificial sequence
  Vector genome with canine GALC
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgacagccgctgctggatctgctggacacgctgctgttcctctgctgctgtgtgcactgctggtgcctggcggagcttacgtgctggatgattctgatggactaggccgcgagttcgacggcgtgggagctgtttctggtggcggagccacatctaggctgctggtcaattaccccgagccttacaggtcccagatcctggactacctgttcaagcccaacttcggcgccagcctgcacatcctgaaggtggaaattggcggcgacggccagaccaccgacggaacagaacctagccacatgcactacgccctggacgagaacttcttcaggggctacgagtggtggctgatgaaggaagccaagaagaggaaccccaacatcatcctgatgggcctgccttggtctttccctggctggatcggcaagggcttcaactggccttacgtgaacctgcagctgaccgcctactacatcatgacctggatcgtgggcgccaagcactaccacgacctggacatcgactacatcggcatctggaacgagcggagcttcgacatcaattacatcaaggtgctgcggcggatgctgaactaccagggcctcgacagagtgaagatcattgccagcgacaacctgtgggagcccatcagcgcttctatgctgctggactccgagctgctgaaagtgatcgacgtgatcggcgctcactaccccggaacacacaccgtgaaggacgccaaactgaccaagaagaagctgtggtccagcgaggacttcagcaccctgaatagtgacgtcggagccggctgcctgggcagaatcctgaaccagaactacgtgaacggctacatgaccgccacaatcgcctggaatctggtggccagctactacgagcagctgccctacggaaggtgcggcctgatgacagcccaagagccttggagcggacactacgtggtggaaagccctatctgggtgtcagcccacaccacacagttcacccagcctggctggtactacctgaaaaccgtgggccacctggaaaaaggcggctcttatgtggccctgaccgacggcctgggaaacctgacaatcatcgtggaaaccatgagccacaagcagagcgcctgcatcagaccctttctgccctacttcaacgtgtccaggcagttcgccaccttcgtgctgaagggcagcttcagcgagatccccgaactgcaagtgtggtacaccaagctgggcaagcccagcgagagatacctgtttaagcagctggacagcctgtggctgctcgacagcagctctaccttcacactggaactccaagaggacgagatcttcaccctgaccacactgaccgtgggcagcaagggatcttaccctctgcctcctaagagcgagccctttccacagatctacgaggacgacttcgacgtggactacccattctttagcgaggcccctaacttcgccgatcagaccggcgtgttcgagtacttcaccaacatcgaggaccccggcgagcacaggtttaccctgagacaggtgctgaaccagcggcctattacctgggccgctgacgcctacaacaccatctctatcatcggcgactacaagtggtccaacctgaccgtcagatgcgacgtgtacatcgagacacccgagaaaggcggagtgtttatcgccggcagagtgaacaaaggcggcatcctgatcagaagcgccaggggaatcttcttctggatcttcgccaacggcacctacagagtgacaggcgatctggccggatgggtcatctatgccctgggaagagtggacgtgaccgccaagaagtggtacactctgaccctgatcatcaagggcagactgagcagcggcatgctgaatggcaagaccgtgtggaagaacatccccgtgtctttccccaaaaacggctgggccgccatcggaacccacagcttcgaatttgcccagttcgacaactttcacgtggaagccaccagatgataacctcaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 4386 bp, Artificial sequence
  CB7.CI.hGALC.rBG
  misc_feature (1)..(130): 5′ ITR
  misc_feature (198)..(579): CMV IE Enhancer
  misc_feature (582)..(863): CB promoter
  misc_feature (836)..(839): TATA
  misc_feature (958)..(1930): chimeric intron
  misc_feature (1948)..(4002): hGALCco
  misc_feature (4042)..(4168): Rabbit globin poly A
  misc_feature (4257)..(4386): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcacgcgtgccaccatggccgagtggctgctgtctgctagctggcagagaagggccaaggccatgacagccgccgctggatctgcaggcagagctgctgtgcctctgctgctgtgtgcactgctggcacctggcggagcctacgtgctggatgattctgacggcctgggcagagagttcgacggcatcggagctgtgtctggcggcggagccacaagcagactgctcgtgaactaccccgagccctacagaagccagatcctggactacctgttcaagcccaacttcggcgccagcctgcacatcctgaaggtggaaatcggcggcgacggccagaccaccgatggcacagaacctagccacatgcactacgccctggacgagaactacttccggggctacgagtggtggctgatgaaggaagccaagaagcggaaccccaacatcaccctgatcggcctgccttggagcttccctggctggctgggcaagggcttcgactggccttacgtgaacctgcagctgaccgcctactacgtcgtgacctggatcgtgggcgccaagagataccacgacctggacatcgactacatcggcatctggaacgagcggagctacaacgccaactacatcaagatcctgcggaagatgctgaactaccagggcctgcagagagtgaagatcattgccagcgacaacctgtgggagagcatcagcgcctccatgctgctggacgccgagctgttcaaggtggtggatgtgatcggcgcccactaccctggcacacactctgccaaggacgccaagctgaccggcaagaagctgtggtccagcgaggacttcagcaccctgaacagcgacatgggagccggctgctggggcagaatcctgaaccagaattacatcaacggctacatgaccagcacaatcgcctggaacctggtggccagctactacgagcagctgccctacggcagatgcggcctgatgacagcccaggaaccttggagcggccactacgtggtggaaagccctgtgtgggtgtccgcccacaccacccagtttacacagcccggctggtactacctgaaaaccgtgggccacctggaaaagggcggcagctatgtggccctgacagacggactgggcaacctgaccatcatcatcgagacaatgtcccacaagcacagcaagtgcatcagaccctttctgccctacttcaacgtgtcccagcagttcgccaccttcgtgctgaagggcagcttcagcgagatccccgaactgcaagtgtggtacaccaagctgggaaagaccagcgagcggttcctgtttaagcagctggacagcctgtggctgctggacagcgacggcagctttaccctgagcctgcacgaggacgagctgtttacactgaccaccctgaccacaggccggaagggctcttaccccctgcctcctaagagccagcccttcccaagcacctacaaggacgacttcaatgtggactacccattctttagcgaggcccccaatttcgccgaccagaccggcgtgttcgagtacttcaccaacatcgaggaccccggcgagcaccacttcaccctgagacaggtgctgaaccagcggcccatcacctgggctgccgatgccagcaacaccatcagcatcatcggcgactacaactggaccaatctgacaatcaagtgcgacgtgtacatcgaaacccccgacaccggcggagtgtttatcgccggcagagtgaacaagggcgggatcctgatcagaagcgccagaggcatcttcttttggatcttcgccaacggcagctacagagtgaccggcgatctggccggctggatcatctacgctctgggcagggtggaagtgaccgccaagaaatggtacaccctgacactgactatcaagggccacttcgcctccggcatgctgaacgacaagagcctgtggaccgacatccccgtgaacttccccaagaatggctgggccgccatcggcacccacagctttgagttcgcccagttcgacaacttcctggtggaagccaccagatgatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 2208 bp, Artificial sequence
  AAV1 VP1
  CDS (1)..(2208)
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 21 - protein, 736 aa, Artificial sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 22 - DNA, 2172 bp, Artificial sequence
  AAV5 capsid VP1
  CDS (1)..(2172)
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctt

SEQ ID NO: 23 - protein, 724 aa, Artificial sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 24 - protein, 736 aa, Artificial sequence
  AAV3B VP1 Capsid
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 25 - DNA, 1227 bp, Artificial sequence
  UbC promoter
ggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggta

SEQ ID NO: 26 - DNA, 232 bp, Artificial sequence
  SV40 late polyA
cgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcg

SEQ ID NO: 27 - DNA, 1184 bp, Artificial sequence
  EF-1a promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

(19) United States
(12) Patent Application Publication
(10) Pub. No.: US 2022/0118108 A1
(43) Pub. Date: Apr. 21, 2022

(54) COMPOSITIONS USEFUL IN TREATMENT OF KRABBE DISEASE

(71) Applicant: The Trustees of the University of Pennsylvania, Philadelphia, PA (US)

(72) Inventors: James M. Wilson, Philadelphia, PA (US); Juliette Hordeaux, Philadelphia, PA (US); Nathan Katz, Stamford, CT (US)

(21) Appl. No.: 17/433,195

(22) PCT Filed: Feb. 26, 2020
(86) PCT No.: PCT/US20/19794

§ 371 Date: Aug. 23, 2021

Related U.S. Application Data
(60) Provisional application No. 62/810,708, filed on Feb. 26, 2019.
(60) Provisional application No. 62/817,482, filed on Mar. 12, 2019.
(60) Provisional application No. 62/877,707, filed on Jul. 23, 2019.
(60) Provisional application No. 62/916,652, filed on Oct. 17, 2019.

(51) Int. Cl. A61K 48/00; C12N 15/86; A61P 25/02; C12N 9/38

(52) U.S. Cl. CPC A61K 48/005; C12N 15/86; C12Y 302/01046; C12N 9/2471; A61P 25/02

(57) ABSTRACT

A composition formulated for intrathecal delivery of a recombinant adeno-associated virus (rAAV) vector comprising an AAVhu68 capsid carrying a human galactosylceramidase (GALC) gene for administration to Krabbe patients is provided. Also provided are novel gene sequences and uses thereof.

### BACKGROUND OF THE INVENTION

Adeno-associated virus (AAV), a member of the Parvovirus family, is a small non-enveloped, icosahedral virus with single-stranded linear DNA (ssDNA) genomes of about 4.7 kilobases (kb) long. The wild-type genome comprises inverted terminal repeats (ITRs) at both ends of the DNA strand, and two open reading frames (ORFs): rep and cap. Rep is composed of four overlapping genes encoding rep proteins required for the AAV life cycle, and cap contains overlapping nucleotide sequences of capsid proteins: VP1, VP2 and VP3, which self-assemble to form a capsid of an icosahedral symmetry.

Recombinant adeno-associated virus (rAAV) vectors derived from the replication defective human parvovirus have been described as suitable vehicles for gene delivery. Typically, functional rep genes and the cap gene are removed from the vector, resulting in a replication-incompetent vector. These functions are provided during the vector production system but absent in the final vector.

To date, there have been several different well-characterized AAVs isolated from human or non-human primates (NHP). It has been found that AAVs of different serotypes exhibit different transfection efficiencies, and exhibit tropism for different cells or tissues. Many different AAV clades have been described in WO 2005/033321, including clade F which is identified therein as having just three members, AAV9, AAVhu31 and AAVhu32. A structural analysis of AAV9 is provided in M. A. DiMattia et al, J. Virol. (June 2012) vol. 86 no. 12 6947-6958. This paper reports that AAV9 has 60 copies (in total) of the three variable proteins (vps) that are encoded by the cap gene and have overlapping sequences. These include VP1 (87 kDa), VP2 (73 kDa), and VP3 (62 kDa), which are present in a predicted ratio of 1:1:10, respectively. The entire sequence of VP3 is within VP2, and all of VP2 is within VP1. VP1 has a unique N-terminal domain. The refined coordinates and structure factors are available under accession no. 3UX1 from the RCSB PDB database.

Several different AAV9 variants have been engineered in order to detarget or target different tissue. See, e.g., N. Pulicheria, “Engineering Liver-detargeted AAV9 Vectors for Cardiac and Musculoskeletal Gene Transfer”, Molecular Therapy, Vol, 19, no. 6, p. 1070-1078 (June 2011). The development of AAV9 variants to deliver gene across the blood-brain barrier has also been reported. See, e.g., B. E. Deverman et al, Nature Biotech, Vol. 34, No. 2, p 204-211 (published online 1 Feb. 2016) and Caltech press release, A. Wetherston, www.neurology-central.com/2016/02/10/successful-delivery-of-genes-through-the-blood-brain-barrier/, accessed Oct. 5, 2016. See, also, WO 2016/0492301 and U.S. Pat. No. 8,734,809.

Recently, AAVhu68, which was identified following amplification of the capsid gene from a natural source, was identified as a new AAV capsid. See, e.g., WO 2018/160582. This AAV is within Clade F, as is AAV9.

Krabbe disease (globoid cell leukodystrophy; GLD) is an autosomal recessive lysosomal storage disease (LSD) caused by mutations in the gene encoding the hydrolytic enzyme galactosylceramidase (GALC) (Wenger D. A., et al. (2000) Mol Genet Metab. 70(1):1-9). This enzyme is responsible for the degradation of certain galactolipids, including galactosylceramide (ceramide) and galactosylsphingosine (psychosine), which are found almost exclusively in the myelin sheath. In Krabbe disease, GALC deficiency causes toxic accumulation of psychosine (but not galactosylceramide) in the lysosomes (Svennerholm et al., 1980). The accumulation of psychosine is particularly toxic to myelin-producing oligodendrocytes in the CNS and Schwann cells in the PNS, resulting in rapid and widespread death of these cell types. Myelin breakdown in both the CNS and PNS is accompanied by reactive astroytic gliosis and the infiltration of giant multinucleated macrophages (“globoid cells”) (Suzuki K. (2003) J Child Neurol. 18(9):595-603). Galactosylceramide does not accumulate in the absence of GALC activity due primarily to hydrolysis by another enzyme, GM1 ganglioside β-galactosidase (Kobayashi T., et al. (1985) J Biol Chem. 260(28):14982-7) and the death of oligodendrocytes contributing to an arrest in the galactosylceramide synthesis (Svennerholm L., et al. (1980) J Lipid Res. 21(1):53-64).

The only disease-modifying treatment currently available for Krabbe disease is hematopoietic stem cell transplant (HSCT), which is often provided by umbilical cord blood transplant (UCBT), allogeneic peripheral blood stem cells, or allogeneic bone marrow. There has been only modest success using HSCT to treat patients with infantile Krabbe disease, who typically present with symptoms before their first birthday. When performed after the onset of overt symptoms in infantile Krabbe disease, HSCT provides only minimal neurologic improvement and does not substantially improve survival (Escolar M. L., et al. (2005) N Engl J Med. 352(20):2069-81). HSCT can be efficacious when performed in pre-symptomatic patients, but even then, motor outcomes are poor (Escolar M. L., et al. (2005) N Engl J Med. 352(20):2069-81; Wright M. D., et al. (2017) Neurology. 89(13):1365-1372; van den Broek B. T. A., et al. (2018) Blood Adv. 2(1):49-60). Infants transplanted before 30 days of age had better survival and functional outcomes compared with those transplanted later (Allewelt H., et al. (2018) Biol Blood Marrow Transplant. 24(11):2233-2238). Presymptomatic transplantation is reported to result in significantly better outcomes with progressive central myelination, normal receptive language, attenuation of symptom severity, and longer survival compared with infantile Krabbe disease patients who were either untreated or treated after symptom onset (Escolar M. L., et al. (2005) N Engl J Med. 352(20):2069-81; Duffner P. K., et al. (2009) Genet Med. 11(6):450-4; Wright M. D., et al. (2017) Neurology. 89(13):1365-1372). Even so, most children treated before the emergence of symptoms remain well below average for height and weight, and have progressive gross motor delays ranging from mild spasticity to inability to walk independently (Escolar M. L., et al. (2005) N Engl J Med. 352(20):2069-81; Duffner P. K., et al. (2009) Genet Med. 11(6):450-4). Some children also have residual impairments, including acquired microcephaly, the need for gastrostomy, and dysarthria (Duffner P. K., et al. (2009) Genet Med. 11(6):450-4). Moreover, HSCT only appears to influence the CNS-specific disease pathology. Clinical features associated with the PNS pathology, such as peripheral neuropathy, remain unaffected by HSCT. These results highlight the limitations of HSCT, especially in early onset forms where rapid disease progression outpaces the time needed for hematopoietic stem cells to engraft, migrate to the CNS, differentiate, and provide therapeutic effect through GALC secretion and cross-correction (i.e., the process by which enzyme secreted by corrective cells is taken up by GALC-deficient cells).

There remains a need in the art for improved treatments for Krabbe disease patients.

### SUMMARY OF THE INVENTION

A composition comprising a recombinant adeno-associated virus (rAAV) is provided which comprises an AAV capsid which targets cells in the central nervous system and a vector genome comprising (i) a galactosylceramidase coding sequence encoding a mature galactosylceramidase protein having the amino acid sequence in SEQ ID NO: 10 under the control of regulatory sequences which direct expression of the protein, and (ii) AAV inverted terminal repeats necessary for packaging the vector genome in an AAV capsid, wherein the vector genome is packaged in the AAV capsid. In certain embodiments, the AAV capsid is an AAVhu68 capsid. In certain embodiments, the coding sequence has the nucleic acid sequence of SEQ ID NO: 9 or a sequence 95% to 99.9% identical thereto. In certain embodiments, the coding sequence encodes the mature protein of SEQ ID NO: 10 and an exogenous signal peptide suitable for human cells in the central nervous system. In certain embodiments, the regulatory sequences comprise: a beta-actin promoter, an intron, and/or a rabbit globin polyA. In certain embodiments, the composition comprises an rAAV having the vector genome CB7.CI.hGALC.rBG.

In certain embodiments, a recombinant adeno-associated virus is provided which comprises an AAV capsid which targets cells in the central nervous system and a vector genome comprising (i) a galactosylceramidase coding sequence encoding a mature galactosylceramidase protein having the amino acid sequence in SEQ ID NO: 10 under the control of regulatory sequences which direct expression of the mature galactosylceramidase protein, and (ii) AAV inverted terminal repeats necessary for packaging the vector genome in an AAV capsid. In certain embodiments, the AAV capsid is an AAVhu68 capsid. In certain embodiments, the coding sequence has the nucleic acid sequence of SEQ ID NO: 9 or a sequence 95% to 99.9% identical thereto. In certain embodiments, the coding sequence encodes the mature protein of SEQ ID NO: 10 and an exogenous signal peptide suitable for human cells in the central nervous system. In certain embodiments, the regulatory sequences comprise a beta-actin promoter, an intron, and/or a rabbit globin polyA. In certain embodiments, the vector genome is CB7.CI.hGALC.RBG.

In certain embodiments, a composition is provided which comprises a stock of rAAV which is useful for treatment of Krabbe disease. In certain embodiments, use of a composition comprising a stock of rAAV in preparing a medicament is provided. In certain embodiments, the composition provided is useful for treating dysfunction of peripheral nerves and/or for treating Krabbe disease. In certain embodiments, the rAAV is administrable as a co-therapy with hematopoietic stem cell therapy, bone marrow transplant, and/or substrate reduction therapy

In certain embodiments, a plasmid comprising a galactosylceramidase coding sequence encoding a signal peptide and a mature human galactosylceramidase protein having the amino acid sequence in SEQ ID NO: 10 (aa 43 to 685) is provided. In certain embodiments, the plasmid comprises a nucleic acid sequence of SEQ ID NO: 9 or a sequence 95% to 99.9% identical thereto.

In certain embodiments, use of a composition is provided for treating Krabbe disease, correcting dysfunction of peripheral nerves which causes respiratory failure and motor function loss caused by Krabbe disease, or delaying the onset or frequency of seizures caused by Krabbe disease comprising administering to a patient a composition comprising a stock of recombinant adeno-associated virus (rAAV), said rAAV comprising: (a) an AAV capsid which targets cells in the central nervous system; and (b) a vector genome comprising a galactosylceramidase coding sequence encoding a mature galactosylceramidase protein having the amino acid sequence in SEQ ID NO: 10 under the control of regulatory sequences which direct expression of the protein, said vector genome further comprising AAV inverted terminal repeats necessary for packaging the vector genome in an AAV capsid, wherein the vector genome is packaged in the AAV capsid. In certain embodiments, the patient has Late infantile Krabbe disease (LIKD). In certain embodiments, the patient has Juvenile Krabbe disease (JKD). In certain embodiments, the patient has adolescent/adult onset Krabbe disease. In certain embodiments, the rAAV is administered as a co-therapy to hematopoietic stem cell transplant (HSCT), bone marrow transplant, and/or substrate reduction therapy. In certain embodiments, the rAAV is delivered via intrathecal, intracerebroventricular, or intraparenchymal administration.

In certain embodiments, the composition provided is formulated for intrathecal, intracerebroventricular, intraparenchymal administration. In certain embodiments, the composition is administered as a single dose via a computed tomography- (CT-) guided sub-occipital injection into the cisterna magna (intra-cisterna magna).

These and other aspects of the invention will be apparent from the following detailed description of the invention.

### BRIEF DESCRIPTION OF THE DRAWINGS

FIG. 1 provides an alignment of AAV9 (SEQ ID NO: 4) and AAVhu68 (SEQ ID NO: 2) capsid sequences. The two amino acids that differ between the AAV9 and AAVhu68 capsids are located in the VP1 (67, 157) and VP2 (157) regions of the capsid. Abbreviations: AAV9, adeno-associated virus serotype 9; AAVhu68; adeno-associated virus serotype hu68; VP1, viral protein 1; VP2, viral protein.

FIG. 2 shows a schematic of the CB7.CI.hGALC.rBG vector genome. The linear map depicts the vector genome, which is designed to express human GALC under the control of the ubiquitous CB7 promoter. CB7 is composed of hybrid between a CMV IE enhancer and a chicken β-actin (CB) promoter. Abbreviations: CMV IE, cytomegalovirus immediate-early; GALC, galctosylceramidase; ITR, inverted terminal repeats; PolyA, polyadenylation; rBG, rabbit β-globin.

FIG. 3 shows a vector map for pENN.AAV.CB7.CI.RBG (p1044) with an engineered cGALC gene (cGALCco) inserted.

FIG. 4A shows the progression of neuropathological and behavioral phenotypes for the Twitcher mouse (twi/twi). Mice display an accumulation of cytotoxic psychosine followed by the infiltration of the PNS and CNS white matter by phagocytic globoid cells. Following an initial period of myelination, demyelination is observed in the PNS followed by the CNS to a lesser extent due to the death of myelin-forming Schwann cells and oligodendrocytes, respectively. Behavioral phenotypes manifest around PND 20 consisting of tremors, twitching, hind limb weakness, followed by subsequent paralysis and weight loss necessitating euthanasia around PND 40. Adapted from (Nicaise A. M., et al. (2016) J Neurosci Res. 94(11):1049-61). Abbreviations: CNS, central nervous system; PND, postnatal day; PNS, peripheral nervous system; twi, twitcher loss-of-function allele.

FIG. 4B shows a study design for evaluation of AAV.CB7.cGALCco.rBG gene therapy using the Twitcher mouse model.

FIG. 5 shows survival curves following intravenous or intracerebroventricular administration of rAAVhu68.hGALC to presymptomatic Twitcher mice. High-dose IV administration of rAAVhu68.hGALC (1.00×10¹¹ GC [equivalent to 1.00×10¹⁴ GC/kg]) to newborn Twitcher mice on PND 0 led to a median survival of 49 days (N=6). A 5-fold lower dose of rAAVhu68.hGALC (2.00×10¹⁰ GC) administered ICV to newborn Twitcher mice on PND 0 led to a median survival of 61.5 days (N=10). Survival of Twitcher mice administered rAAVhu68.hGALC was compared to that of age-matched Twitcher mice ICV-administered vehicle (PBS) as a control (N=8) Abbreviations: GC, genome copies; ICV, intracerebroventricular; IV, intravenous; N, number of animals; PND, postnatal day.

FIG. 6 shows survival curves following intracerebroventricular delivery of GALC to presymptomatic Twitcher mice using different AAV Capsids. ICV administration of the AAVhu68 vector (rAAVhu68.hGALC) at a dose of 2.00×10¹⁰ GC on PND 0 conferred a median survival of 61.5 days (N=10). ICV administration of AAV1, AAV3b, or AAV5 vectors at a dose of 2.00×10¹⁰ GC on PND 0 led to a median survival of less than 60 days (AAV1: 57 days [N=6], AAV3b: 51 days [N=9], AAV5: 51 days [N=6]), while control Twitcher mice ICV-administered vehicle only (PBS) on PND 0 displayed a median survival of 43 days (N=8). Abbreviations: AAV3b, AAV serotype 3b; AAV5, AAV serotype 5; AAV1, AAV serotype 1; and AAVhu68, AAV serotype hu68 (rAAVhu68.hGALC); GC, genome copies; ICV, intracerebroventricular; N, number of animals; PBS, phosphate-buffered saline; PND, postnatal day.

FIG. 7 shows neuromotor function following intracerebroventricular administration of different doses of rAAVhu68.hGALC to presymptomatic Twitcher mice. Presymptomatic Twitcher mice (twi/twi) were ICV-administered rAAVhu68.hGALC at a dose of 2.00×10¹⁰ GC (N=10), 5.00×10¹⁰ GC (N=12), or 1.00×10¹¹ GC (N=12) on PND 0. The doses per gram of brain mass (0.15 g in a newborn mouse) were equivalent to 1.30×10¹¹ GC/g, 3.30×10¹¹ GC/g, and 6.70×10¹¹ GC/g, respectively. As controls, PBS was ICV-administered to age-matched presymptomatic Twitcher mice (twi/twi) (N=8) and age-matched unaffected mice (twi/+ or +/+; N=14) on PND 0. On PND 35, neuromotor function was assessed by the time to fall (seconds) for mice running on an accelerating rod initially spinning at 5 RPM and increasing to 40 RPM over 120 seconds. **p<0.01 determined by a one-way ANOVA followed by a Dunn's multiple comparison test. Abbreviations: ANOVA, analysis of variance; GC, genome copies; ICV, intracerebroventricular; N, number of animals; PBS, phosphate-buffered saline; PND, postnatal day; RPM, revolutions per minute.

FIG. 8 shows survival curves following intracerebroventricular administration of different doses of rAAVhu68.hGALC to presymptomatic Twitcher mice. Control Twitcher mice (twi/twi) ICV-administered vehicle (PBS) displayed a median survival of 43 days (N=8). Twitcher mice (twi/twi) ICV-administered rAAVhu68.hGALC on PND 0 displayed a median survival of 61.5 days at a dose of 2.00×10¹⁰ GC (N=10), 99 days at a dose of 5.00×10¹⁰ GC (N=12), and 130 days at a dose of 1.00×10¹¹ GC (N=12). The rAAVhu68.hGALC doses per gram of brain mass (0.15 g in a newborn mouse) were equivalent to 1.30×10¹¹ GC/g, 3.30×10¹¹ GC/g, and 6.70×10¹¹ GC/g, respectively. Abbreviations: GC, genome copies; ICV, intracerebroventricular; N, number of animals; PBS, phosphate-buffered saline; PND, postnatal day.

FIG. 9 shows neuromotor function following intracerebroventricular administration of rAAVhu68.hGALC to symptomatic Twitcher mice. Pre-symptomatic newborn Twitcher mice (twi/twi) were ICV-administered rAAVhu68.hGALC at a dose of 1.00×10¹¹ GC (N=12) on PND 0. Early-symptomatic Twitcher mice (twi/twi) were ICV-administered rAAVhu68.hGALC at a dose of either 1.00×10¹¹ GC (N=12) or 2.00×10¹¹ GC (N=11) on PND 12. Later-symptomatic Twitcher mice (twi/twi) were ICV-administered rAAVhu68.hGALC at the higher dose of 2.00×10¹¹ GC (N=16) on PND 21. Age-matched unaffected mice (twi/+ and +/+; N=27) and affected Twitcher mice (twi/twi; N=15) were ICV-administered vehicle (PBS) as controls on PND 12. On PND 35, neuromotor function was assessed by the time to fall (seconds) for mice running on an accelerating rod initially spinning at 5 RPM and increasing to 40 RPM over 120 seconds. **p<0.01 and ***p<0.001 determined by one-way ANOVA followed by a Dunn's multiple comparison test. Abbreviations: ANOVA, analysis of variance; GC, genome copies; ICV, intracerebroventricular; N, number of animals; PBS, phosphate-buffered saline; PND, postnatal day; RPM, revolutions per minute.

FIG. 10 shows survival curves following intracerebroventricular administration of rAAVhu68.hGALC to symptomatic Twitcher mice. Early-symptomatic Twitcher mice (twi/twi) ICV-administered rAAVhu68.hGALC on PND 12 at a dose of either 1.00×10¹¹ GC (N=12) or 2.00×10¹¹ GC (N=11) had a median survival of 71 days or 81 days, respectively. Later-symptomatic Twitcher mice (twi/twi) ICV-administered rAAVhu68.hGALC on PND 21 at a dose of 2.00×10¹¹ GC (N=16) had a median survival of 51.5 days. By comparison, control Twitcher mice (twi/twi; historical controls; N=8 from Study 1 [PND 0] and N=4 from Study 2 [PND 12]) ICV-administered vehicle (PBS) on either PND 0 or PND 12 displayed a median survival of less than 50 days. Abbreviations: GC, genome copies; ICV, intracerebroventricular; N, number of animals; PBS, phosphate-buffered saline; PND, postnatal day.

FIG. 11A and FIG. 11B show clinical scoring and neuromotor function after intracerebroventricular administration of rAAVhu68.hGALC to symptomatic Twitcher mice. Early-symptomatic Twitcher mice (twi/twi; N=9) were ICV-administered rAAVhu68.hGALC at a dose of 2.00×10¹¹ GC on PND 12. Age-matched early-symptomatic Twitcher mice (twi/twi; N=9), unaffected Twitcher heterozygotes (twi/+; N=10), and wild-type mice (N=8) were ICV-adminstered PBS as controls on PND 12. (FIG. 11A) Beginning on PND 22, each mouse was evaluated daily until necropsy on PND 40 for clasping ability, gait, tremor, kyphosis, and fur quality using a clinical scoring assessment. A cumulative score was assigned to each animal, which was then normalized by AAV treatment for the duration of the study. Higher scores indicate poorer clinical status. (FIG. 11B) On PND 35, neuromotor function was assessed by the time to fall (seconds) for mice running on an accelerating rod initially spinning at 5 RPM and increasing to 40 RPM over 120 seconds. *p<0.05 determined by one-way ANOVA followed by a Dunn's multiple comparison test. Abbreviations: GC, genome copies; ICV, intracerebroventricular; N, number of animals; PBS, phosphate-buffered saline; PND, postnatal day; RPM, revolutions per minute.

FIG. 12 shows sciatic nerve histology following intracerebroventricular administration of rAAVhu68.hGALC to symptomatic Twitcher mice. Early-symptomatic Twitcher mice (twi/twi; N=9) were ICV-administered rAAVhu68.hGALC at a dose of 2.00×10¹¹ GC on PND 12. Age-matched early-symptomatic Twitcher mice (twi/twi; N=9) and unaffected wild-type mice (N=8) were ICV-administered PBS as controls on PND 12. Twenty-eight days later on PND 40, mice were necropsied, and samples of the sciatic nerve were obtained. Luxol blue and PAS reaction staining were performed on tissue sections to visualize myelin (dark staining) and globoid cells (light staining), respectively. Abbreviations: GC, genome copies; ICV, intracerebroventricular; N, number of animals; PBS, phosphate-buffered saline; PAS, Periodic acid-Schiff; PND, postnatal day.

FIG. 13A-FIG. 13C show GALC activity 28 days after intracerebroventricular administration of rAAVhu68.hGALC to symptomatic Twitcher mice on postnatal day 12. Early-symptomatic Twitcher mice (twi/twi; N=9) were ICV-administered rAAVhu68.hGALC at a dose of 2.00×10¹¹ GC on PND 12. Age-matched early-symptomatic Twitcher mice (twi/twi; N=9) and unaffected wild type mice (N=8) were ICV-adminstered PBS as controls on PND 12. Twenty-eight days later on PND 40, mice were necropsied, and samples of brain, liver, and serum were obtained. A fluorophore-based assay was used to quantify levels of GALC enzyme activity (relative FU). Abbreviations: FU, fluorescence units; GALC, galactosylceramidase; GC, genome copies; ICV, intracerebroventricular; N, number of animals; PBS, phosphate-buffered saline; PND, postnatal day.

FIG. 14A and FIG. 14B show interim survival curves following combination therapy of rAAVhu68.hGALC and bone marrow transplant. Twitcher mice (twi/twi) were treated with a BMT only (N=13, PND 10), rAAVhu68.hGALC only (N=12, PND 0 or N=13, PND 12; ICV; 1.00×10¹¹ GC), rAAVhu68.hGALC followed by a BMT (N=7; PND 0 and PND 10, respectively), or a BMT followed by rAAVhu68.hGALC (N=7; PND 10 and PND 12, respectively). Twitcher mice (twi/twi) administered PBS only served as historical controls (N=8, Study 1, PND 0; N=4, Study 2, PND 12). Interim survival results are shown, and the experiment is still ongoing. Abbreviations: BMT, bone marrow transplant; GC, genome copies; ICV, intracerebroventricular; N, number of animals; PBS, phosphate-buffered saline; PND, postnatal day

FIG. 15 shows the progression of the neuropathological and behavioral phenotypes for the Krabbe dog is presented (Wenger D. A., et al. (1999) J Hered. 90(1):138-42; Bradbury A., et al. (2016) Neuroradiol J. 29(6):417-424; Bradbury A. M., et al. (2016b) 94(11):1007-17; Bradbury A. M., et al. (2018) Hum Gene Ther. 29(7):785-801). Dashed lines indicate that data for earlier time points for the specified phenotype have not been described. *Asterisk refers to demyelination that is observed by histology. Abbreviations: BAER, brainstem auditory evoked response; CNS, central nervous system; MRI, magnetic resonance imaging; NCV, nerve conduction velocity; PNS, peripheral nervous system.

FIG. 16 shows the design of a phase ½ first-in-human clinical trial. Three subjects each are dosed in Cohort 1 (low dose) and Cohort 2 (high dose) followed by mandatory safety board reviews after subject #3 and #6. Following a 30-day interval after enrollment of the first subject in Cohort 1 and Cohort 2, the next two subjects in each cohort are enrolled concurrently. Subsequently, the 6 subjects in Cohort 3 (MTD) are enrolled simultaneously without staggered dosing. Abbreviations: FIH, first-in-human; LTFU, long-term follow-up; MTD, maximum tolerated dose.

FIG. 17 shows a decision tree for safety evaluations for the proposed Phase ½ trial. *Study suspension criteria include any event in which more than one subject experiences a Grade 3 or higher AE that is related to investigational product or ICM injection procedure as assessed by the Investigator. **Medical review is performed by the Medical Monitor in conjunction with the Principle Investigator. ***Cohort 3 subjects is enrolled simultaneously. Dosing at the MTD is not staggered with a 4-week safety observation period between each subject, and no safety board review is required after the first 3 subjects in Cohort 3 are enrolled. Abbreviations: AE, adverse event; ICM, intra-cisterna magna; MTD, maximum tolerated dose; SRT, safety review trigger.

FIG. 18A, FIG. 18B, and FIG. 18C show a table for the schedule of events for a Phase ½ trial.

FIG. 19 shows brain engraftment of GFP+donor cells in cerebellum of wildtype and Twitcher (Krabbe) mice 8 weeks post HSCT.

FIG. 20 shows a comparison of serum GALC activity in twitcher mice administered a rAAVhu68 having either an engineered GALC (cGALCco) or the native canine GALC (cGALnat) sequence. Improved survival was observed in twitcher mice administered the rAAVhu.cGALCco, compared to a rAAVhu68 having the native sequence.

FIG. 21 shows Linear vector map of the trans plasmid pAAV2/hu68.KanR (p0068). Abbreviations: AAV2, adeno-associated virus serotype 2; AAVhu68, adeno-associated virus serotype hu68; bp, base pairs; Cap, capsid; KanR, kanamycin resistance; Ori, origin of replication; Rep, replicase.

FIG. 22A and FIG. 22B show the adenovirus helper plasmid pAdDeltaF6(KanR). (FIG. 22A) Derivation of the helper plasmid pAdAF6 from parental plasmid pBHG10 through intermediates pAdAF1 and pAdAF5. (FIG. 22B) The ampicillin resistance gene in pAdAF6 was replaced by the kanamycin resistance gene to generate pAdAF6(Kan).

FIG. 23 shows a study design for evaluation of AAV.CB7.cGALCco.rBG gene therapy in Krabbe dogs.

FIG. 24A-FIG. 24C show survival and enzyme secretion into the CSF following ICM administration of AAVhu68.cGALC to Krabbe dogs. (FIG. 24A) Survival curves (ongoing). (FIG. 24B and FIG. 24C) GALC activity in CSF measured using a fluorescent substrate (Marker Gene Techonologies, Inc., Cat. No. M2774).

FIG. 25A-FIG. 25D show nerve conduction velocities (NCV) in tibial motor nerves (FIG. 25A), and radial sensory nerves (FIG. 25B), sciatic motor nerves (FIG. 25C), and ulnar motor nerves (FIG. 25D) in Krabbe dogs following administration of AAVhu68.cGALC. Periodical NCV recordings show slowed (FIG. 25A) or undetected (FIG. 25B) signals in Krabbe sham-treated dogs while all four rAAVhu68.cGALC treated animals have normalized velocities similar to an age-matched WT control dog.

FIG. 25E shows results from neurological examinations of sham and rAAVhu68.cGALC treated Krabbe dogs.

FIG. 26A-FIG. 26C show results from histology of brain sections from sham treated and AAVhu68.cGALC treated Krabbe dogs. (FIG. 26A) Luxol blue stain for myelin. (FIG. 26B) IBA1 immunostaining (microglial marker) and (FIG. 26C) quantification.

FIG. 27 shows body weight curves for wildtype (vehicle treated) and Krabbe dogs that received either vehicle or AAVhu68.cGALC.

FIG. 28A and FIG. 28B show CSF and sensory neuron safety monitoring in sham treated Krabbe and wildtype dogs, and Krabbe dogs administered AAVhu68.cGALC. (FIG. 28A) CSF pleocytosis. (FIG. 28B) Dorsal root ganglia histology from a AAVhu68.cGALC treated Krabbe dog.

FIG. 29A shows MRI measurements on sham-treated Krabbe and wildtype dogs, and Krabbe dogs administered AAVhu68.cGALC. FIG. 29B shows cumulative scoring results for MRI measurements in FIG. 29A.

FIG. 30 shows a manufacturing process flow diagram for vector production. Abbreviations: AEX, anion exchange; CRL, Charles River Laboratories; ddPCR, droplet digital polymerase chain reaction; DMEM, Dulbecco's modified Eagle medium; DNA, deoxyribonucleic acid; FFB, final formulation buffer; GC, genome copies; HEK293, human embryonic kidney 293 cells; ITFFB, intrathecal final formulation buffer; PEI, polyethylenimine; SDS-PAGE, sodium dodecyl sulfate polyacrylamide gel electrophoresis; TFF, tangential flow filtration; USP, United States Pharmacopeia; WCB, working cell bank.

FIG. 31 shows a manufacturing process flow diagram for vector formulation. Abbreviations: Ad5, adenovirus serotype 5; AUC, analytical ultracentrifugation; BDS, bulk drug substance; BSA, bovine serum albumin; CZ, Crystal Zenith; ddPCR, droplet digital polymerase chain reaction; E1A, early region 1A (gene); ELISA, enzyme-linked immunosorbent assay; FDP, final drug product; GC, genome copies; HEK293, human embryonic kidney 293 cells; ITFFB, intrathecal final formulation buffer; KanR, kanamycin resistance (gene); MS, mass spectrometry; NGS, next-generation sequencing; qPCR, quantitative polymerase chain reaction; SDS-PAGE, sodium dodecyl sulfate polyacrylamide gel electrophoresis; TCID50 50% tissue culture infective dose; UPLC, ultra-performance liquid chromatography; USP, United States Pharmacopeia.

### DETAILED DESCRIPTION OF THE INVENTION

A recombinant adeno-associated virus (rAAV) which expresses a human galactosylceramidase (GALC) protein is provided, as are compositions containing the rAAV and uses thereof. In certain embodiments, the rAAV.hGALC provides for the first time, a disease-modifying treatment for symptomatic infantile Krabbe patients (early infantile Krabbe disease, EIKD). In certain embodiments, the rAAV.hGALC provides a treatment for presymptomatic infantile patients. In certain embodiments, the rAAV.hGALC provides a therapy that can correct peripheral nerves which cause respiratory failure and motor function loss. In certain embodiments, the rAAV.hGALC provides additional options for treatment of later-onset patients for whom the benefit-risk ratio is not in favor of hematopoietic stem cell transplant (HSCT), which is currently the only disease-modifying treatment.

As used herein, a “rAAV.GALC” refers to a rAAV having an AAV capsid which has packaged therein a vector genome containing, at a minimum, a coding sequence for the galactosylceramidase protein (enzyme). rAAVhu68.GALC refers to a rAAV in which the AAV capsid is an AAVhu68 capsid, which is defined herein. The examples below also illustrate other AAV capsids.

The term “cGALC” refers to a coding sequence which expresses a canine GALC, which as used in the examples below for studies in dogs. Canine GALC has a 26 bp signal peptide and a total length of the protein of 669 amino acids.

The term “hGALC” refers to a coding sequence for a human GALC.

Isoform 1 of hGALC is the canonical sequence and is 685 amino acids in length. This amino acid sequence is reproduced in SEQ ID NO: 6. The mature protein is located at about amino acid 43 to about 685 and a signal peptide is located in positions 1 to 42, although there is some suggestion that the initiating Met is at position 17 rather than at position 1. Although multiple isoforms of GALC are known (isoforms 1-5), and over three dozen natural variants have been described, the present inventors have discovered that a variation having a threonine (T) to Alanine (A) mutation at position 641 is particularly desirable. This sequence is provided in SEQ ID NO: 10. This variant is the protein sequence encoded by the human galactosylceramidase (hGALC) coding sequence illustrated in the examples in the rAAV and vector genomes provided herein. Galactosylceramidase (GALC) is also known as galactocerebrosidase and these names are used interchangeably. In certain embodiments, this variant may be used in enzyme replacement therapy or co-therapies.

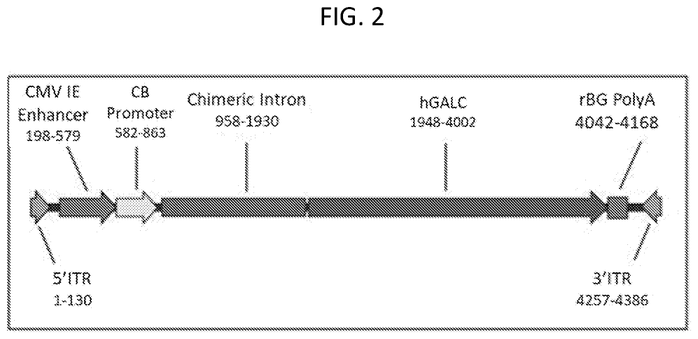

As used herein, “CB7.CI.hGALC.rBG” refers to a vector genome (e.g., as depicted in FIG. 2) that contains a coding sequence for human GALC under the control of the ubiquitous CB7 promoter and includes at least a CMV IE (cytomegalovirus immediate-early) enhancer, a chimeric intron, and a rabbit β-globin (rBG) polyA sequence, all of which are flanked by a 5′ITR and a 3′ITR. In certain embodiments, the CB7.CI.hGALC.rBG includes a GALC coding sequence encoding a mature GALC protein having the amino acid sequence of SEQ ID NO: 10. In certain embodiments, the CB7.CI.hGALC.rBG includes a coding sequence for GALC that contains the nucleic acid sequence of SEQ ID NO: 9 or a sequence 95% to 99.9% identical thereto. In yet another embodiment, the CB7.CI.hGALC.rBG vector genome includes SEQ ID NO: 19. In certain embodiments, the CB7.CI.hGALC.rBG contains a coding sequence for the mature protein of SEQ ID NO: 10 and an exogenous signal peptide.

In certain embodiments, a fusion protein is contemplated which contains at least the mature GALC with all or a portion of the native signal peptide removed (aa 1-17, or aa 1-42) and substituted with an exogenous signal peptide. Such a fusion protein may contain an exogenous signal peptide and at least the mature human GALC protein (e.g., amino acid 43 to 695 of SEQ ID NO: 6 or SEQ ID NO: 10). In certain embodiments, the fusion protein contains an exogenous signal peptide suitable for human cells in the CNS, i.e. a signal peptide that is substituted for a native signal peptide to improve production, intracellular transport, and/or secretion of the protein (i.e. hGALC) in cells present in the human CNS. Exogenous signal peptides suitable for human cells in the CNS, include, but are not limited to those natively found in an immunoglobulin (e.g., IgG), a cytokine (e.g., IL-2, IL12, IL18, or the like), insulin, albumin, β-glucuronidase, alkaline protease, von Willebrand factor (VWF), or the fibronectin secretory signal peptides (See, also, e.g., www.signalpeptide.de/index.php?m=listspdb_mammalia).

Also encompassed by the present invention are nucleic acid sequences which encode the GALC protein(s) provided herein (e.g., SEQ ID NO: 6, SEQ ID NO: 10, or fusion proteins comprising the mature GALC). In certain embodiments, a coding sequence is a cDNA sequence encoding the protein. However, also encompassed are the corresponding RNA sequences.

In certain embodiments, a nucleic acid coding sequence has the cDNA sequence of SEQ ID NO: 5 or a sequence 95% to 99.9% identical thereto, or a fragment thereof. Suitable fragments include the coding sequence for the mature protein (about nt 127 to about nt 2058), or the coding sequence for the mature protein with a fragment of the signal peptide (e.g., about nt 54 to about nt 2058). In certain embodiments, the coding sequence has the nucleic acid sequence encoding the mature hGALC of SEQ ID NO: 5 (nt 127 to 2058) or a fusion protein comprising the same and an exogenous leader, or a sequence 95% to 99.9% identical thereto. In certain embodiments, the coding sequence has the nucleic acid sequence encoding the mature hGALC of SEQ ID NO: 5 (nt 127 to 2058) or a sequence 95% to 99.9% identical thereto, or a fragment thereof comprising a fragment of the leader sequence and the mature hGALC. In certain embodiments, the coding sequence encodes a full-length human GALC protein having the amino acid sequence of SEQ ID NO: 10. In certain embodiments, the coding sequence encodes the hGALC leader (nucleic acids 1 to 126) and mature protein (encoded by nucleic acids 127 to 2058) of SEQ ID NO: 5.

In certain embodiments, the expression cassette comprises one or more miRNA target sequences that repress expression of hGALC in dorsal root ganglion (drg) (see, e.g., International Patent Application No. PCT/US19/67872, filed Feb. 12, 2020, which is incorporated herein by reference).

As used herein, Krabbe disease, also known as globoid cell leukodystrophy (GLD) is a lysosomal storage disease caused by mutation affecting the activity of galactosylceramidase (GALC), an enzyme responsible for the degradation of myelin galactolipids. Several types of Krabbe disease have been described which depend on the severity of the enzymatic deficit. From the most severe to least severe enzymatic deficit are: early infantile Krabbe disease (EIKD) defined by onset <6 months of age; late infantile Krabbe disease (LIKD) defined by onset from 7 to 12 months; juvenile Krabbe disease (JKD) defined by onset from 13 months to 10 years; and adolescent/adult onset Krabbe disease.

In certain embodiments, an effective amount of a rAAV.GALC vector increases GALC enzyme levels in the CSF to within about 30% to about 100% of normal levels. In other embodiments, an effective amount of a rAAV.GALC vector increases GALC enzyme levels in the plasma to within about 30% to about 100% of normal levels. In certain embodiments, lower amounts of increased CSF or plasma levels of GALC are observed, but an improvement is observed in one or more of the symptoms associated with Krabbe disease, as described herein.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein.

### AAVhu68

As described in the examples below, the rAAV provided herein comprises an AAVhu68 capsid. See, e.g., WO 2018/160582, which is incorporated herein by reference. AAVhu68 is within clade F. AAVhu68 (SEQ ID NO: 2) varies from another Clade F virus AAV9 (SEQ ID NO: 4) by two encoded amino acids at positions 67 and 157 of vp1. In contrast, the other Clade F AAV (AAV9, hu31, hu31) has an Ala at position 67 and an Ala at position 157.

A rAAVhu68 is composed of an AAVhu68 capsid and a vector genome. In one embodiment, a composition comprising rAAVhu68 comprises an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. SEQ ID NO: 2 provides the encoded amino acid sequence of the AAVhu68 vp1 protein. The AAVhu68 capsid contains subpopulations within the vp1 proteins, within the vp2 proteins and within the vp3 proteins which have modifications from the predicted amino acid residues in SEQ ID NO: 2. These subpopulations include, at a minimum, certain deamidated asparagine (N or Asn) residues. For example, certain subpopulations comprise at least one, two, three or four highly deamidated asparagines (N) positions in asparagine—glycine pairs in SEQ ID NO: 2 and optionally further comprising other deamidated amino acids, wherein the deamidation results in an amino acid change and other optional modifications. The various combinations of these and other modifications are described herein.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine—glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated at a referenced amino acid position, as compared to the predicted amino acid sequence at the reference amino acid position (e.g., at least 80% of the asparagines at amino acid 57 of SEQ ID NO: 2 may be deamidated based on the total vp1 proteins or 20% of the asparagines at amino acid 409 of SEQ ID NO: 2 may be deamidated based on the total vp1, vp2 and vp3 proteins). Such percentages may be determined using 2D-gel, mass spectrometry techniques, or other suitable techniques.

As provided herein, each deamidated N of SEQ ID NO: 2 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio. In certain embodiments, one or more glutamine (Q) in SEQ ID NO: 2 deamidates to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio.

Thus, an rAAVhu68 includes subpopulations within the rAAVhu68 capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAVhu68 capsid contains subpopulations of vp1, vp2 and vp3 having at least 4 to at least about 25 deamidated amino acid residue positions, of which at least 1 to 10% are deamidated as compared to the encoded amino acid sequence of SEQ ID NO: 2. The majority of these may be N residues. However, Q residues may also be deamidated.

In certain embodiments, an AAVhu68 capsid is further characterized by one or more of the following. AAVhu68 capsid proteins that comprise: AAVhu68 vp1 proteins produced by expression from a nucleic acid sequence which encodes the predicted amino acid sequence of 1 to 736 of SEQ ID NO: 2, vp1 proteins produced from SEQ ID NO: 1, or vpl proteins produced from a nucleic acid sequence at least 70% identical to SEQ ID NO:1 which encodes the predicted amino acid sequence of 1 to 736 of SEQ ID NO: 2; AAVhu68 vp2 proteins produced by expression from a nucleic acid sequence which encodes the predicted amino acid sequence of at least about amino acids 138 to 736 of SEQ ID NO:2, vp2 proteins produced from a sequence comprising at least nucleotides 412 to 2211 of SEQ ID NO:1, or vp2 proteins produced from a nucleic acid sequence at least 70% identical to at least nucleotides 412 to 2211 of SEQ ID NO: 1 which encodes the predicted amino acid sequence of at least about amino acids 138 to 736 of SEQ ID NO: 2, and/or AAVhu68 vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2211 of SEQ ID NO: 1, or vp3 proteins produced from a nucleic acid sequence at least 70% identical to at least nucleotides 607 to 2211 of SEQ ID NO: 1 which encodes the predicted amino acid sequence of at least about amino acids 203 to 736 of SEQ ID NO: 2.

Additionally or alternatively, an AAV capsid is provided which comprises a heterogeneous population of vp1 proteins optionally comprising a valine at position 157, a heterogeneous population of vp2 proteins optionally comprising a valine at position 157, and a heterogeneous population of vp3 proteins, wherein at least a subpopulation of the vp1 and vp2 proteins comprise a valine at position 157 and optionally further comprising a glutamic acid at position 67 based on the numbering of the vp1 capsid of SEQ ID NO:2. Additionally or alternatively, an AAVhu68 capsid is provided which comprises a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2, wherein: the vp1, vp2 and vp3 proteins contain subpopulations with amino acid modifications

The AAVhu68 vp1, vp2 and vp3 proteins are typically expressed as alternative splice variants encoded by the same nucleic acid sequence which encodes the full-length vp1 amino acid sequence of SEQ ID NO: 2 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes aa 203 to 736 of SEQ ID NO: 2. Additionally, or alternatively, the vp1-encoding and/or the vp2-encoding sequence may be co-expressed with the nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA or tRNA (nt 412 to 22121 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes about aa 138 to 736 of SEQ ID NO: 2.

As described herein, a rAAVhu68 has a rAAVhu68 capsid produced in a production system expressing capsids from an AAVhu68 nucleic acid which encodes the vp1 amino acid sequence of SEQ ID NO: 2, and optionally additional nucleic acid sequences, e.g., encoding a vp3 protein free of the vp1 and/or vp2-unique regions. The rAAVhu68 resulting from production using a single nucleic acid sequence vp1 produces the heterogeneous populations of vp1 proteins, vp2 proteins and vp3 proteins. More particularly, the rAAVhu68 capsid contains subpopulations within the vp1 proteins, within the vp2 proteins and within the vp3 proteins which have modifications from the predicted amino acid residues in SEQ ID NO:2. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 1, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 1). In certain embodiments, also provided is a nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA or tRNA (nt 412 to 2211 of SEQ ID NO: 1).

However, other nucleic acid sequences which encode the amino acid sequence of SEQ ID NO: 2 may be selected for use in producing rAAVhu68 capsids. In certain embodiments, the nucleic acid sequence has the nucleic acid sequence of SEQ ID NO: 1 or a sequence at least 70% to 99% identical, at least 75%, at least 80%, at least 85%, at least 90%, at least 95%, at least 97%, or at least 99% identical to SEQ ID NO: 1 which encodes SEQ ID NO: 2. In certain embodiments, the nucleic acid sequence has the nucleic acid sequence of SEQ ID NO: 1 or a sequence at least 70% to 99%, at least 75%, at least 80%, at least 85%, at least 90%, at least 95%, at least 97%, or at least 99% identical to about nt 412 to about nt 2211 of SEQ ID NO: 1 which encodes the vp2 capsid protein (about aa 138 to 736) of SEQ ID NO: 2. In certain embodiments, the nucleic acid sequence has the nucleic acid sequence of about nt 607 to about nt 2211 of SEQ ID NO: 1 or a sequence at least 70% to 99.%, at least 75%, at least 80%, at least 85%, at least 90%, at least 95%, at least 97%, or at least 99% identical to about nt 607 to about nt 2211 SEQ ID NO: 1 which encodes the vp3 capsid protein (about aa 203 to 736) of SEQ ID NO: 2.

It is within the skill in the art to design nucleic acid sequences encoding this rAAVhu68 capsid, including DNA (genomic or cDNA), or RNA (e.g., mRNA). In certain embodiments, the nucleic acid sequence encoding the AAVhu68 vp1 capsid protein is provided in SEQ ID NO: 1. In other embodiments, a nucleic acid sequence of 70% to 99.9% identity to SEQ ID NO: 1 may be selected to express the AAVhu68 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 1. Such nucleic acid sequences may be codon-optimized for expression in a selected system (i.e., cell type) can be designed by various methods. This optimization may be performed using methods which are available on-line (e.g., GeneArt), published methods, or a company which provides codon optimizing services, e.g., DNA2.0 (Menlo Park, Calif.). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, the asparagine (N) in N-G pairs in the rAAVhu68 vp 1, vp2 and vp3 proteins are highly deamidated. In the case of the rAAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to ˜20% across various lots. The deamidation levels were initially identified using a trypsin digest and verified with a chymotrypsin digestion.

In certain embodiments, an rAAVhu68 capsid contains subpopulations of AAV vp1, vp2 and/or vp3 capsid proteins having at least four asparagine (N) positions in the rAAVhu68 capsid proteins which are highly deamidated. In certain embodiments, about 20 to 50% of the N-N pairs (exclusive of N-N-N triplets) show deamidation. In certain embodiments, the first N is deamidated. In certain embodiments, the second N is deamidated. In certain embodiments, the deamidation is between about 15% to about 25% deamidation. Deamidation at the Q at position 259 of SEQ ID NO: 2 is about 8% to about 42% of the AAVhu68 vp1, vp2 and vp3 capsid proteins of an AAVhu68 protein.

In certain embodiments, the rAAVhu68 capsid is further characterized by an amidation in D297 the vp1, vp2 and vp3 proteins. In certain embodiments, about 70% to about 75% of the D at position 297 of the vp1, vp2 and/or vp3 proteins in a AAVhu68 capsid are amidated, based on the numbering of SEQ ID NO: 2. In certain embodiments, at least one Asp in the vp1, vp2 and/or vp3 of the capsid is isomerized to D-Asp. Such isomers are generally present in an amount of less than about 1% of the Asp at one or more of residue positions 97, 107, 384, based on the numbering of SEQ ID NO: 2.

In certain embodiments, a rAAVhu68 has an AAVhu68 capsid having vp1, vp2 and vp3 proteins having subpopulations comprising combinations of one, two, three, four or more deamidated residues at the positions set forth in the table below. Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH₂ groups). The percent deamidation of a particular peptide is determined mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

| | |
|---|---|
| Deamidation Based on | |
| Predicted AAVHu68 | Average % Based on VP1/VP2/VP3 |
| (SEQ ID NO: 2) | Proteins in AAVhu68 Capsid |

| Deamidated Residue + 1 | Broad Range of | Narrow |
|---|---|---|
| (Neighboring AA) | Percentages (%) | Ranges (%) |
| N57 | 78 to 100% | 80 to 100, |
| (N-G) | | 85 to 97 |
| N66 | 0 to 5 | 0, 1 to 5 |
| (N-E) | | |
| N94 | 0 to 15, | 0, 1 to 15, |
| (N-H) | | 5 to 12, 8 |
| N113 | 0 to 2 | 0, 1 to 2 |
| (N-L) | | |
| ~N253 | 10 to 25 | 15 to 22 |
| (N-N) | | |
| Q259 | 8 to 42 | 10 to 40, |
| (Q-I) | | 20 to 35 |
| ~N270 | 12 to 30 | 15 to 28 |
| (N-D) | | |
| ~N304 | 0 to 5 | 1 to 4 |
| (N-N) (position | | |
| 303 also N) | | |
| N319 | 0 to 5 | 0, 1 to 5, |
| (N-I) | | 1 to 3 |
| N329 * | 65 to 100 | 70 to 95, |
| (N-G)*(position | | 85 to 95, |
| 328 also N) | | 80 to 100, |
| | | 85 to 100, |
| N336 | 0 to 100 | 0, 1 to 10, |
| (N-N) | | 25 to 100, |
| | | 30 to 100, |
| | | 30 to 95 |
| ~N409 | 15 to 30 | 20 to 25 |
| (N-N) | | |
| N452 | 75 to 100 | 80 to 100, |
| (N-G) | | 90 to 100, |
| | | 95 to 100, |
| N477 | 0 to 8 | 0, 1 to 5 |
| (N-Y) | | |
| N512 | 65 to 100 | 70 to 95, |
| (N-G) | | 85 to 95, |
| | | 80 to 100, |
| | | 85 to 100, |
| ~N515 | 0 to 25 | 0, 1 to 10, |
| (N-S) | | 5 to 25, |
| | | 15 to 25 |
| ~Q599 | 1 to 20 | 2 to 20, |
| (Asn-Q-Gly) | | 5 to 15 |
| N628 | 0 to 10 | 0, 1 to 10, |
| (N-F) | | 2 to 8 |
| N651 | 0 to 3 | 0, 1 to 3 |
| (N-T) | | |
| N663 | 0 to 5 | 0, 1 to 5, |
| (N-K) | | 2 to 4 |
| N709 | 0 to 25 | 0, 1 to 22, |
| (N-N) | | 15 to 25 |
| N735 | 0 to 40 | 0. 1 to 35, |
| | | 5 to 50, |
| | | 20 to 35 |

In certain embodiments, the AAVhu68 capsid is characterized by having capsid proteins in which at least 45% of N residues are deamidated at least one of positions N57, N329, N452, and/or N512 based on the numbering of amino acid sequence of SEQ ID NO: 2. In certain embodiments, at least about 60%, at least about 70%, at least about 80%, or at least 90% of the N residues at one or more of these N-G positions (i.e., N57, N329, N452, and/or N512, based on the numbering of amino acid sequence of SEQ ID NO: 2) are deamidated. In these and other embodiments, an AAVhu68 capsid is further characterized by having a population of proteins in which about 1% to about 20% of the N residues have deamidations at one or more of positions: N94, N253, N270, N304, N409, N477, and/or Q599, based on the numbering of amino acid sequence of SEQ ID NO: 2. In certain embodiments, the AAVhu68 comprises at least a subpopulation of vp 1, vp2 and/or vp3 proteins which are deamidated at one or more of positions N35, N57, N66, N94, N113, N252, N253, Q259, N270, N303, N304, N305, N319, N328, N329, N336, N409, N410, N452, N477, N515, N598, Q599, N628, N651, N663, N709, N735, based on the numbering of amino acid sequence of SEQ ID NO: 2, or combinations thereof. In certain embodiments, the capsid proteins may have one or more amidated amino acids.

Still other modifications are observed, most of which do not result in conversion of one amino acid to a different amino acid residue. Optionally, at least one Lys in the vp1, vp2 and vp3 of the capsid are acetylated. Optionally, at least one Asp in the vp1, vp2 and/or vp3 of the capsid is isomerized to D-Asp. Optionally, at least one S (Ser, Serine) in the vp1, vp2 and/or vp3 of the capsid is phosphorylated. Optionally, at least one T (Thr, Threonine) in the vp1, vp2 and/or vp3 of the capsid is phosphorylated. Optionally, at least one W (trp, tryptophan) in the vp1, vp2 and/or vp3 of the capsid is oxidized. Optionally, at least one M (Met, Methionine) in the vp1, vp2 and/or vp3 of the capsid is oxidized. In certain embodiments, the capsid proteins have one or more phosphorylations. For example, certain vpl capsid proteins may be phosphorylated at position 149.

In certain embodiments, an rAAVhu68 capsid comprises a heterogeneous population of vp1 proteins which are the product of a nucleic acid sequence encoding the amino acid sequence of SEQ ID NO: 2, wherein the vp1 proteins comprise a Glutamic acid (Glu) at position 67 and/or a valine (Val) at position 157; a heterogeneous population of vp2 proteins optionally comprising a valine (Val) at position 157; and a heterogeneous population of vp3 proteins. The AAVhu68 capsid contains at least one subpopulation in which at least 65% of asparagines (N) in asparagine—glycine pairs located at position 57 of the vp1 proteins and at least 70% of asparagines (N) in asparagine—glycine pairs at positions 329, 452 and/or 512 of the vp1, v2 and vp3 proteins are deamidated, based on the residue numbering of the amino acid sequence of SEQ ID NO: 2, wherein the deamidation results in an amino acid change.

As discussed in more detail herein, the deamidated asparagines may be deamidated to aspartic acid, isoaspartic acid, an interconverting aspartic acid/isoaspartic acid pair, or combinations thereof. In certain embodiments, the rAAVhu68 are further characterized by one or more of: (a) each of the vp2 proteins is independently the product of a nucleic acid sequence encoding at least the vp2 protein of SEQ ID NO: 2; (b) each of the vp3 proteins is independently the product of a nucleic acid sequence encoding at least the vp3 protein of SEQ ID NO: 2; (c) the nucleic acid sequence encoding the vp1 proteins is SEQ ID NO: 1, or a sequence at least 70% to at least 99% (e.g., at least 85%, at least 90%, at least 95%, at least 97%, at least 98% or at least 99%) identical to SEQ ID NO: 1 which encodes the amino acid sequence of SEQ ID NO: 2. Optionally that sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 2 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes aa 203 to 736 of SEQ ID NO: 2. Additionally, or alternatively, the vp1-encoding and/or the vp2-encoding sequence may be co-expressed with the nucleic acid sequence which encodes the AAVhu68 vp2 amino acid sequence of SEQ ID NO: 2 (about aa 138 to 736) without the vp1-unique region (about aa 1 to about 137), or a strand complementary thereto, the corresponding mRNA or tRNA (nt 412 to 2211 of SEQ ID NO: 1), or a sequence at least 70% to at least 99% (e.g., at least 85%, at least 90%, at least 95%, at least 97%, at least 98% or at least 99%) identical to SEQ ID NO: 1 which encodes about aa 138 to 736 of SEQ ID NO: 2.

Additionally or alternatively, the rAAVhu68 capsid comprises at least a subpopulation of vp 1, vp2 and/or vp3 proteins which are deamidated at one or more of positions N57, N66, N94, N113, N252, N253, Q259, N270, N303, N304, N305, N319, N328, N329, N336, N409, N410, N452, N477, N512, N515, N598, Q599, N628, N651, N663, N709, based on the numbering of SEQ ID NO:2, or combinations thereof; (e) rAAVhu68 capsid comprises a subpopulation of vp1, vp2 and/or vp3 proteins which comprise 1% to 20% deamidation at one or more of positions N66, N94, N113, N252, N253, Q259, N270, N303, N304, N305, N319, N328, N336, N409, N410, N477, N515, N598, Q599, N628, N651, N663, N709, based on the numbering of SEQ ID NO:2, or combinations thereof; (f) the rAAVhu68 capsid comprises a subpopulation of vp1 in which 65% to 100% of the N at position 57 of the vp1 proteins, based on the numbering of SEQ ID NO:2, are deamidated; (g) the rAAVhu68 capsid comprises subpopulation of vp1 proteins in which 75% to 100% of the N at position 57 of the vp1 proteins are deamidated; (h) the rAAVhu68 capsid comprises subpopulation of vp1 proteins, vp2 proteins, and/or vp3 proteins in which 80% to 100% of the N at position 329, based on the numbering of SEQ ID NO:2, are deamidated; (i) the rAAVhu68 capsid comprises subpopulation of vp1 proteins, vp2 proteins, and/or vp3 proteins in which 80% to 100% of the N at position 452, based on the numbering of SEQ ID NO:2, are deamidated; (j) the rAAVhu68 capsid comprises subpopulation of vpl proteins, vp2 proteins, and/or vp3 proteins in which 80% to 100% of the N at position 512, based on the numbering of SEQ ID NO: 2, are deamidated; (k) the rAAV comprises about 60 total capsid proteins in a ratio of about 1 vp1 to about 1 to 1.5 vp2 to 3 to 10 vp3 proteins; (1) the rAAV comprises about 60 total capsid proteins in a ratio of about 1 vp1 to about 1 vp2 to 3 to 9 vp3 proteins.

In certain embodiments, the AAVhu68 is modified to change the glycine in an asparagine-glycine pair, in order to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amide groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAVhu68 amino acid sequence. In certain embodiments, such modifications are not made in all four of the asparagine-glycine pairs. Thus, a method is provided for reducing deamidation of rAAVhu68 and/or engineered rAAVhu68 variants having lower deamidation rates. Additionally, one or more other amide amino acids may be changed to a non-amide amino acid to reduce deamidation of the rAAVhu68.

These amino acid modifications may be made by conventional genetic engineering techniques. For example, a nucleic acid sequence containing modified AAVhu68 vp codons may be generated in which one to three of the codons encoding glycine at position 58, 330, 453 and/or 513 in SEQ ID NO: 2 (asparagine-glycine pairs) are modified to encode an amino acid other than glycine. In certain embodiments, a nucleic acid sequence containing modified asparagine codons may be engineered at one to three of the asparagine-glycine pairs located at position 57, 329, 452 and/or 512 in SEQ ID NO: 2, such that the modified codon encodes an amino acid other than asparagine. Each modified codon may encode a different amino acid. Alternatively, one or more of the altered codons may encode the same amino acid. In certain embodiments, these modified AAVhu68 nucleic acid sequences may be used to generate a mutant rAAVhu68 having a capsid with lower deamidation than the native hu68 capsid. Such mutant rAAVhu68 may have reduced immunogenicity and/or increase stability on storage, particularly storage in suspension form. As used herein, a “codon” refers to three nucleotides in a sequence which encodes an amino acid.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

rAAVhu68 capsids may be useful in certain embodiments. For example, such capsids may be used in generating monoclonal antibodies and/or generating reagents useful in assays for monitoring AAVhu68 concentration levels in gene therapy patients. Techniques for generating useful anti-AAVhu68 antibodies, labelling such antibodies or empty capsids, and suitable assay formats are known to those of skill in the art.

In certain embodiments, provided herein is a nucleic acid sequence of SEQ ID NO: 1 or a sequence at least 70%, at least 75%, at least 80%, at least 85%, at least 90%, at least 95%, at least 97%, at least 99%, which encodes the vp1 amino acid sequence of SEQ ID NO: 2 with a modification (e.g., deamidated amino acid) as described herein. In certain embodiments, the vp1 amino acid sequence is reproduced in SEQ ID NO: 2.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10: 6381-6388, which identifies Clades A, B, C, D, E and F, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence of SEQ ID NO: 3 which encodes the vp1 amino acid sequence of SEQ ID NO: 4 (GenBank accession: AAS99264). These splice variants result in proteins of different length of SEQ ID NO: 4. In certain embodiments, “AAV9 capsid” includes an AAV having an amino acid sequence which is 99% identical to AAS99264 or 99% identical to SEQ ID NO: 4. See, also U.S. Pat. No. 7,906,111 and WO 2005/033321. As used herein “AAV9 variants” include those described in, e.g., WO2016/049230, U.S. Pat. No. 8,927,514, US 2015/0344911, and U.S. Pat. No. 8,734,809.

Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

rAAV Vectors

As indicated above, the AAVhu68 sequences and proteins are useful in production of rAAV, and are also useful in recombinant AAV vectors which may be antisense delivery vectors, gene therapy vectors, or vaccine vectors. Additionally, the engineered AAV capsids described herein, e.g., those having mutant amino acids at position 67, 157, or both relative to the numbering of the vp1 capsid protein in SEQ ID NO: 2, may be used to engineer rAAV vectors for delivery of a number of suitable nucleic acid molecules to target cells and tissues.

Genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest.

In particular, the present disclosure provides rAAV comprising a coding sequence of human galactosylceramidase (GALC). In some embodiments, the coding sequence is an engineered GALC coding sequence. In some embodiments, the coding sequence is the sequence of cGALC gene (cGALCco) of SEQ ID NO: 9.

The nucleic acid coding sequence is operatively linked to regulatory components in a manner which permits transgene transcription, translation, and/or expression in a cell of a target tissue. In some embodiments, the regulatory sequences comprise a beta-actin promoter, an intron, and a rabbit globin polyA. In some embodiments, the regulatory sequences comprise SEQ ID NO: 13. In some embodiments, the regulatory sequences comprise SEQ ID NO: 15. In some embodiments, the regulatory sequences comprise SEQ ID NO: 16.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, N.Y. (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. A shortened version of the 5′ ITR, termed AITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter. In addition to a promoter a vector may contain one or more other appropriate transcription initiation, termination, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., MA Zanta-Boussif, et al, Gene Therapy (2009) 16: 605-619.

These rAAVs are particularly well suited to gene delivery for therapeutic purposes and for immunization, including inducing protective immunity. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art.

rAAV Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J Gene Med. 10:717-733; and the references cited below, each of which is incorporated herein by reference in its entirety. For packaging a gene into virions, the ITRs are the only AAV components required in cis in the same construct as the nucleic acid molecule containing the expression cassette(s). The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, N.Y. (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant rAAVhu68 is provided. Such a cell culture contains a nucleic acid which expresses the rAAVhu68 capsid protein in the host cell; a nucleic acid molecule suitable for packaging into the rAAVhu68 capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAVhu68 capsid. In one embodiment, the cell culture is composed of mammalian cells (e.g., human embryonic kidney 293 cells, among others) or insect cells (e.g., baculovirus).

Suitably, the rep functions are provided by an AAV which is from the same source as the ITRs which are present in the vector genome, or from another source which packages the vector genome into the AAV capsid (e.g., AAVhu68). In certain embodiments, the rep protein is from AAV2. However, in other embodiments In another embodiment, the rep protein is a heterologous rep protein other than AAVhu68rep, for example but not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Any of these AAVhu68 or mutant AAV capsid sequences may be under the control of exogenous regulatory control sequences which direct expression thereof in a production cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In certain embodiments, the manufacturing process for rAAV.hGALC involves transient transfection of HEK293 cells with plasmid DNA. A single batch or multiple batches are produced by PEI-mediated triple transfection of HEK293 cells in PALL iCELL is bioreactors. Harvested AAV material are purified sequentially by clarification, TFF, affinity chromatography, and anion exchange chromatography in disposable, closed bioprocessing systems where possible.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016 and its priority documents, U.S. Patent Application Nos. 62/322,071, filed Apr. 13, 2016 and 62/226,357, filed Dec. 11, 2015 and entitled “Scalable Purification Method for AAV9”, which is incorporated by reference herein. Purification methods for AAV8, International Patent Application No. PCT/US2016/065976, filed Dec. 9, 2016 and its priority documents U.S. Patent Application Nos. 62/322,098, filed Apr. 13, 2016 and 62/266,341, filed Dec. 11, 2015, and rh10, International Patent Application No. PCT/US16/66013, filed Dec. 9, 2016 and its priority documents, U.S. Patent Application No. 62/322,055, filed Apr. 13, 2016 and 62/266,347, entitled “Scalable Purification Method for AAVrh10”, also filed Dec. 11, 2015, and for AAV1, International Patent Application No. PCT/US2016/065974, filed Dec. 9, 2016 and its priority documents U.S. Patent Application Nos. 62/322,083, filed Apr. 13, 2016 and 62/26,351, for “Scalable Purification Method for AAV1”, filed Dec. 11, 2015, are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Vivol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, Calif.) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods. 2014 April; 25(2):115-25. doi: 10.1089/hgtb.2013.131. Epub 2014 Feb. 14.

In brief, the method for separating rAAVhu68 particles having packaged genomic sequences from genome-deficient AAVhu68 intermediates involves subjecting a suspension comprising recombinant AAVhu68 viral particles and AAVhu689 capsid intermediates to fast performance liquid chromatography, wherein the AAVhu68 viral particles and AAVhu68 intermediates are bound to a strong anion exchange resin equilibrated at a pH of 10.2, and subjected to a salt gradient while monitoring eluate for ultraviolet absorbance at about 260 and about 280. Although less optimal for rAAV9hu68, the pH may be in the range of about 10.0 to 10.4. In this method, the AAVhu68 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/hu68 serotype. Under these ionic conditions, a significant percentage of residual cellular DNA and proteins flow through the column, while AAV particles are efficiently captured.

### Compositions and Uses

Provided herein are compositions containing at least one rAAV.hGALC stock (e.g., an rAAVhu68 stock or a mutant rAAV stock) and an optional carrier, excipient and/or preservative. An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, anocapsules, microparticles, microspheres, lipid particles, vesicles, and the like, may be used for the introduction of the compositions of the present invention into suitable host cells, in particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney,), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range. In one embodiment, for human application the dose can range from 1.4×10¹³ to about 4×10¹⁴ GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 pt. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

Therapeutically effective intrathecal/intracisternal doses of the rAAV.hGALC range from about 1×10¹¹ to 7.0×10¹⁴ GC (flat doses)—the equivalent of 10⁹ to 5×10¹⁰ GC/g brain mass of the patient. Alternatively, the following therapeutically effective flat doses can be administered to patients of the indicated age group:
Newborns: about 1×10¹¹ to about 3×10¹⁴ GC;
3-9 months: about 6×10¹² to about 3×10¹⁴ GC;
9 months-6 years: about 6×10¹² to about 3×10¹⁴ GC;
3-6 years: about 1.2×10¹² to about 6×10¹⁴ GC;
6-12 years: about 1.2×10¹² to about 6×10¹⁴ GC;
12+years: about 1.4×10¹² to about 7.0×10¹⁴GC;
18+years (adult): about 1.4×10¹² to about 7.0×10¹⁴ GC.

In certain embodiments, the dose may be in the range of about 1×10⁹ GC/g brain mass to about 1×10¹² GC/g brain mass. In certain embodiments, the dose may be in the range of about 3×10¹⁰ GC/g brain mass to about 3×10¹¹ GC/g brain mass. In certain embodiments, the dose may be in the range of about 5×10¹⁰ GC/g brain mass to about 1.85×10¹¹ GC/g brain mass. For scaling between infants and adolescent/adults, brain mass is in some instances estimated to be about 600 g to about 800 g for a four to 12 month old; about 800 g to about 1000 g for a nine month to 18 month old, about 1000 g to about 1100 g for an 18 month old to a three year old; 1100 g to about 1300 g an adolescent or adult humans, or about 1300 g for an adult human.

In one embodiment, the viral constructs may be delivered in doses of from at least about least 1×10⁹ GCs to about 1×10¹⁵, or about 1×10¹¹ to 5×10¹² GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage is adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other routes of delivery.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

In one example, the formulation may contain, e.g., buffered saline solution comprising one or more of sodium chloride, sodium bicarbonate, dextrose, magnesium sulfate (e.g., magnesium sulfate.7H₂O), potassium chloride, calcium chloride (e.g., calcium chloride.2H₂O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B® solution [Lukare Medical].

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2.

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

As used herein, the term Computed Tomography (CT) refers to radiography in which a three-dimensional image of a body structure is constructed by computer from a series of plane cross-sectional images made along an axis.

The rAAV.GALC vectors and compositions provided herein are useful for correcting conditions associated with deficient levels of GALC enzymatic activity. In certain embodiments, the rAAV.GALC vectors and compositions provided herein are useful for treating dysfunction of peripheral nerves caused by deficiencies in GALC, useful in treating respiratory failure and/or motor function loss caused by GALC deficiencies, useful in treating Krabbe disease, and/or useful in treating symptoms associated with Krabbe disease in patients.

In certain embodiments, a composition comprising an effective amount of rAAV.hGALC is administered to a patient who is less than 6 months of age who has early infantile Krabbe disease (EIKD). In certain embodiments, the patient is less than 6 months of age and has GALC enzymatic deficiencies which are less severe than EIKD.

In certain embodiments, a composition comprising an effective amount of rAAV.hGALC is administered to a patient who is older than 6 months of age, e.g., 7 months to about 12 months who has late infantile Krabbe disease (LIKD). In certain embodiments, the patient is older than 6 months, or about 7 months to 12 months of age, and has GALC enzymatic deficiencies which are less severe than LIKD.

In certain embodiments, the patient is over a year old (e.g., from 13 months to 10 years) of age and has juvenile Krabbe disease (JKD). In certain embodiments, the patient is from 13 months to 10 years of age and has GALC enzymatic deficiencies which are less severe than JKD.

In certain embodiments, the patient is over 10 years of age (e.g., from over 10 years to 12 years, or from 10 years to 18 years or older) of age and has adolescent or adult onset Krabbe disease.

In any of the embodiments described above, the rAAV.hGALC therapy provided herein may be administered as a co-therapy with hematopoietic stem cell replacement therapy, bone marrow transplant (BMT), and/or substrate reduction therapy (SRT). In certain embodiments, the rAAV.hGALC therapy (e.g., EIKD) is followed by a co-therapy such as HSCT or BMT, or enzyme replacement therapy. In certain embodiments, the therapy results in rapid enzyme production following administration of the vector, including within 1 week post-treatment.

In certain embodiments, enzyme replacement therapy involves administration of the human GALC protein of SEQ ID NO: 10. In other embodiments, other hGALC protein variants (e.g., such as the canonical sequence identified herein, or an engineered protein), may be used in enzyme replacement therapy. Combinations of different hGALC proteins may be used in enzyme replacement therapy. In such embodiments, the hGALC protein may be produced in vitro using a suitable production system See, e.g., C. Lee et al, 2005 Oct. 1, Enzyme replacement therapy results in substantial improvements in early clinical phenotype in a mouse model of globoid cell leukodystrophy, FASEB journal, The FASEB Journal 19(11):1549-51, October 2005]. The hGALC proteins may be formulated for delivery (e.g., suspended in a physiologically compatible saline solution) by any suitable route including, but not limited to intravenous, intraperitoneal, or an intrathecal route. Suitable doses may range from 1 mg/kg to 20 mg/kg, or 5 mg/kg to 10 mg/kg and may be readministered once a week, or more or less frequently, as needed (e.g., once every other day, once every two weeks, etc). Using CSF administration of the hAAVhu68.GALC vector, GALC levels in brain and serum can be supraphysiological without toxicity and improved neuromotor function and myelination in CNS and PNS may be observed. When newborn CSF administration is followed by bone marrow transplant in a postnatal conditioned animal model, survival can be extended (e.g., to >300 days) in the absence of overt signs. In a presymptomatic Krabbe patient, a single cisterna magna injection of AAV.cGALC may provide phenotypic correction, survival increase, nerve conduction normalization, and/or improved brain MRI.

In certain embodiments, the rAAV.hGALC therapy is provided following HSCT or BMT (e.g., LIKD or JKD). However, in certain embodiments, the rAAV.hGALC provides sufficient GALC levels that HSCT or BMT are not required.

The goal of treatment is to functionally replace the patient's defective GALC via rAAV-based CNS- and PNS-directed gene therapy. Efficacy of the therapy for EIKD or LIKD patients can be measured by assessing improvement in one or more of the symptoms of EIKD or LIKD: crying and irritability, spasticity, fisted hands, loss of smiling, poor head control and feeding difficulties; mental and motor deterioration, hyper or hypotonicity, seizures, blindness, deafness, and increased survival (for EIKD, without treatment, death typically occurs before the age of 2; for LIKD, survival may increase to 3-5 years of age). Additionally, for these and other Krabbe patients, efficacy of treatment may be assessed by: a decrease in dysmyelination and demyelination affecting both peripheral nerves and CNS white matter (deep cerebral white matter and dentate/cerebellar white matter) which can be monitored via imaging (e.g., magnetic resonance imaging (MRI)); a decrease in abnormal nerve conduction velocity (NCV) and/or brainstem auditory evoked potentials (BAEPs); increased levels of GALC may be observed in cerebrospinal fluid and/or plasma; and/or decreased accumulation of psychosine.

A composition comprising a recombinant adeno-associated virus (rAAV) is provided which comprises an AAV capsid which targets cells in the central nervous system and which has packaged therein a vector genome comprising a galactosylceramidase coding sequence encoding a mature galactosylceramidase protein having the amino acid sequence in SEQ ID NO: 10 under the control of regulatory sequences which direct expression of the protein, said vector genome further comprising AAV inverted terminal repeats necessary for packaging the vector genome in an AAV capsid.

In certain embodiments, a composition useful for treating Krabbe disease is provided which comprises rAAVhu68 having a vector genome of CB7.CI.hGALC.rBG. In one embodiment, the vector genome has the coding sequence of (SEQ ID NO: 19).

In certain embodiments, use of a composition is provided in a method for correcting dysfunction of peripheral nerves caused by a GALC deficiency and/or a method for treating respiratory failure and motor function loss caused by a GALC deficiency. In certain embodiments, the method comprises administering a composition comprising a stock of recombinant adeno-associated virus (rAAV) which comprises: (a) an AAV capsid which targets cells in the central nervous system and which has a vector genome of (b) packaged therein; and (b) a vector genome comprising a galactosylceramidase coding sequence encoding a mature galactosylceramidase protein having the amino acid sequence in SEQ ID NO: 10 under the control of regulatory sequences which direct expression of the protein, wherein the vector genome further comprises AAV inverted terminal repeats necessary for packaging the vector genome in an AAV capsid.

In certain embodiments, a rAAV.hGALC composition as provided herein is delivered intrathecally for treatment of a patient with early infantile Krabbe disease. In certain embodiments, a composition as provided herein is delivered intrathecally for treatment of a patient with late infantile Krabbe disease (LIKD). In certain embodiments, a rAAV.hGALC composition as provided herein is delivered intrathecally for treatment of a patient with Juvenile Krabbe disease (JKD). In certain embodiments, rAAV.hGALC composition as provided herein is delivered intrathecally for treatment of a patient with adolescent or adult onset Krabbe disease. In certain embodiments, the rAAV.hGALC composition is administered as a co-therapy to hematopoietic stem cell transplant (HSCT), bone marrow transplant, and/or substrate reduction therapy. In certain embodiments, the rAAV.hGALC composition is administered as a single dose via a computed tomography-(CT-) guided sub-occipital injection into the cisterna magna (intra-cisterna magna).

Administration of rAAV.hGALC stabilizes disease progression as measured by survival, preventing loss of developmental and motor milestone potentially supporting acquisition of new milestones, onset and frequency of seizures. Thus, in certain embodiments, methods for monitoring treatment are provided wherein endpoints are measured at, for example, 30 days, 90 days and/or 6 months, and then, for example, every 6 months during the 2-year short-term follow-up period. In certain embodiments, measurement frequency decreases to once every 12 months during the long-term extension. Given the severity of disease in the target population, subjects may have achieved motor skills by enrollment, developed and subsequently lost other motor milestones, or not yet shown signs of motor milestone development. Assessments therefore track age-at-achievement and age-at-loss for all milestones. In certain embodiments, milestones include, for example, one or more of sitting without support, hand-and-knees crawling, standing with assistance, walking with assistance, standing alone, and/or walking alone. In certain embodiments, treatment results in a delayed onset of seizure activity and/or a decrease in the frequency of seizure events.

In certain embodiments, methods of monitoring treatment in a subject uses clinical scales to quantify the effects of treatment on development and changes in adaptive behaviors, cognition, language, motor function, and/or health-related quality of life. Scales and domains include, for example, the Bayley Scales of Infant and Toddler Development (assesses development of infant and toddlers across five domains: cognitive, language, motor, social-emotional, and adaptive behavior), the Vineland Adaptive Behavior Scales (Edition III) (assesses adaptive behavior from birth through adulthood (0-90 years) across five domains: communication, daily living skills, socialization, motor skills, and maladaptive behavior), the Peabody Developmental Motor Scales-Second Edition (measures interrelated motor function from birth to children five years of age; assessments focus on six domains: reflexes, stationary, locomotion, object manipulation, grasping, and visual-motor integration), the Infant Toddler Quality of Life Questionnaire (ITQOL) (parent-reported measure of health-related quality of life designed for infants 2 months of age up to toddlers 5 years of age), and the Mullen Scales of Early Learning (assesses language, motor, and perceptual abilities in infants and toddlers up to 68 months of age). In certain embodiments, the effects of treatments are monitored or measured by evaluating changes in myelination, functional outcomes related to myelination, and potential disease biomarkers. In certain embodiments, central and peripheral demyelination slow or cease in progression following treatment of a subject. Central demyelination may be tracked by diffusion-tensor magnetic resonance imaging (DT-MRI) anisotropy measurements of white matter regions and fiber tracking of corticospinal motors tracts, changes in which are indicators of disease state and progression. Peripheral demyelination may be measured indirectly via nerve conduction velocity (NCV) studies on the motor nerves (deep peroneal, tibial, and ulnar nerves) and sensory nerves (sural, and median nerves) to monitor for fluctuations indicative of a change in biologically active myelin (i.e., F-wave and distal latencies, amplitude or presence or absence of a response).

In certain embodiments, a method of monitoring treatment following rAAV.hGALC administration is provided wherein the subject is evaluated for a delay in vision loss or absence of vision loss for those subjects that have not developed significant vision loss prior to treatment. Measurement of visual evoked potentials (VEPs) is therefore used to objectively measure responses to visual stimuli as an indicator of central visual impairment or loss. In certain embodiments, the subject is monitored for hearing loss following treatment using, for example, brainstem auditory evoked response (BAER) testing. In certain embodiments, a method of monitoring treatment following rAAV.hGALC administration is provided wherein a subject's psychosine levels are measured.

It is to be noted that the term “a” or “an” refers to one or more. As such, the terms “a” (or “an”), “one or more”, and “at least one” are used interchangeably herein.

The words “comprise”, “comprises”, “comprising”, “containing”, and “including” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%) from the reference given, unless otherwise specified.

As used herein, “disease”, “disorder”, and “condition” are used interchangeably, to indicate an abnormal state in a subject.

The term “expression” is used herein in its broadest meaning and comprises the production of RNA or of RNA and protein. With respect to RNA, the term “expression” or “translation” relates in particular to the production of peptides or proteins. Expression may be transient or may be stable.

As used herein, an “expression cassette” refers to a nucleic acid molecule which comprises a coding sequence, promoter, and may include other regulatory sequences therefor. In certain embodiments, a vector genome may contain two or more expression cassettes. In other embodiments, the term “transgene” may be used interchangeably with “expression cassette”. Typically, such an expression cassette for generating a viral vector contains the coding sequence for the gene product described herein flanked by packaging signals of the viral genome and other expression control sequences such as those described herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein, an “effective amount” refers to the amount of the rAAV composition which delivers and expresses in the target cells an amount of the gene product from the vector genome. An effective amount may be determined based on an animal model, rather than a human patient. Examples of a suitable murine model are described herein.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The following examples are illustrative only and are not intended to limit the present invention.

| Abbreviation | Description |
|---|---|
| A | Absorbance |
| aa | Amino Acids |
| AAV | Adeno-Associated Virus |
| AAVhu68 | Adeno-Associated Virus Serotype hu68 |
| Ad5 | Adenovirus Serotype 5 |
| AE | Adverse Events |
| AEX | Anion Exchange |
| AmpR | Ampicillin Resistance (gene) |
| ANOVA | Analysis of Variance |
| AUC | Analytical Ultracentrifugation |
| BA | Chicken β-Actin Promoter |
| BAER | Brainstem Auditory Evoked Response |
| BBB | Blood-Brain Barrier |
| BCA | Bicinchoninic Acid |
| BDS | Bulk Drug Substance |
| BMCB | Bacterial Master Cell Bank |
| bp | Base Pairs |
| BRF | Batch Record Form |
| BSA | Bovine Serum Albumin |
| BSC | Biological Safety Cabinet |
| BWCB | Bacterial Working Cell Bank |
| cap | Capsid (gene) |
| CBC | Complete Blood Count |
| CBER | Center for Biologies Evaluation and Research |
| CFR | Code of Federal Regulations |
| CFU | Colony Forming Units |
| CI | Chimeric Intron |
| CMC | Chemistry Manufacturing and Controls |
| CMO | Contract Manufacturing Organization |
| CMV IE | Cytomegalovirus Immediate-Early Enhancer |
| CNS | Central Nervous System |
| COA | Certificate of Analysis |
| CRL | Charles River Laboratories |
| CRO | Contract Research Organization |
| CSF | Cerebrospinal Fluid |
| CT | Computed Tomography |
| CTL | Cytotoxic T Lymphocyte |
| ddPCR | Droplet Digital Polymerase Chain Reaction |
| DLS | Dynamic Light Scattering |
| DMEM | Dulbecco's Modified Eagle Medium |
| DMF | Drug Master File |
| DNA | Deoxyribonucleic Acid |
| DO | Dissolved Oxygen |
| DP | Drug Product |
| DRG | Dorsal Root Ganglia |
| DS | Drug Substance |
| DSMB | Data and Safety Monitoring Board |
| E1A | Early Region 1A (gene) |
| ECG | Electrocardiogram |
| EDTA | Ethylenediaminetetraacetic Acid |
| ELISA | Enzyme-Linked Immunosorbent Assay |
| ELISpot | Enzyme-Linked Immunospot |
| ERT | Enzyme Replacement Therapy |
| EU | Endotoxin Units |
| F | Female |
| F/U | Follow-Up |
| FBS | Fetal Bovine Serum |
| FDA | Food and Drug Administration |
| FDP | Final Drug Product |
| FFB | Final Formulation Buffer |
| FIH | First-in-Human |
| GALC | Galactosylceramidase (gene, human) |
| Galc | Galactosylceramidase (gene, mouse) |
| GALC | Galactosylceramidase (protein) |
| GC | Genome Copies |
| GLP | Good Laboratory Practice |
| GMP | Good Manufacturing Practice |
| HCDNA | Host Cell Deoxyribonucleic Acid |
| HCP | Host Cell Protein |
| HEK293 | Human Embryonic Kidney 293 |
| ICH | International Conference on Harmonization |
| ICM | Intra-Cisterna Magna |
| ICV | Intracerebroventricular |
| IDS | Iduronate-2-Sulfatase |
| IFN-γ | Interferon Gamma |
| IT | Intrathecally |
| ITFFB | Intrathecal Final Formulation Buffer |
| ITR | Inverted Terminal Repeat |
| IU | Infectious Unit |
| IV | Intravenous |
| KanR | Kanamycin Resistance (gene) |
| LAL | Limulus Amoebocyte Lysate |
| LFTs | Liver Function Tests |
| LOD | Limit of Detection |
| LTFU | Long-Term Follow-Up |
| M | Male |
| MBR | Master Batch Record |
| MCB | Master Cell Bank |
| MED | Minimum Effective Dose |
| MRI | Magnetic Resonance Imaging |
| mRNA | Messenger Ribonucleic Acid |
| MS | Mass Spectrometry |
| MTD | Maximum Tolerated Dose |
| N | Number of Subjects or Animals |
| N/A | Not Applicable |
| NAbs | Neutralizing Antibodies |
| NBS | Newborn Screening |
| NCV | Nerve Conduction Velocity |
| NGS | Next-Generation Sequencing |
| NHP | Non-Human Primate |
| NHS | Natural History Study |
| OL | Open-Label |
| PAS | Periodic Acid-Schiff |
| PBS | Phosphate-Buffered Saline |
| PEI | Polyethylenimine |
| PES | Polyethersulfone |
| PND | Postnatal Day |
| POC | Proof-of-Concept |
| PolyA | Polyadenylation |
| QA | Quality Assurance |
| QC | Quality Control |
| qPCR | Quantitative Polymerase Chain Reaction |
| rAAV | Recombinant Adeno-Associated Virus |
| rcAAV | Replication-Competent Adeno-Associated Virus |
| rBG | Rabbit β-Globin |
| rDNA | Ribosomal Deoxyribonucleic Acid |
| rep | Replicase (gene) |
| RNA | Ribonucleic Acid |
| RPM | Revolutions Per Minute |
| SA | Single Arm |
| SAE | Serious Adverse Events |
| SD | Standard Deviation |
| SDS | Sodium Dodecyl Sulfate |
| SDS-PAGE | Sodium Dodecyl Sulfate Polyacrylamide Gel |
| | Electrophoresis |
| SOP | Standard Operating Procedure |
| SRT | Safety Review Trigger |
| ssDNA | Single-Stranded Deoxyribonucleic Acid |
| TBD | To Be Determined |
| TCID₅₀ | 50% Tissue Culture Infective Dose |
| TE | Tris-EDTA |
| TFF | Tangential Flow Filtration |
| twi | Twitcher Loss-of-Function Allele |
| UPLC | Ultra-Performance Liquid Chromatography |
| US | United States |
| USP | United States Pharmacopeia |
| VEP | Visual Evoked Potential |
| VP1 | Viral Protein 1 |
| VP2 | Viral Protein 2 |
| VUS | Variants of Unknown Significance |
| WCB | Working Cell Bank |
| WHO | World Health Organization |
| WT | Wild Type |

### Example 1—Recombinant AAVhu68.hGALC

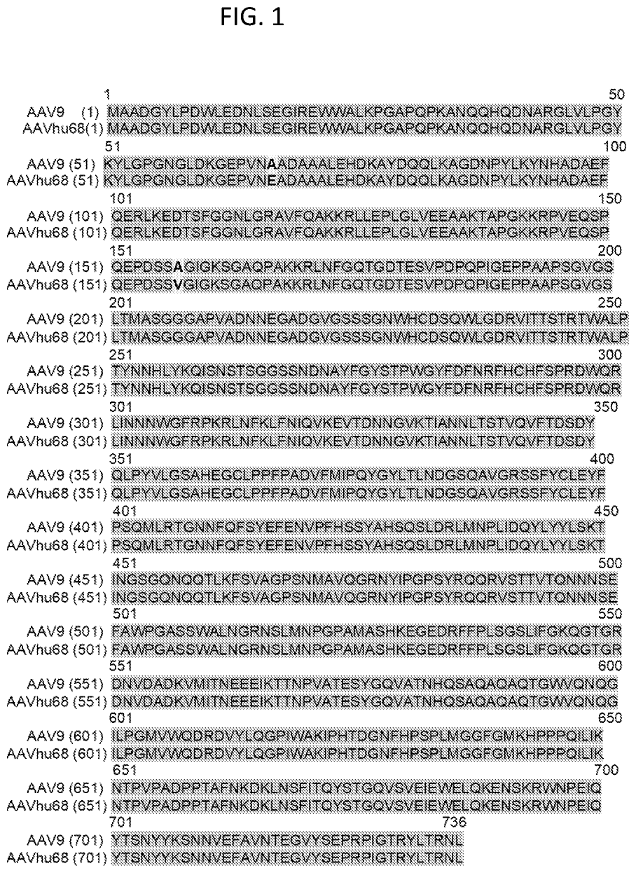

rAAVhu68.hGALC is an AAV that carries an engineered sequence encoding a human GALC. The AAVhu68 capsid of rAAVhu68.hGALC is 99% identical at the amino acid level to AAV9. The two amino acids that differ between the AAV9 [SEQ ID NO: 4] and AAVhu68 capsids [SEQ ID NO: 2] are located in the VP1 (67 and 157) and VP2 (157) regions of the capsid and are identifies in FIG. 1. See also WO 2018/160852, which is incorporated herein by reference.

rAAVhu68.hGALC is produced by triple plasmid transfection of HEK293 cells with an AAV cis plasmid encoding the transgene cassette flanked by AAV ITRs, the AAV trans plasmid encoding the AAV2 rep and AAVhu68 cap genes (pAAV2/hu68.KanR), and the helper adenovirus plasmid (pAdAF6.KanR).

A. AAV Vector Genome Plasmid Sequence Elements

A linear map of the vector genome is shown in FIG. 2. The vector genome contains the following sequence elements:

Inverted Terminal Repeat (ITR): The ITRs are identical, reverse complementary sequences derived from AAV2 (130 bp, GenBank: NC_001401) that flank all components of the vector genome. The ITRs function as both the origin of vector DNA replication and the packaging signal for the vector genome when AAV and adenovirus helper functions are provided in trans. As such, the ITR sequences represent the only cis sequences required for vector genome replication and packaging.

Human Cytomegalovirus Immediate-Early Enhancer (CMV IE): This enhancer sequence obtained from human-derived CMV (382 bp, GenBank: K03104.1) increases expression of downstream transgenes.

Chicken β-Actin Promoter (BA): This ubiquitous promoter (282 bp, GenBank: X00182.1) was selected to drive transgene expression in any CNS cell type.

Chimeric Intron (CI): The hybrid intron consists of a chicken β-actin splice donor (973 bp, GenBank: X00182.1) and rabbit β-globin splice acceptor element. The intron is transcribed, but removed from the mature mRNA by splicing, bringing together the sequences on either side of it. The presence of an intron in an expression cassette has been shown to facilitate the transport of mRNA from the nucleus to the cytoplasm, thus enhancing the accumulation of the steady level of mRNA for translation. This is a common feature in gene vectors intended for increased levels of gene expression.

Coding sequence: An engineered cDNA of the human GALC gene encodes human galactosylceramidase protein, which is a lysosomal enzyme responsible for the hydrolysis and degradation of myelin galactolipids (2055 bp; 685 amino acids [aa], GenBank: EAW81361.1).

Rabbit β-Globin Polyadenylation Signal (rBG PolyA): The rBG PolyA signal (127 bp, GenBank: V00882.1) facilitates efficient polyadenylation of the transgene mRNA in cis. This element functions as a signal for transcriptional termination, a specific cleavage event at the 3′ end of the nascent transcript and the addition of a long polyadenyl tail.

B. Trans Plasmid: pAAV2/1.KanR (p0068)

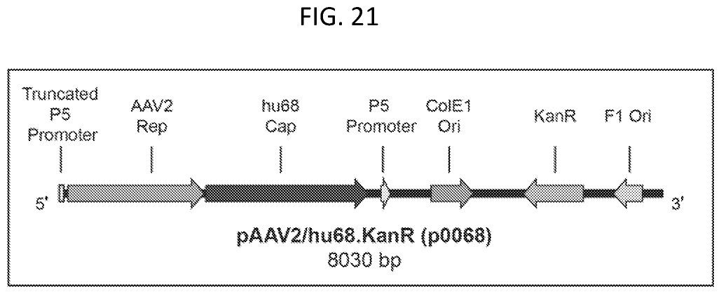

The AAV2/hu68 trans plasmid pAAV2/hu68.KanR (p0068) is presented in FIG. 21.

The AAV2/hu68 trans plasmid is pAAV2/hu68.KanR (p0068). The pAAV2/hu68.KanR plasmid is 8030 bp in length and encodes four wild type AAV2 replicase (Rep) proteins required for the replication and packaging of the AAV vector genome. The pAAV2/hu68.KanR plasmid also encodes three WT AAVhu68 virion protein capsid (Cap) proteins, which assemble into a virion shell of the AAV serotype hu68 to house the AAV vector genome.

To create the pAAV2/hu68.KanR trans plasmid, the AAV9 cap gene from plasmid pAAV2/9n (p0061-2) (which encodes the wild type AAV2 rep and AAV9 cap genes on a plasmid backbone derived from the pBluescript KS vector) was removed and replaced with the AAVhu68 cap gene. The ampicillin resistance (AmpR) gene was also replaced with the kanamycin resistance (KanR) gene, yielding pAAV2/hu68.KanR (p0068). This cloning strategy relocated the AAV p5 promoter sequence (which normally drives rep expression) from the 5′ end of rep to the 3′ end of cap, leaving behind a truncated p5 promoter upstream of rep. This truncated promoter serves to down-regulate expression of rep and, consequently, maximize vector production (FIG. 21).

All component parts of the plasmid have been verified by direct sequencing.

C. Adenovirus Helper Plasmid: pAdDeltaF6(KanR)

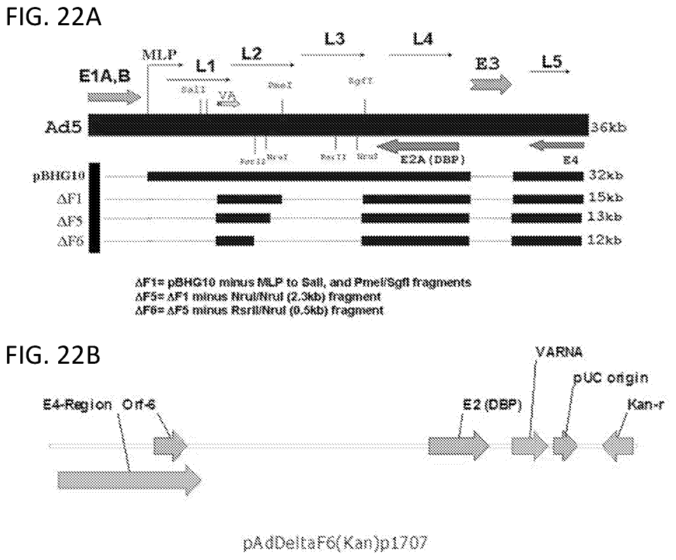

The adenovirus helper plasmid pAdDeltaF6(KanR) is presented in (FIG. 22B)

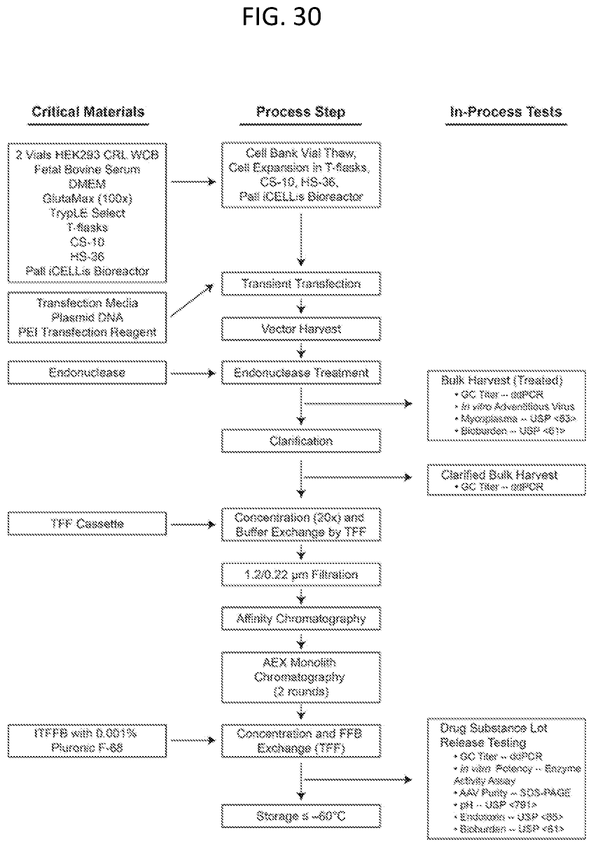

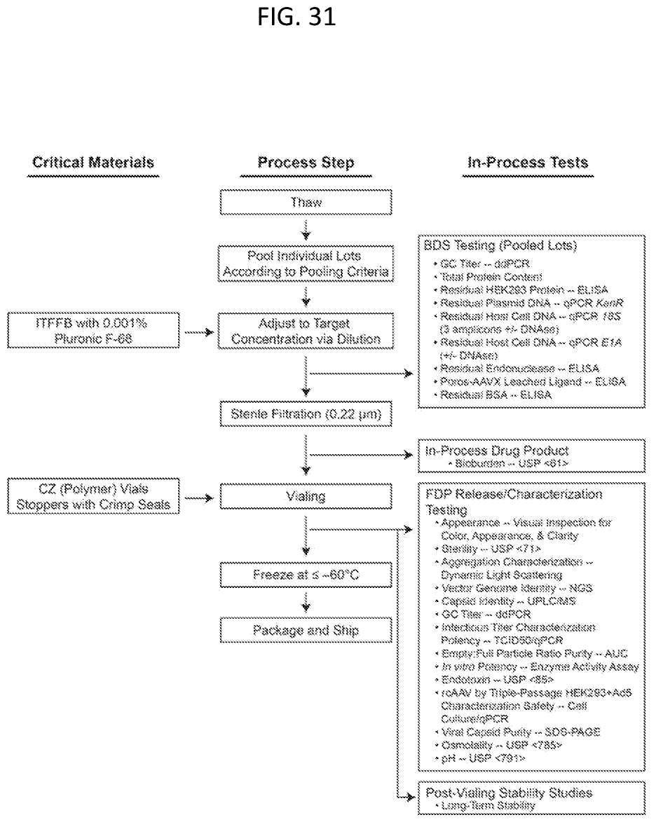

Plasmid pAdDeltaF6(KanR) is 15,770 bp in size. The plasmid contains the regions of adenovirus genome that are important for AAV replication; namely, E2A, E4, and VA RNA (the adenovirus El functions are provided by the HEK293 cells). However, the plasmid does not contain other adenovirus replication or structural genes. The plasmid does not contain the cis elements critical for replication, such as the adenoviral ITRs; therefore, no infectious adenovirus is expected to be generated. The plasmid was derived from an El, E3-deleted molecular clone of Ad5 (pBHG10, a pBR322-based plasmid). Deletions were introduced into Ad5 to eliminate expression of unnecessary adenovirus genes and reduce the amount of adenovirus DNA from 32 kb to 12 kb (FIG. 22A). Finally, the ampicillin resistance gene was replaced by the kanamycin resistance gene to create pAdeltaF6(KanR) (FIG. 22B). The E2, E4, and VAI adenoviral genes that remain in this plasmid, along with El, which is present in HEK293 cells, are necessary for AAV vector production. Vector is produced and formulations are prepared according to the flow charts shown in FIG. 30 and FIG. 31, respectively.

### Example 2—AAVhu68.hGALC Delivery in the GALC-Deficient Twitcher Mouse Model

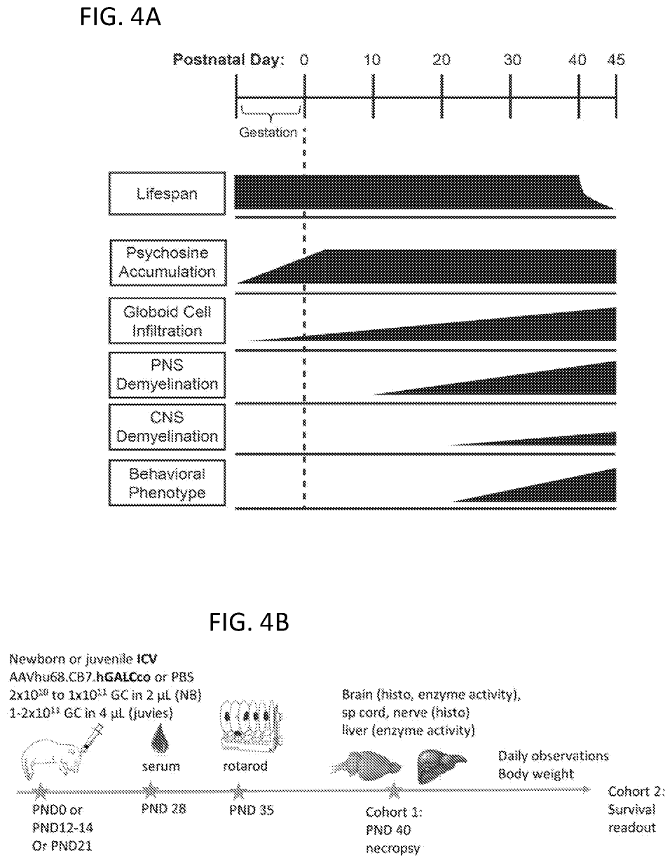

The studies described below used the Twitcher mouse model to establish the potential for delivery of an rAAVhu68 vector (FIG. 2) encoding an engineered human GALC sequence (SEQ ID NO: 9) into the CSF to achieve therapeutic levels of GALC expression levels and rescue several biomarkers of the disease. An overview of the Twitcher mouse studies is provided in FIG. 4B.

The Twitcher mouse is a naturally occurring inbred model of Krabbe disease that was identified as a spontaneous mutation at the Jackson Laboratory in 1976 (Kobayashi T., et al. (1980) Brain Research. 202(2):479-483). Affected mice are homozygous for the twitcher loss-of-function allele (twi), which consists of a G to A mutation in the Galc gene. This mutation causes an early stop codon (W339X). The truncated GALC protein has residual enzymatic activity close to 0%, which is similar to GALC activity levels observed in patients with the infantile form of Krabbe disease. Heterozygous carrier mice (twi/+) are phenotypically normal.

The progression of disease in the Twitcher mouse is well-documented (FIG. 4A), and a variety of neuropathological and behavioral defects phenocopy infantile Krabbe disease as presented in Table 5. As in infantile Krabbe patients, GALC deficiency in mice results in the accumulation of the cytotoxic lipid intermediate, psychosine. Twitcher mice likewise display massive infiltration of PNS and CNS white matter by phagocytic, psychosine-filled globoid cells, which are thought to be derived from macrophage and/or microglial lineages (Tanaka K., et al. (1988) Brain Research. 454(1):340-346; Levine S. M., et al. (1994) Intl J Dev Neuro. 12(4):275-288). This results in demyelination, which is one of the key hallmarks of disease in Krabbe patient. After an initial period of normal myelination, affected Twitcher mice lose myelin after 10 days of age in the PNS due to the death of myelin-forming Schwann cells (Jacobs J. M., et al. (1982) J Neurol Sci. 55(3):285-304) and 20 days of age in the CNS due to the death of myelin-forming oligodendrocytes. It is likely because of this delay that demyelination is more severe in the peripheral nerves than in the CNS of these mice (Suzuki K. & Suzuki K. (1983) The American journal of pathology. 111(3):394-397). Finally, Twitcher mice display consistent and rapid neurological deterioration after the onset of symptoms, which is similarly observed in infantile Krabbe patients upon symptom onset. Behavioral symptoms in these mice include motor phenotypes reminiscent of those observed in human patients, including tremors, twitching, and hind leg weakness, which present at approximately 20 days of age. Mice ultimately progress to a humane endpoint characterized by severe weight loss and paralysis by around 40 days of age (Wenger D. A. (2000) Molec Med Today. 6(11):449-451).

Infantile Krabbe patients exhibit similar clinical features as the Twitcher mice. Thus, the Twitcher mouse model is adequate to assess the efficacy (rescue of enzyme activity to improve survival, motor function, and brain and nerve pathology) of rAAVhu68.hGALC to support an infantile Krabbe indication. Studies using the Twitcher mouse, described below, demonstrated the efficacy of rAAVhu68.hGALC to express active GALC enzyme in the relevant tissues, rescue survival, ameliorate motor function, and ameliorate CNS and PNS histopathology after a single ICV administration (the most efficient route of administration in mouse models where ICM is not technically feasible).

### Presymptomatic Newborn Twitcher Mice

The aim of this study was to establish the optimal ROA, capsid serotype, and dose range for achieving maximal efficacy in the Twitcher mouse model. Presymptomatic newborn mice were selected for these studies in order to maximize the change of observing disease rescue.

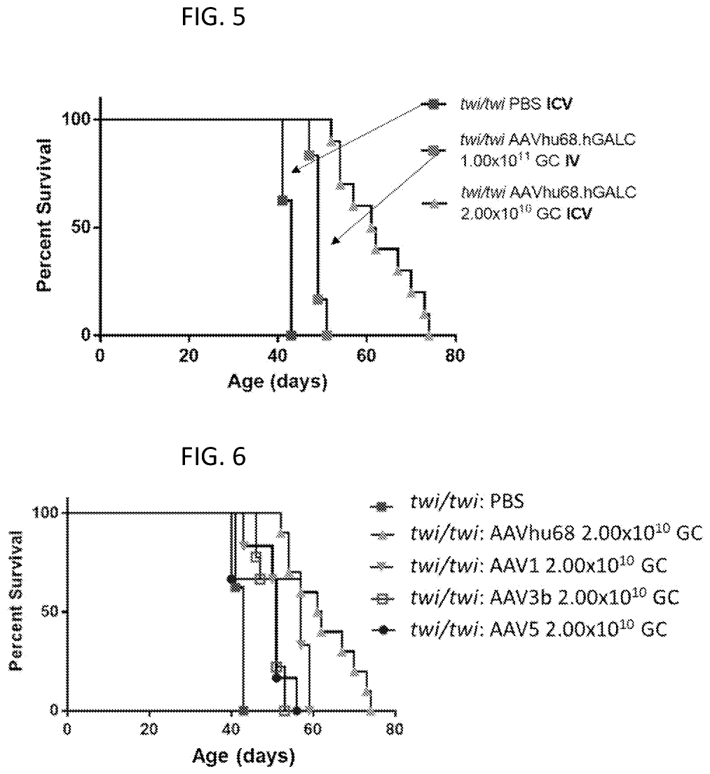

To establish the optimal ROA, the IV route via injection into the temporal vein was compared to the ICV route because both routes can transduce the CNS and PNS in newborn animals. Presymptomatic Twitcher mice (twi/twi) were administered rAAVhu68.hGALC either IV at a dose of 1.00×10¹¹ GC or ICV at a 5-fold lower dose of 2.00×10¹⁰ GC on PND 0. The IV dose was selected because it corresponds to 1.00×10¹⁴ GC/kg, a high dose needed to achieve CNS transduction, and a 5-fold lower ICV dose was selected because direct administration into the CSF facilitates CNS transduction at lower doses. As a control, presymptomic age-matched Twitcher mice were injected ICV with vehicle (PBS). Animals were euthanized upon reaching a humane endpoint defined by weight loss >20% of maximal body weight and/or hind leg paralysis, and survival was recorded. IV administration of rAAVhu68.hGALC at the higher dose (1.00×10¹¹ GC) provided some survival benefit compared to the untreated controls. However, compared to IV administration, ICV-administered rAAVhu68.hGALC at a 5-fold lower dose (2.00×10¹⁰ GC) conferred a superior survival benefit (FIG. 5). The ICV ROA was therefore selected for subsequent studies.

To identify the best AAV capsid for nervous system delivery of a vector genome encoding human GALC, four different AAV capsids were tested. The capsids included AAV serotype 3b (AAV3b), AAV serotype 5 (AAV5), AAV serotype 1 (AAV1), and AAV serotype hu68 (rAAVhu68.hGALC). Each AAV vector was administered ICV at a dose of 2.00×10¹⁰ GC, which was the low dose and ROA previously established to effectively prolong survival in presymptomatic Twitcher mice while allowing a short study duration (FIG. 5). As a control, a group of presymptomatic Twitcher mice were ICV-administered vehicle only (PBS) on PND 0. While all four capsids enhanced survival compared to the vehicle-treated controls, the AAVhu68 capsid (rAAVhu68.hGALC) yielded superior survival over AAV3b, AAV5, and AAV1 (FIG. 6). The AAVhu68 capsid (rAAVhu68.hGALC) was therefore selected for subsequent studies.

To determine the dose range, rAAVhu68.hGALC was ICV-administered at a dose of 2.00×10¹⁰ GC, 5.00×10¹⁰ GC, or 1.00×10¹¹ GC to newborn presymptomatic Twitcher mice on PND 0. As controls, age-matched presymptomatic Twitcher (twi/twi) mice and unaffected heterozygotes (twi/+) and wild type mice were ICV-administered vehicle (PBS) on PND 0.

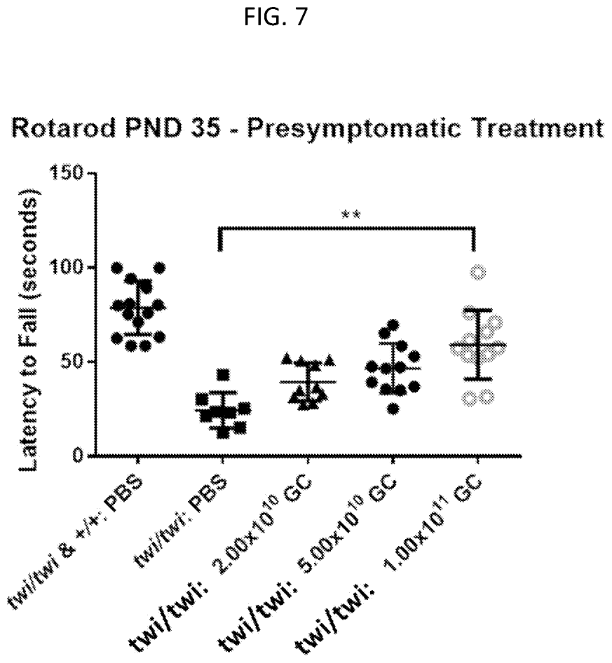

At PND 35, the mobility and coordination of the mice were assessed using a rod assay (FIG. 7). The rotarod is an accelerating rod on which mice run, and the time latency between the initiation of the assay and the point at which mice fall off the rod is well-established to correlate with motor coordination. The PND 35 time point was selected because it is a time point at which affected Twitcher mice display measurable motor deficits prior to reaching a humane euthanasia endpoint. The rotarod assay revealed partial rescue of motor and coordination following rAAVhu68.hGALC administration. The extent of rescue appeared to be dose-dependent, and significantly longer fall latencies were observed for the highest rAAVhu68.hGALC dose of 1.00×10¹¹ GC (p<0.01) (FIG. 7).

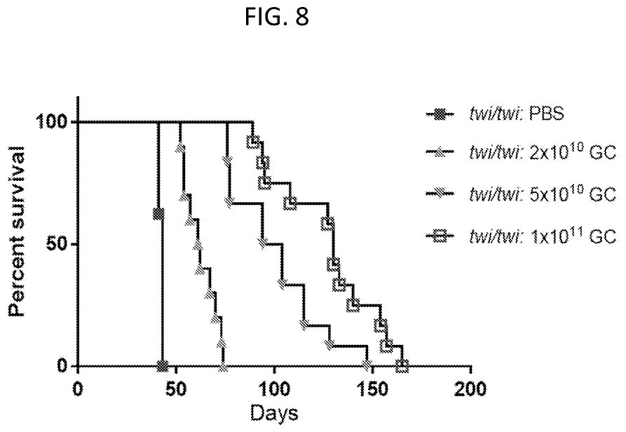

Following the rotarod assay, survival was tracked for all treatment groups. A dose-dependent increase in survival was observed for Twitcher (twi/twi) mice administered rAAVhu68.hGALC presymptomatically on PND 0 compared to the vehicle-treated Twitcher (twi/twi) controls. The longest median survival (130 days) was observed for mice administered the highest rAAVhu68.hGALC dose of 1.00×10¹¹ GC (FIG. 8).

Cumulatively, these POC experiments to identify the optimal ROA, capsid, and dose range demonstrate the efficacy of rAAVhu68.hGALC at preserving neuromotor function and prolonging survival if administered prior to symptom onset in Twitcher mice.

### Symptomatic Twitcher Mice

The aim of this study was to examine the efficacy of rAAVhu68.hGALC when administered during the early phase of disease pathology prior to the onset of behavioral symptoms (PND 12; referred to as “early-symptomatic”) or during a later phase of disease pathology when mice display behavioral symptoms (PND 21; referred to as “later-symptomatic”) because we wanted to recapitulate a context similar to patients enrolled after symptom onset. Moreover, PND 0 mice have a brain maturation equivalent to a pre-term fetus, whereas PND 12 and PND 21 translate to a 2-month-old and a 9-month-old, respectively (www.translatingtime.org), which more closely recapitulates the intended infantile population for the FIH.

Early-symptomatic Twitcher mice were ICV-administered rAAVhu68.hGALC at a dose of either 1.00×10¹¹ GC or 2.00×10¹¹ GC on PND 12, while another cohort of later-symptomatic Twitcher mice were ICV-administered rAAVhu68.hGALC at a higher dose of 2.00×10¹¹ GC on PND 21. The lower dose of 1.00×10¹¹ GC was selected for administration because it was found to be the most effective dose at increasing survival of Twitcher mice (described above). A higher rAAVhu68.hGALC dose of 2.00×10¹¹ GC was also utilized for the treatment of mice on PND 21 because it was hypothesized that a higher dose might be needed for mice with more severe demyelination. As controls, early-symptomatic Twitcher mice (twi/twi) and unaffected heterozygotes (twi/+) were ICV-administered vehicle only (PBS) on PND 12, and historic data from Study 1 were used to compare with Twitcher mice (twi/twi) administered 1.11×10¹¹ GC at PND 0.

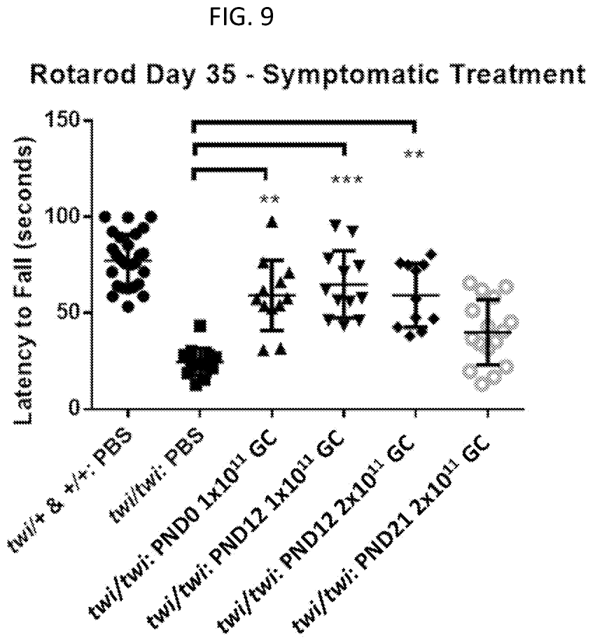

At PND 35, the mobility and coordination of the mice were assessed using the rotarod assay, which is a time point at which Twitcher mice (twi/twi) display measurable motor deficits. The rotarod assay revealed partial rescue of motor and coordination when rAAVhu68.hGALC was administered to pre-symptomatic Twitcher mice on PND 0 at a dose of 1.00×10¹¹ GC (p<0.01) or to early-symptomatic Twitcher mice on PND 12 at a dose of either 1.00×10¹¹ GC (p<0.001) or 2.00×10¹¹ GC (p<0.01). No significant rescue of motor and coordination was observed when a rAAVhu68.hGALC dose of 2.00×10¹¹ GC was administered to later-symptomatic Twitcher mice on PND 21 (FIG. 9).

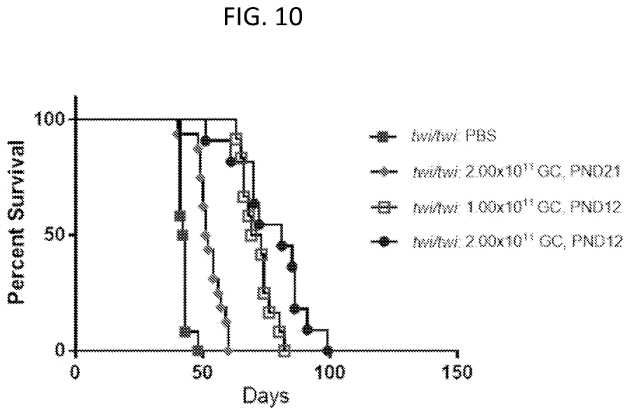

Following the rotarod assay, survival was tracked for all treatment groups. Compared to vehicle-treated Twitcher controls, longer survival was observed following rAAVhu68.hGALC administration in both early-symptomatic Twitcher mice on PND 12 and later-symptomatic Twitcher mice on PND 21. However, maximal survival was obtained when early-symptomatic Twitcher mice were administered rAAVhu68.hGALC at the high dose of 2.00×10¹¹ GC on PND 12 (FIG. 10).

Cumulatively, improvements in survival and neuromotor function of Twitcher mice suggested that rAAVhu68.hGALC may be more efficacious if administered at earlier stages of disease.

### Pharmacology Study in Symptomatic Twitcher Mice

The aim of this study was to assess pharmacology, functional, and histopathology readouts after rAAVhu68.hGALC administration. Because previous studies sacrificed animals at a humane endpoint in order to faciliate survival anlyses, this precluded the collection of pharmacology and histology readouts at age-matched time points in Twitcher mice and vehicle-treated controls. Therefore, this study examined these endpoints.

Early-symptomatic Twitcher mice (twi/twi) were ICV-administered rAAVhu68.hGALC at a dose of 2.00×10¹¹ GC on PND 12. Age-matched unaffected Twitcher heterozygotes (twi/+) and wild-type mice were ICV-adminstered PBS as controls on PND 12. A rAAVhu68.hGALC dose of 2.00×10¹¹ GC was selected for POC to achieve maximal efficacy, and PND 12 was selected as the day of dosing because it is shortly after the onset of PNS demyelination in an animal with brain maturation equivalent to a 2-month-old infant (www.translatingtime.org), which mirrors the intended infantile population for the FIH trial.

Beginning on PND 22, all mice were monitored daily for clinical signs. PND 22 was selected as the first time point for this assessment because this is one of the earliest days at which behavior phenotypes are observable in Twitcher mice. Clinical signs were scored using an unpublished assessment of clasping ability, gait, tremor, kyphosis, and fur quality as detailed in Table 1. These measures effectively assess the clinical status of Twitcher mice based upon the symptoms they typically present. Scores above 0 indicate clinical deterioration.

TABLE 1
Clinical Scoring Assessment of Mice

| Assessment | | |
|---|---|---|
| Category | Observation | Score |
| Hind Limb | No clasping | 0 |
| Clasping | Non-permanent clasping | 1 |
| | Permanent Clasping | 2 |
| Gait | Normal | 0 |
| | Slightly abnormal gait but mouse | 1 |
| | moves easily and spontaneously | |
| | Markedly abnormal gait, reduced | 2 |
| | spontaneous mobility | |
| | Severe difficulty moving forward, | 3 |
| | dragging of hind legs | |
| Tremors | Normal | 0 |
| | Minimal tremors, only visible | 1 |
| | when mice are immobile | |
| | Moderate tremors, noticeable at | 2 |
| | rest and while moving. Twitching | |
| | Marked tremors and twitching, obvious | 3 |
| | both at rest and while moving. | |
| Spinal Curvature | Normal | 0 |
| | Mild kyphosis (curved spine), but | 1 |
| | able to straighten spine completely | |
| | Unable to straighten spine completely; | 2 |
| | maintenance of persistent mild kyphosis | |
| | Maintenance of pronounced kyphosis | 3 |
| | while walking or sitting | |
| Fur Quality | Normal | 0 |
| | Any abnormality (scruffed, | 1 |
| | alopecia, etc.) | |

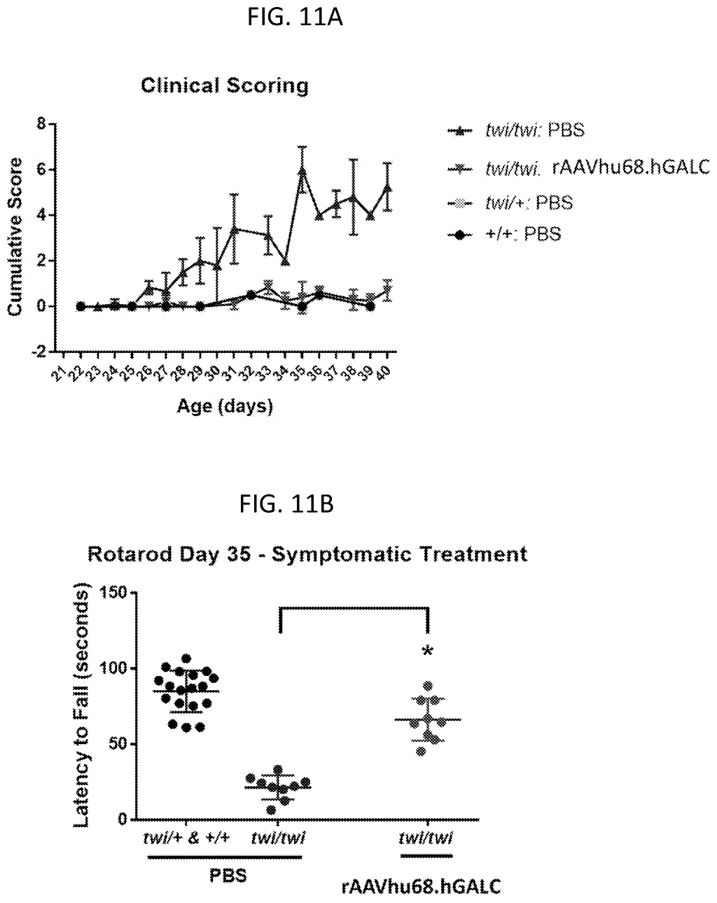

Using this assessment, early-symptomatic Twitcher mice (twi/twi) administered rAAVhu68.hGALC on PND 12 displayed clinical scores close to 0, which was comparable to the scores of wild-type and unaffected Twitcher heterozygotes (twi/+). In constrast, the age-matched vehicle-treated Twitcher (twi/twi) mice displayed higher assessment scores over most of the time course, indicating clinical deterioration (FIG. 11A).

As a complementary functional assay, the rotarod test was performed on PND 35 to evaluate neuromotor phenotypes. Early-symptomatic Twitcher mice (twi/twi) administered rAAVhu68.hGALC on PND 12 displayed fall latencies comparable to those of the wild-type and unaffected Twitcher heterozygotes (twi/+), while the age-matched vehicle-treated Twitcher mice (twi/twi) displayed significantly shorter fall latencies (p<0.05), indicating deterioration of neuromotor function (FIG. 11B).

To determine whether the observed benefits of rAAVhu68.hGALC administration on functional endpoints correlated with histologic improvements, all mice were necropsied on PND 40, and the sciatic nerve of the hind limb was examined histologically. PND 40 was selected as the necropsy time point because it is the approximate age when untreated or vehicle-treated Twitcher mice reach humane endpoint and it is a time point at which the neuropathology is most severe. The sciatic nerve was selected for histology because it peripheral nerves are more affected by demyelination in Twitcher mice compared to the CNS. Furthermore, lack of correction of the PNS by HSCT in patients remains a major unmet neet in infantile patients, so an examination of the effect of rAAVhu68.hGALC administration on the PNS is of interest.

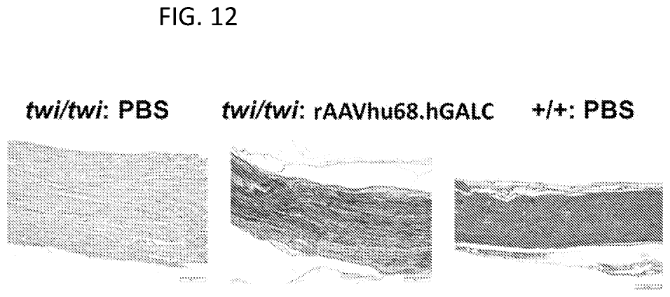

Sections of the sciatic nerves were processed for visualization of myelin (dark staining) and globoid cells (light staining) (FIG. 12). On PND 40, the sciatic nerve of vehicle-treated wild type controls was enriched with myelin and generally devoid of globoid cell infiltrates. However, in vehicle-treated symptomatic Twitcher mice (twi/twi), severe subtotal demyelination was observed in the sciatic nerve, accompanied by nerve thickening and the infiltration of globoid cells. In contrast, myelin was preserved in the sciatic nerve of symptomatic Twitcher mice (twi/twi) administered rAAVhu68.hGALC on PND 12, although not to the extent normally observed in age-matched wild-type mice. Fewer globoid cells were also observed in the nerve of rAAVhu68.hGALC-treated Twitcher mice compared to vehicle-treated Twitcher mice. These data correlated with the functional endpoints, suggesting that improved clinical scores and neuromotor function in symptomatic Twitcher mice administered rAAVhu68.hGALC during the early phase of disease progression may be related to either a reversal or delay in onset of the underlying disease neuropathology.

Finally, samples of brain, liver, and serum were obtained from wild type and Twitcher mice (twi/twi) on the day of necropsy (PND 40) to quantify activity levels of the transgene product, GALC. GALC was quantified using a fluorophore-based GALC activity assay to confirm that following rAAVhu68.hGALC administration, the AAV vector was transduced and a functional enzyme was expressed. Wild type animals were used as the control for this assay, and Twitcher heterozygotes (twi/+) were excluded because GALC activity levels are reduced in those mice despite having no observable phenotype. The brain was examined because the nervous system is the target tissue for GALC delivery, and the liver and serum were examined to assess transduction in peripherial organ systems.

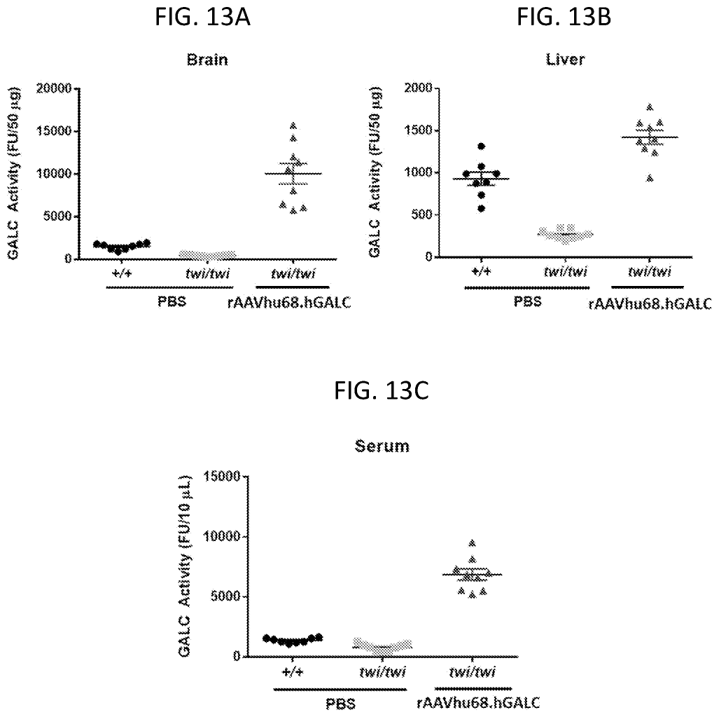

Twenty-eight days after ICV administration of rAAVhu68.hGALC at a dose of 2.00×10¹¹ GC, supraphysiologic levels of GALC activity were observed in the brain, liver, and serum of Twitcher (twi/twi), which were higher than the levels observed in the same tissues of vehicle-treated Twitcher (twi/twi) mice and wild type controls (FIG. 13A-FIG. 13C).

Cumulatively, the data demonstrated that the administration of rAAVhu68.hGALC to early-symptomatic Twitcher mice results in supraphysiologic levels of GALC activity that correlates with less severe clinical symptoms, neuromotor dysfunction, PNS demyelination, and globoid cell neuropathology.

Effect of Bone Marrow Transplant in Combination with rAAVhu68.hGALC Administration

This study investigated the potential benefit of a dual therapy of rAAVhu68.hGALC and bone marrow transplant (BMT). We investigated this combination therapy because of the prominent neuroinflammatory component of Krabbe disease. In theory, there is a synergistic effect because the HSCT provides an additional source of GALC enzyme in the CNS (from macrophage/microglial cells derived from the transplanted cells and rAAVhu68.hGALC-transduced neurons), while rAAVhu68.hGALC provides correction to the PNS, which is not affected by HSCT. Moreover, different combination treatment designs were examined in this study to assess whether rAAVhu68.hGALC might be efficacious in 1) patients who receive a HSCT first through NBS programs followed by gene therapy and/or 2) patients who receive gene therapy first followed by HSCT, if eligible.

The combination therapy study is summarized in Table 2.

TABLE 2
Study of AAV and BMT Combination Therapy in Mice

| | | | Treatment | Time | Treatment | Time | |
|---|---|---|---|---|---|---|---|
| Group | Genotype | N | #1 | Point | #2 | Point | Rationale |
| 1 | twi/twi | 13 | BMT | PND 10 | — | — | Assess efficacy of |
| | | | | | | | BMT monotherapy |
| 2 | twi/twi | 7 | rAAVhu68.hGALC | PND 0 | BMT | PND 10 | Assess efficacy of |
| | | | | | | | rAAVhu68.hGALC |
| | | | | | | | treatment followed |
| | | | | | | | by BMT as early as |
| | | | | | | | possible in |
| | | | | | | | newborn pre- |
| | | | | | | | symptomatic mice |
| 3 | twi/twi | 7 | BMT | PND 10 | rAAVhu68.hGALC | PND 12 | Assess efficacy of |
| | | | | | | | BMT followed by |
| | | | | | | | rAAVhu68.hGALC |
| | | | | | | | treatment; PND 10 |
| | | | | | | | was found to be |
| | | | | | | | earliest time point |
| | | | | | | | for BMT in pilot |
| | | | | | | | experiments |
| | | | | | | | because busulfan |
| | | | | | | | conditioning is |
| | | | | | | | toxic to mice |
| | | | | | | | weighing less than |
| | | | | | | | 4 g |
| 4 | twi/twi | TBD | rAAVhu68.hGALC | PND 12 | BMT | PND 28 | Assess efficacy of |
| | | | | | | | rAAVhu68.hGALC |
| | | | | | | | treatment followed |
| | | | | | | | by BMT in early- |
| | | | | | | | symptomatic mice |
| | | | | | | | at a later stage of |
| | | | | | | | disease progression |
| 5 | twi/twi | 12 | rAAVhu68.hGALC | PND 0* | — | — | Control: |
| | | | | | | | rAAVhu68.hGALC |
| | | | | | | | monotherapy in |
| | | | | | | | pre-symptomatic |
| | | | | | | | mice |
| 6 | twi/twi | 13 | rAAVhu68.hGALC | PND 12* | — | — | Control: |
| | | | | | | | rAAVhu68.hGALC |
| | | | | | | | monotherapy in |
| | | | | | | | early-symptomatic |
| | | | | | | | mice |
| 7 | twi/twi | 12 | PBS | * | — | — | Control: Vehicle- |
| | | | | | | | only treatment |

rAAVhu68.hGALC was administered at a dose of 1.00 × 10¹¹ GC.
All mice receiving a BMT also undergo myeloablative conditioning with busulfan 1-2 days prior to the BMT procedure to reduce the quantity of endogenous bone marrow cells.
*Historical controls are used for these groups. Group 5 is a historical control from Study 1. Group 6 is a historical control from Study 2. Group 7 is a historical control consisting of N = 8 mice from Study 1 (PND 0) and N = 4 mice from Study 2 (PND 12).
Abbreviations: AAV, adeno-associated virus; BMT, bone marrow transplant; GC, genome copies; PBS, phosphate- buffered saline; PND, postnatal day; TBD, to be determined.

The rAAVhu68.hGALC dose of 1.00×10¹¹ GC was utilized because we anticipate a better response due to the combination therapy, which permits a lower dose of rAAVhu68.hGALC than was used in previous studies of rAAVhu68.hGALC monotherapy. Efficacy of rAAVhu68.hGALC is assessed in terms of survival, body weight, and neurologic observations (e.g., presence of tremor and abnormal clasping reflex).

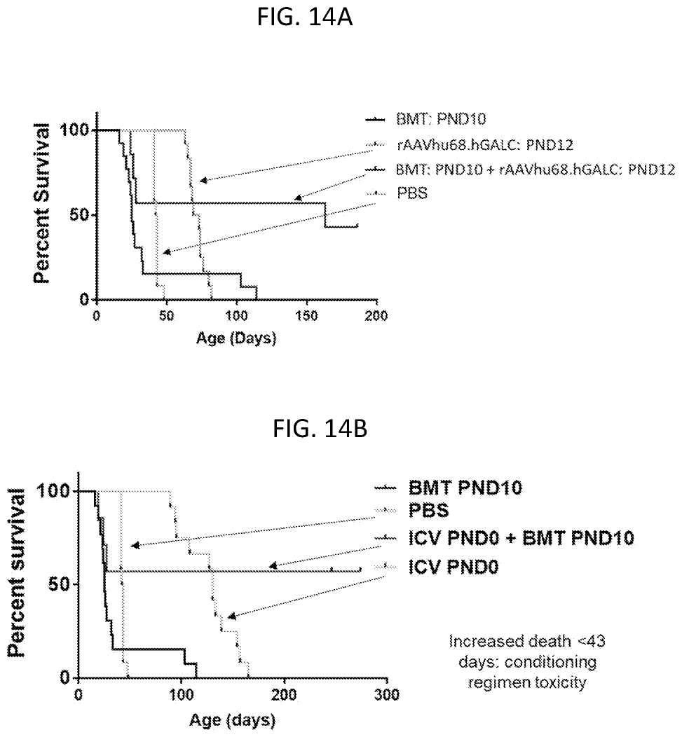

The survival data for Groups 1-3 are shown in FIG. 14A-FIG. 14B. Thus far, the best survival was achieved with the combination of treating presymptomatic Twitcher mice (twi/twi) with ICV-administered rAAVhu68.hGALC on PND 0 followed by BMT on PND 10 (Group 2). Survival was extended to >300 days in the absence of overt signs. These mice appear to be in better physical condition based upon the previously described clinical assessment (Table 1), displaying a slight tremor with no noticeable gait abnormalities and no clasping (analysis still ongoing; data not shown). Mice that received BMT before rAAVhu68.hGALC (Group 3) are currently still alive (N=3/7), but they display marked tremor, some gait abnormality, and lower body weight. However, the busulfan conditioning regimen coupled with BMT is toxic in mice younger than 10 days of age, and mice in both Groups 2 and 3 displayed increased mortality either before or shortly after BMT, regardless of the order of the combination therapies. Group 4 are being injected to mimic a clinically relevant situation of gene therapy administered to early symptomatic patients followed by BMT.

Cumulatively, these data suggest that combining rAAVhu68.hGALC treatment with a subsequent BMT may provide more efficacy than each treatment alone in the murine model of Krabbe disease.

### Example 3—Identification of the Minimum Effective Dose (MED) of rAAVhu68.GALC in the Twitcher Mouse Model

The MED study is performed in the Twitcher mouse using the toxicological vector lot manufactured for a nonhuman primate pharmacology-toxicology study. The study includes at least two timepoints and evaluates four dose levels to determine the MED, pharmacology, and histopathology (efficacy and safety). The dose levels were selected based on the pilot dose range study and the maximal feasible dose when scaled to humans. Animals are injected ICV at PND12 to mimic early symptomatic patients. Some of the animals are sacrificed one-month post-injection (when vehicle treated reach humane endpoint) to obtain pharmacological and efficacy readouts compared to age-matched controls (similar design than study 3). The remaining mice are followed until a humane endpoint to evaluate the effect of treatment on survival. Personnel doing the in-life evaluation (body weight, clinical scoring and rotarod assay) are blinded to the mice treatment and genotype.

MED is determined upon analysis of survival benefit, clinical scoring, body weight, neuromotor function using the rotarod assay, GALC activity levels in target organs, and correction of neuropathology in the CNS and PNS (i.e., improved myelination, decreased globoid cells infiltration).

The study design and study schedule are presented below in Table 3 and Table 4.

TABLE 3
Murine MED Study Design

| | | | | | | Sacrifice: | |
|---|---|---|---|---|---|---|---|
| | | | | | | PND40- | |
| | | Affected | WT | | | 42 Days | Sacrifice: |
| | | Mice | Mice | | Sacrifice: | Post | Humane |
| Group | Treatment | (twi/twi) | (+/+) | In-Life | baseline | Injection | Endpoint |
| # | (Dose) | (N) | (N) | Evaluation | PND12-14 | (N) | (N) |
| 1 | none | 6 | — | none | 6 | N/A | N/A |
| | | | | | (twi/twi); | | |
| 2 | none | — | 6 | | 6 | | |
| | | | | | (+/+) | | |
| 3 | Vehicle (Artificial | 16 | — | Daily | N/A | 8 | 8 |
| | CSF ITFFB) | | | observations | | (twi/twi) | (twi/twi) |
| 4 | Vehicle (Artificial | — | 10 | Body weight | N/A | 5 | 5 |
| | CSF ITFFB) | | | 3x/week | | (+/+) | (+/+) |
| 5 | rAAVhu68.hGALC | 16 | — | Clinical score | N/A | 8 | 8 |
| | 2.00 × 10¹¹ GC | | | 3x/week | | | |
| 6 | rAAVhu68.hGALC | 16 | — | Rotarod on | N/A | 8 | 8 |
| | 6.80 × 10¹⁰ GC | | | PND35-37 | | | |
| 7 | rAAVhu68.hGALC | 16 | — | | N/A | 8 | 8 |
| | 2.00 × 10¹⁰ GC | | | | | | |
| 8 | rAAVhu68.hGALC | 16 | — | | N/A | 8 | 8 |
| | 6.80 × 10⁹ GC | | | | | | |

Abbreviations:
CSF, cerebrospinal fluid;
N, number of animals;
WT, wild type,
ITFFV, intrathecal final formulation buffer (ITFFB)

TABLE 4
Study Schedule

| Study | Sample/ | Group | Group | Group | Group | Group | Group | Group | Group |
|---|---|---|---|---|---|---|---|---|---|
| Day | Procedure | 1 | 2 | 3 | 4 | 5 | 6 | 7 | 8 |
| PND1-PND7 | Microtattoo | Multiple litters | | | | | | | |
| | identification | | | | | | | | |
| | genotyping | Multiple litters | | | | | | | |
| PND12-14 | ICV injection | — | — | 16 | 10 +/+ | 16 | 16 | 16 | 16 |
| | | | | (twi/twi) | | (twi/twi) | (twi/twi) | (twi/twi) | (twi/twi) |
| | Necropsy | 6 | 6 +/+ | — | — | — | — | — | — |
| | | (twi/twi) | | | | | | | |
| PND12-14 - | Daily | — | — | 16 | 10 +/+ | 16 | 16 | 16 | 16 |
| end | observation and | | | (twi/twi) | | (twi/twi) | (twi/twi) | (twi/twi) | (twi/twi) |
| of study | survival | | | | | | | | |
| | monitoring | | | | | | | | |
| PND21-28 | Weaning | — | — | 16 | 10 | 16 | 16 | 16 | 16 |
| | Genotyping | — | — | 16 | 10 | 16 | 16 | 16 | 16 |
| | confirmation | | | | | | | | |
| | Microchip | — | — | 16 | 10 | 16 | 16 | 16 | 16 |
| | identification | | | | | | | | |
| Weaning - | 3 x/week | — | — | 16 | 10 | 16 | 16 | 16 | 16 |
| end of | body weight | | | | | | | | |
| study | and | | | | | | | | |
| | neurological | | | | | | | | |
| PND 35-37 | Rotarod | — | — | 16 | 10 | 16 | 16 | 16 | 16 |
| | Submandibular | — | — | 16 | 10 | 16 | 16 | 16 | 16 |
| | bleed | | | | | | | | |
| PND40-42 | Necropsy | — | — | 8 | 5 | 8 | 8 | 8 | 8 |
| Survival | Necropsy | — | — | 8 | 5 | 8 | 8 | 8 | 8 |
| follow up | humane | | | | | | | | |

### Example 4—Efficacy of AAV-Mediated Gene Therapy to Treat Krabbe Dogs—Injection of rAAVhu68.CB7.CI.cGALCco.rBG Via the Cisterna Magna

While an informative disease model, the Twitcher mouse does have some limitations. The mice display only mild CNS involvement, which is distinct from infantile Krabbe patients, who present with more severe CNS features of demyelination of brain atrophy. Furthermore, the small size of the mouse poses experimental challenges. The ICV route must be used in mice because their small size makes it difficult to reliably inject AAV vector via the intended clinical route (ICM). Sufficient quantities of serial samples of CSF and blood also cannot be obtained from mice for all of the desired pharmacological assays. Treatment with rAAVhu68.GALC was therefore evaluated in a larger animal, the canine model of Krabbe disease, which can overcome these technical constraints and confirm the scalability of our therapeutic approach.

Like the Twitcher mouse, the Krabbe dog is a naturally occurring autosomal recessive disease model deriving from a spontaneous A to C mutation in the GALC gene that causes a missense mutation (Y158S). The mutant GALC protein has residual enzymatic activity close to 0%, which is similar to GALC activity levels observed in patients with the infantile form of Krabbe disease. While heterozygous dogs do not display symptoms, dogs homozygous for the mutation are affected.

Krabbe dogs present with a severe phenotype similar to infantile Krabbe disease as summarized in Table 5. While the progression of the Krabbe dog phenotype is less well characterized than the Twitcher mouse, affected Krabbe dogs display demyelination and globoid cells accumulation that affects both CNS and peripheral nerves. They develop hind limb weakness, thoracic limb dysmetria, and tremors at approximately 4-6 weeks of age. Like infantile Krabbe patients, Krabbe dogs present with a consistent and rapid neurologic deterioration after the onset of symptoms. Ultimately, these symptoms progress to a humane endpoint characterized by severe ataxia, pelvic limb paralysis, wasting, urinary incontinence, and sensory deficits by around 15 weeks of age (Fletcher T. F. & Kurtz H. J. (1972) Am J Pathol. 66(2):375-8; Wenger D. A. (2000) Molec Med Today. 6(11):449-451; Bradbury A. M., et al. (2018) Hum Gene Ther. 29(7):785-801).

TABLE 5
Murine and canine models of Krabbe disease and comparison
with human early infantile presentation

| | | GALC | Presenting | |
|---|---|---|---|---|
| | | Activity | Signs and | |
| | Mutation | Levels | Evolution | Pathology |
| Twitcher | Murine: | <10% of | Twitching, hind leg | PNS > |
| Mouse | G.A1017 | normal | weakness at 18-22 days | CNS |
| | (W339X) | | Severe weight loss and | |
| | | | paralysis at 40-45 days | |
| Krabbe Dog | Canine: | <10% of | Thoracic limb dysmetria, | PNS and |
| | A.C 473 | normal | hind limb weakness, | CNS |
| | (Y158S) | | tremors at | |
| | | | 4-6 weeks | |
| | | | Pelvic limb paralysis, | |
| | | | severe ataxia, wasting, | |
| | | | urinary incontinence, | |
| | | | sensory deficits at about | |
| | | | 15 weeks | |
| Human Early | Various, most | <10% of | Irritability, spasticity, | PNS and |
| Infantile | frequent is a | normal | dysphagia before 6 months | CNS |
| Krabbe | 30 kb deletion | | of age | |
| Disease | starting near | | Regression and absence of | |
| | intron 10 | | new milestones | |
| | (502T/del) | | acquisition, deafness, | |
| | | | blindness, seizures, death | |
| | | | before 2 years | |

Abbreviations: A, adenine; C, cytosine; CNS, central nervous system; G, guanine; GALC, galactosylceramidase; kb, kilobase; PNS, peripheral nervous system; W339X, tryptophan changed to a stop codon at position 339; Y158S, tyrosine to serine substitution at position 158.

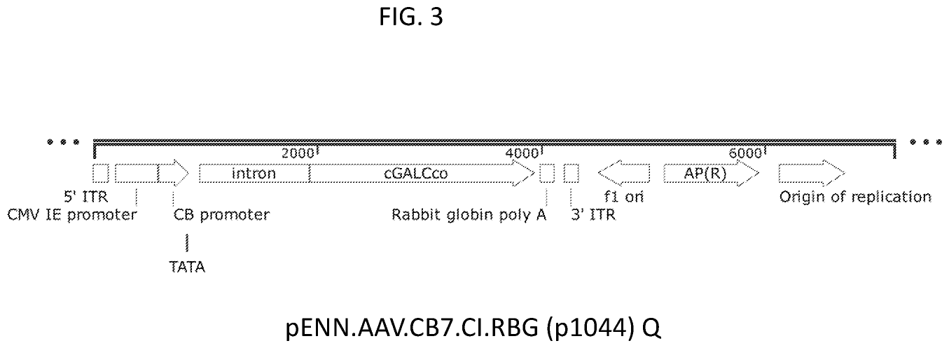

The aim of this study was to assess the scalability of our therapeutic approach by evaluating the efficacy of an AAV vector similar to rAAVhu68.hGALC in a large animal disease model. To accomplish this, we utilized a naturally occurring canine model of Krabbe disease, which was administered via the intended clinical route (ICM) a vector similar to rAAVhu68.hGALC that encodes an engineered canine version of GALC (AAVhu68.CB7.CI.cGALCco.rBG) (FIG. 3). The canine version of GALC was selected as the transgene in order to limit the risk of an exaggerated immune response to a foreign transgene, but the other elements of the vector are equivalent to rAAVhu68.hGALC (including the ubiquitous CB7 promoter and AAVhu68 capsid).

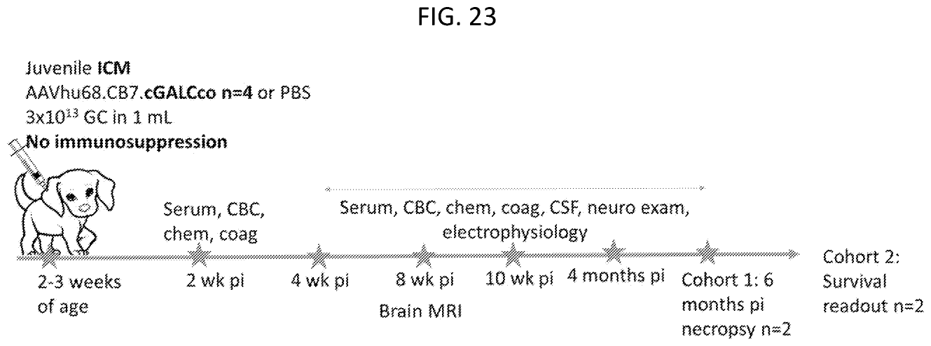

A study design is provided in FIG. 23. Dogs (N=7 total) were injected ICM at the age of 2-3 weeks with either the GALC-expressing AAV vector at a dose of 3.00×10′³ GC (N=4 affected dogs) or vehicle (artificial CSF; N=2 affected dogs; N=1 healthy wild type littermate). The age of the animals was selected to ensure that the dogs were treated as early as possible prior to the onset of behavioral symptoms because rAAVhu68.hGALC was found to be more efficacious in presymptomatic Twitcher mice compared to symptomatic mice treated at later time points (see Example 2).

Group Designations

| Group designation | 1 | 2 | 3 |
|---|---|---|---|
| Number of animals | 1 | 3 | 1 |
| per group | | | |
| Sex | M or F | M or F | M or F |
| Treatment | artificial CSF | AAVhu68.CB7.CI.cGALCco.rBG | artificial CSF |
| ROA | ICM | ICM | ICM |
| Necropsy day | Day 70 or | One euthanized at same | Day 180 |
| | Humane | time point as Goup1, and | |
| | endpoints | two euthanized at Day 180 | |

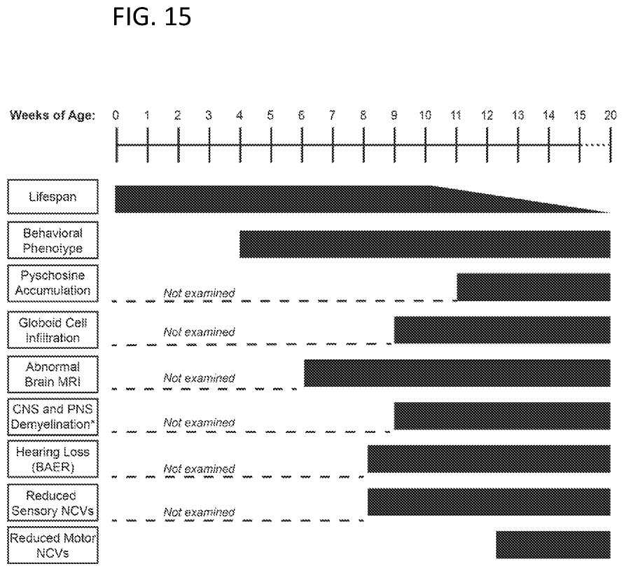

After dosing, animals were monitored daily (cage-side observations), weighed weekly, and videotaped biweekly. They also received a brain MRI and, periodically, a complete physical exam, neurological exam, nerve conduction recordings, and BAER recording. The purpose of these examinations was to evaluate the integrity of the CNS and PNS. Efficacy readouts included brain myelination (assessed by MRI 8 weeks post injection, BAER, and histology at the terminal endpoint), peripheral nerve myelination (assessed by NCV and histology at the terminal endpoint), neurological examination, and physical examination (body weight, gait, reflexes, proprioception, videotaping of dogs playing in an open area biweekly). In addition, pharmacology, safety, and vector biodistribution were assessed because the vector injected was comparable to rAAVhu68.hGALC and the intended clinical ROA (ICM administration) was used. Nerve conduction assessments measured the integrity of nerves as an indirect measurement of myelin integrity. BAER recording is similar to NCV, except it tests conduction and myelin integrity in the auditory pathways of the CNS (i.e., the brainstem). Safety readouts include periodical cell blood counts (CBC), serum chemistry, and coagulation on Day 0 before injection, and Day 14, 28, 56, 70, 120, and 180 (each time ±2 days). CSF was also processed for lipidomic biomarker analysis and cell counts when volume permitted to investigate disease correction (lipidomics, psychosine concentration) and WBC counts (pleocytosis) as a safety readout. On Day 56, dogs were examined by MRI (T1 and T2-weighted) to observe myelination in the CNS. Enzymatic activity of GALC was assessed in both CSF and serum at baseline at the time points indicated below to measure the quantity of active therapeutic enzyme secreted in the CNS and PNS. The study days for each assay were selected in order to minimize the sedation of the animals while collecting complete data at the appropriate time points that correspond to the known Krabbe disease progression in dogs (FIG. 15).

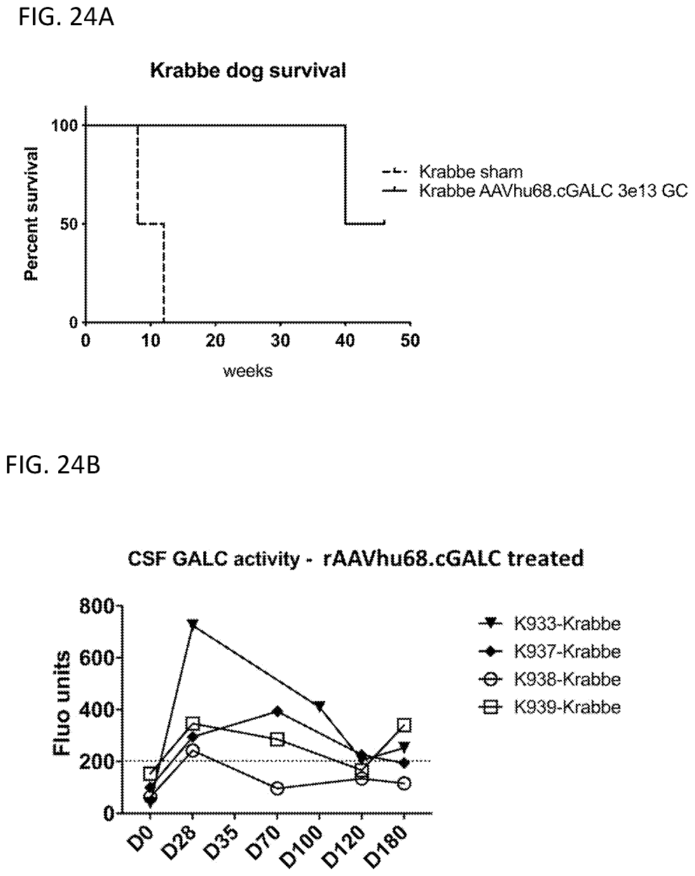

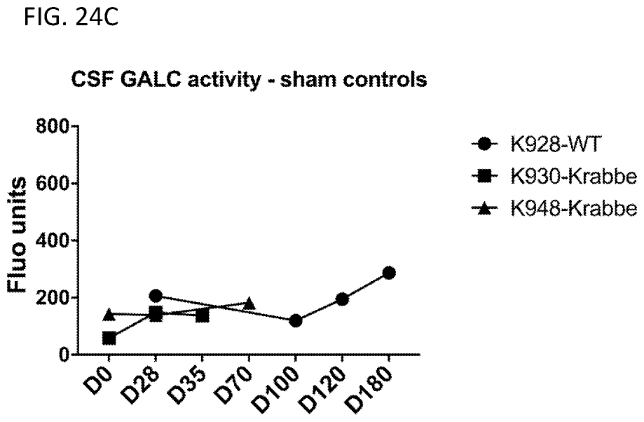

Blood and CSF were collected for safety and biomarker analysis. A comprehensive list of tissues were sampled for histopathology to determine whether the administration of rAAVhu68.hGALC reduces demyelination and neuroinflammation. Transduction and expression of rAAVhu68.hGALC were assessed by biodistribution. GALC enzyme activity readouts (FIG. 24B and FIG. 24C) indicated that all treated Krabbe dogs had rapid enzyme secretion into the CSF.

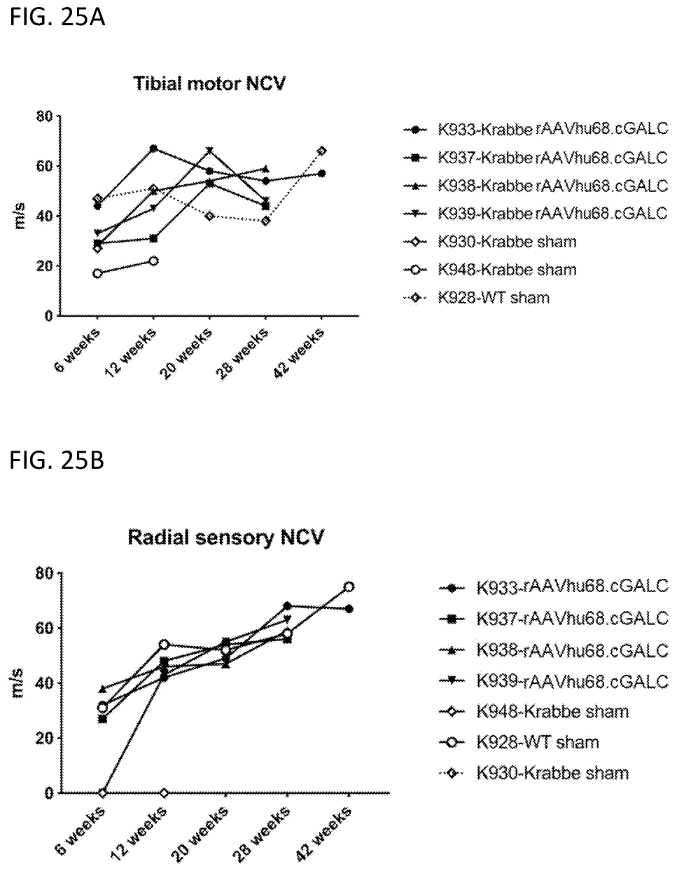

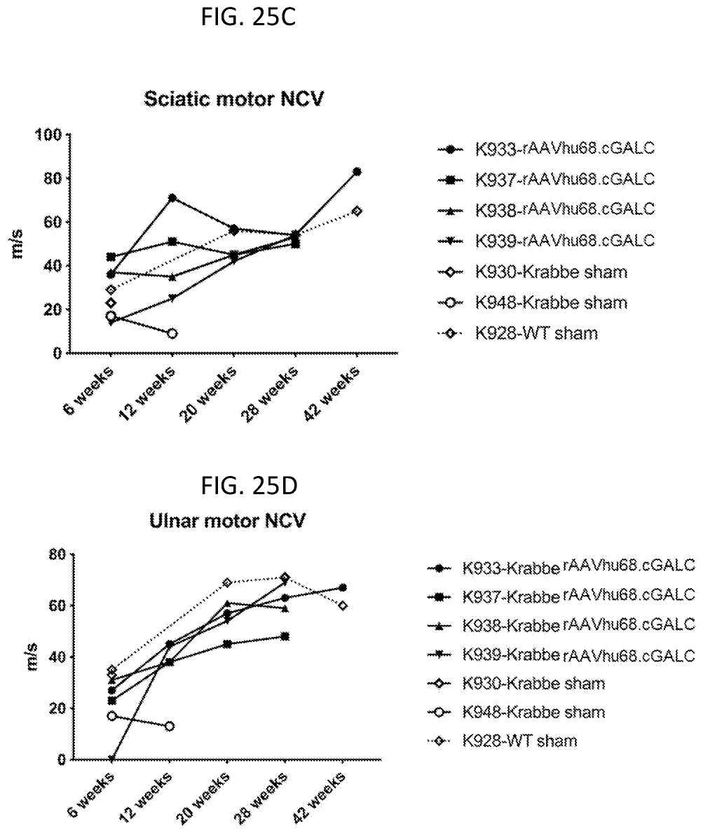

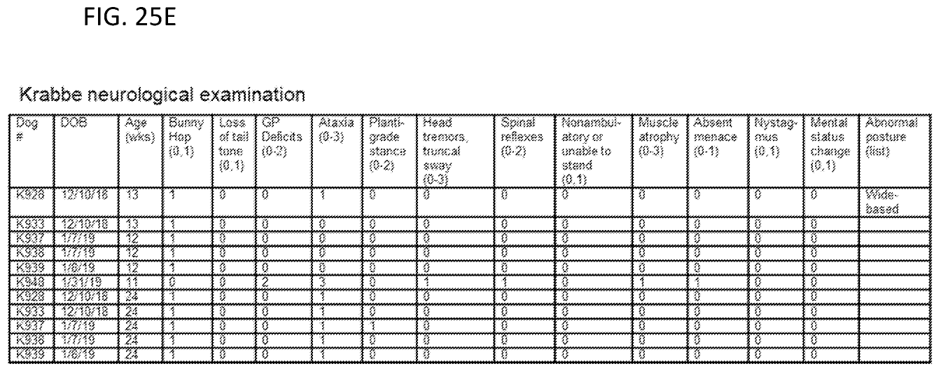

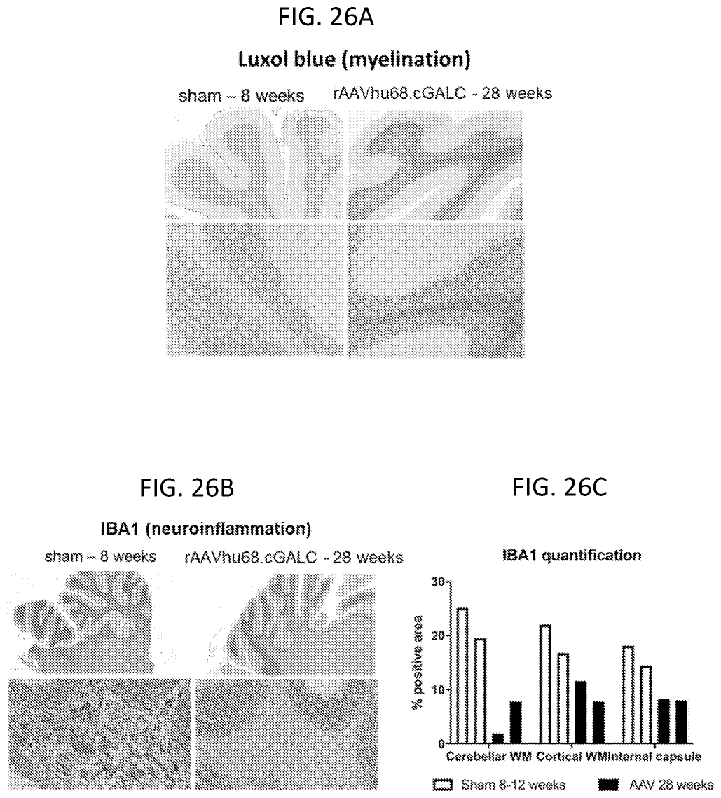

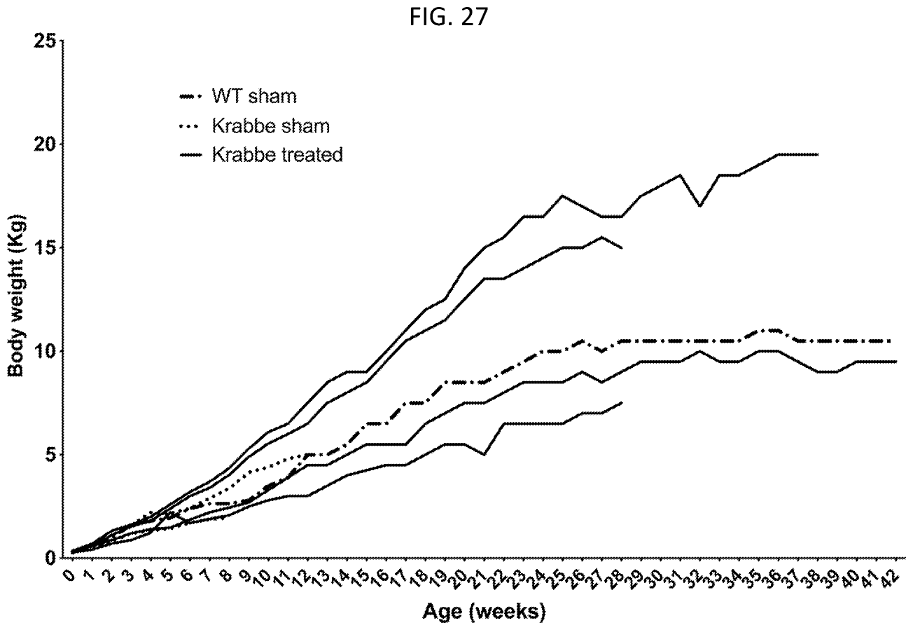

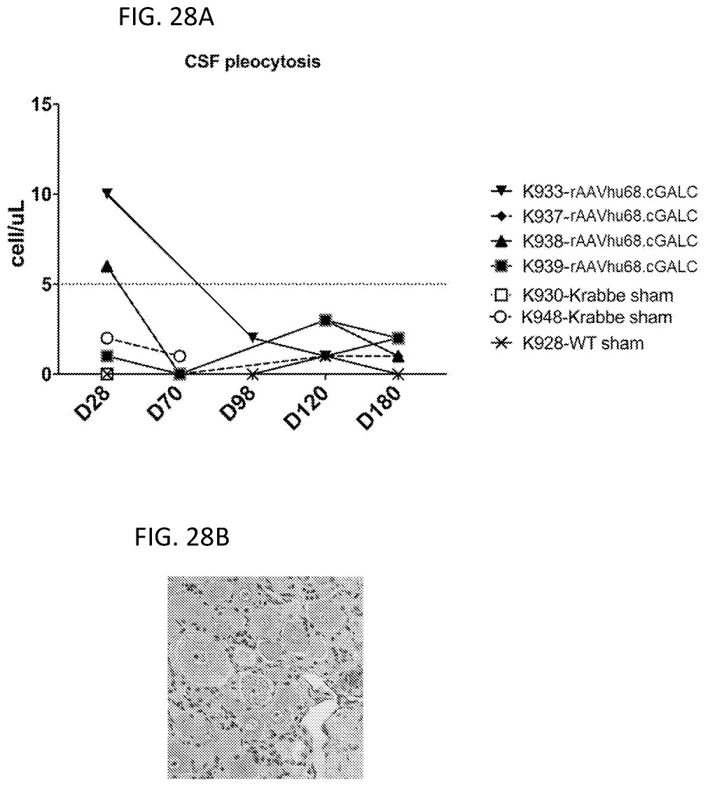

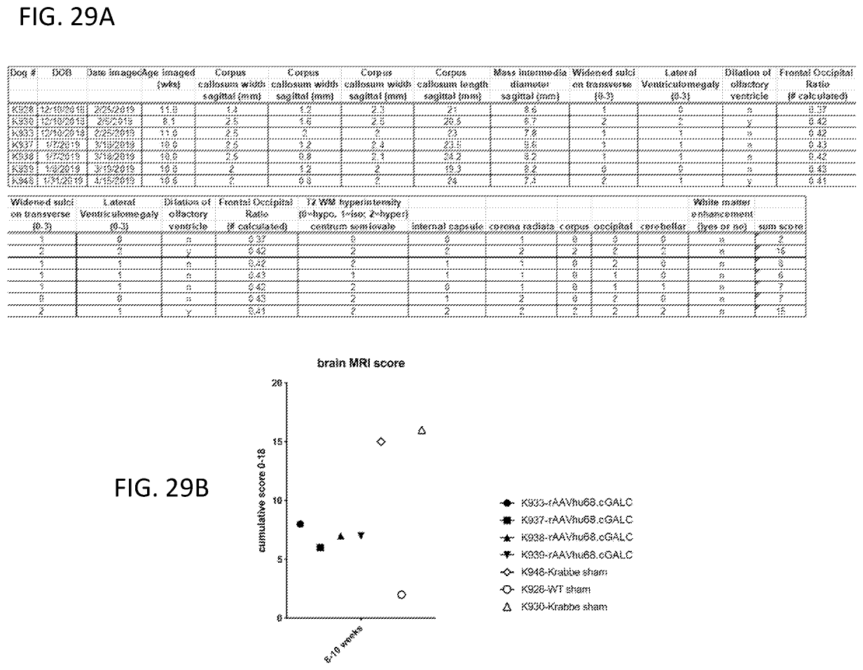

In presymptomatic Krabbe dogs, a single cisterna magna injection of rAAVhu68.cGALC at 3.00×10¹³ GC provided phenotypic correction (FIG. 25E), survival increase (FIG. 24A), nerve conduction normalization (FIG. 25A—FIG. 25D), normal bloodwork, and improved brain MRI (FIG. 29A and FIG. 29B), demonstrating the scalability of the approach. Brain histology showed improved myelination (FIG. 26A) and decreased neuroinflammation (IBA1 staining in cerebellum white matter) (FIG. 26B) in rAAVhu68.cGALC Krabbe dogs relative to controls. None of the dogs initially dosed in this study with either the GALC-expressing vector or vehicle have had adverse events. Krabbe dogs administered rAAVhu68.cGALC exhibited normal growth (FIG. 27) and there was no toxicity observed at six months based on an absence of CSF pleocytosis (FIG. 28A) and histopathological lesions (FIG. 28B). Vehicle-treated dog had to be euthanized at 8 and 12 weeks due to Krabbe disease progression, including hindlimb severe peresis, urinary incontinence, head tremors, and ataxia precluding ambulation. At greater than 45 weeks post injection, all vector-treated dogs appeared bright, alert, and symptom-free, and all had passed the maximal life expectancy of Krabbe dogs.

The study schedule and endpoints are presented below in Table 6.

TABLE 6

| | | Group | Group | Group |
|---|---|---|---|---|
| Study Day | Sample/Procedure | 1 | 2 | 3 |
| PND 0-7 | Genotyping | 1 | 3 | 1 |
| PND 14-21 | ICM vehicle | 1 | — | 1 |
| Study Day 0 | ICM AAVHu68.CB7.cGALC | — | 3 | — |
| | CSF (200 μl for enzymatic activity and | 1 | 3 | 1 |
| | 200 ul for lipids) | | | |
| | Serum up to 200 μL for baseline enzyme | 1 | 3 | 1 |
| | and immunol | | | |
| | CBC/chemistry/coagulation (prioritize | 1 | 3 | 1 |
| | depending on blood volume: 1 = | | | |
| | chemistry, 2 = CBC, 3 = coagulation) | | | |
| Study Day 0 -end | Daily cage side observation | 1 | 3 | 1 |
| of study | Weekly body weight | 1 | 3 | 1 |
| Day 14 | CSF (200 μl for enzymatic activity, 300 to | 1 | 3 | 1 |
| (±1 day) | 500 μL for cell counts and cytology) | | | |
| | Serum up to 300 μL for enzyme and | 1 | 3 | 1 |
| | immunol | | | |
| | CBC/chemistry/coagulation (prioritize | 1 | 3 | 1 |
| | depending on blood volume: 1 = | | | |
| | chemistry, 2 = CBC, 3 = coagulation) | | | |
| Day 28 | CSF (200 uL enzyme activity, 200 μL | 1 | 3 | 1 |
| (±2 days) | lipids, cell count, 300 to 500 μL for cell | | | |
| | counts and cytology) | | | |
| | Serum up to 400 μL for enzyme and | 1 | 3 | 1 |
| | immunol | | | |
| | CBC/chemistry/coagulation (prioritize | 1 | 3 | 1 |
| | depending on blood volume: 1 = | | | |
| | chemistry, 2 = CBC, 3 = coagulation) | | | |
| | Physical and neurological exam | 1 | 3 | 1 |
| | NCV | 1 | 3 | 1 |
| Day 56 | MRI 1.5 tesla brain with T1 and T2- | 1 | 3 | 1 |
| (±2 days) | weighted | | | |
| Day 70 (±3 days | CSF (200 μL enzyme activity, 200 μL | 1 | 3 | 1 |
| OR humane | lipids, cell count, 300 to 500 μL for cell | | | |
| endpoint Group 1 | counts and cytology) | | | |
| if needed before | Serum up to 400 μL for enzyme and | 1 | 3 | 1 |
| D 70 | immunol | | | |
| | CBC/chemistry/coagulation | 1 | 3 | 1 |
| | Physical and neurological exam | 1 | 3 | 1 |
| | NCV | 1 | 3 | 1 |
| | BAER | 1 | 3 | 1 |
| Day 70 (±3 days | Necropsy | 1 | 1 | — |
| OR humane | | | | |
| endpoint Group 1 | | | | |
| if needed before | | | | |
| D 70 | | | | |
| Day 120 | CSF (200 μL enzyme activity, 200 μL | — | 2 | 1 |
| (±3 days) | lipids, cell count, 300 to 500 μL for cell | | | |
| | counts and cytology) | | | |
| | Serum up to 400 μL for enzyme and | — | 2 | 1 |
| | immunol | | | |
| | CBC/chemistry/coagulation | — | 2 | 1 |
| | Physical and neurological exam | — | 2 | 1 |
| | NCV | — | 2 | 1 |
| Day 180 | CSF (200 μL enzyme activity, 200 μL | — | 2 | 1 |
| (±3 days) | lipids, cell count, 300 to 500 μL for cell | | | |
| | counts and cytology) | | | |
| | Serum up to 400 μL for enzyme and | — | 2 | 1 |
| | immunol | | | |
| | CBC/chemistry/coagulation | — | 2 | 1 |
| | | — | | |
| | Physical and neurological exam | — | 2 | 1 |
| | NCV | — | 2 | 1 |
| | BAER | — | 2 | 1 |
| | Necropsy | — | 2 | 1 |

### Example 5—Toxicology Study in Nonhuman Primates

A toxicology study is conducted using the same rAAVhu68.hGALC vector lot as that used in the mouse MED study and is conducted in NHPs because they better replicate the size and CNS anatomy of humans and can be treated using the clinical ROA (ICM). It is expected that the similarity in size, anatomy, and ROA results in representative vector distribution and transduction profiles, which allows for more accurate assessment of toxicity than is possible in mice or dogs. In addition, more rigorous neurological assessments can be performed in NHPs than in rodent or canine models, allowing for more sensitive detection of CNS toxicity.

ICM vector administration results in immediate vector distribution within the CSF compartment. Doses are scaled by brain mass, which provides an approximation of the size of the CSF compartment. Dose conversions are based on a brain mass of 0.15 g for a newborn mouse (Gu Z., et al. (2012) PLoS One. 7(7):e41542.), 0.4 g for a juvenile-adult mouse (Gu Z., et al. (2012) PLoS One. 7(7):e41542.), 90 g for a juvenile and adult rhesus macaque (Herndon J. G., et al. (1998) Neurobiol Aging. 19(3):267-72), 60 g for a dog, 800 g for 4-12-month-old infants, and 1300 g for adult humans (Dekaban A. S. (1978) Ann Neurol. 4(4):345-56). Doses for the NHP toxicology study, the murine MED study, and the equivalent human doses are shown in Table 7.

TABLE 7
Vector doses for the murine MED study, NHP toxicology
study, and equivalent dog and human doses:

| Dose | Juvenile | Juvenile NHP | | |
|---|---|---|---|---|
| (GC/g | Mouse MED | toxicology | | |
| brain | study | study | Dog | Human |
| mass) | (GC) | (GC) | (GC) | (GC) |
| 5.00 × 10¹¹ | 2.00 × 10¹¹ | 4.50 × 10¹³ | 3.00 × 10¹³ | 4.00 × 10¹⁴ |
| 1.70 × 10¹¹ | 6.80 × 10¹⁰ | 1.50 × 10¹³ | — | 1.40 × 10¹⁴ |
| 5.00 × 10¹⁰ | 2.00 × 10¹⁰ | 4.50 × 10¹² | — | 4.00 × 10¹³ |
| 1.70 × 10¹⁰ | 6.80 × 10⁹ | — | — | 1.40 × 10¹³ |

Abbreviations: GC, genome copies; MED, minimum effective dose; NHP, nonhuman primate.

Juvenile rhesus macaques are selected depending on disease target in order to be similar anatomically to the proposed Phase ½ study population. The doses for the NHP toxicology study reflect the doses that are used in a Phase ½ clinical study, and are selected with consideration of: 1) results from the pharmacology studies and 2) translation of dose from the pharmacology studies to NHP and human, with consideration of the maximal feasible dose.

Accordingly, a 180-day GLP-compliant safety study is conducted in adult rhesus macaques to investigate the toxicology of rAAVhu68.CB7.CI.cGALCco.rBG (rAAVhu68.hGALC) following ICM administration. The 180-day evaluation period was selected because this allows sufficient time for a secreted transgene product to reach stable plateau levels following ICM AAV administration. The study design is outlined in Table 8 and Table 9. Juvenile Rhesus macaques (approximately 1.5 years of age) receive either 4.50×10¹² GC total or 1.50×10¹³ GC total (or vehicle). Dose levels are selected to be equivalent to those are evaluated in the MED study when scaled by brain mass (assuming 0.4 g for the juvenile-adult mouse, 90 g for the rhesus monkey. The high dose is equivalent to the dose evaluated in the Krabbe dog model (assuming a brain weight of 60 g). Baseline neurologic examinations, clinical pathology (cell counts with differentials, clinical chemistries, and a coagulation panel), CSF chemistry, and CSF cytology are performed. Following vector (or vehicle) administration, the animals are monitored daily for signs of distress and abnormal behavior.

Blood and CSF clinical pathology assessments and neurologic examinations are performed on a weekly basis for 30 days following vector or vehicle administration, and every 30 days thereafter. At baseline and at each 30-day time point thereafter, anti-AAVhu68 NAbs and cytotoxic T lymphocyte (CTL) responses to AAVhu68 and the GALC product are assessed by an interferon gamma (IFN-γ) enzyme-linked immunospot (ELISpot) assay.

At either 90 or 180 days following rAAVhu68.hGALC or vehicle administration, animals are euthanized, and tissues are harvested for comprehensive microscopic histopathological examination. The histopathological examination focuses on CNS tissues (brain, spinal cord, and dorsal root ganglia) and the liver because these are the most heavily transduced tissues following ICM administration of rAAVhu68 vectors. In addition, lymphocytes are harvested from the systemic circulation (PBMC), spleen, and CNS-draining lymph nodes to evaluate the presence of T cells reactive to both the capsid and transgene product in these organs at the time of necropsy. Tissues are harvested and archived in case any finding warrants further analysis of vector biodistribution.

TABLE 8
Rhesus Macaque GLP Toxicology Study (Group Designations)

| Group Designation | 1 | 2 | 3 | 4 | 5 | 6 | 7 | 8 |
|---|---|---|---|---|---|---|---|---|
| rAAVhu68.hGALC/ | ITFFB | rAAV | rAAV | rAAV | ITFFB | rAAV | rAAV | rAAV |
| ITFFB | | | | | | | | |
| Dose | N/A | 4.5.0 × | 1.5 × | 4.5.0 × | NA | 4.5.0 × | 1.5 × | 4.5.0 × |
| (GC) | | 10¹² | 10¹³ | 10¹³ | | 10¹² | 10¹³ | 10¹³ |
| Number of | 1 | 3 | 3 | 3 | 1 | 3 | 3 | 3 |
| Macaques | | | | | | | | |
| Sex | Either | Both | Both | Both | Either | Both | Both | Both |
| ROA | ICM | ICM | ICM | ICM | ICM | ICM | ICM | ICM |
| Necropsy | 90 ± 4 | 90 ± 4 | 90 ± 4 | 90 ± 4 | 180 ± 5 | 180 ± 5 | 180 ± 5 | 180 ± 5 |
| Day | | | | | | | | |

Abbreviations: F, female; GLP, good laboratory practice; ICM, intra-cistema magna; M, male; NA, not applicable; ROA, route of administration.

TABLE 9
Rhesus Macaque Study Schedule

| Study Time | Sample/ | Group | Group | Group | Group | Group | Group | Group | Group |
|---|---|---|---|---|---|---|---|---|---|
| point | Procedure | 1^{a} | 2^{a} | 3^{a} | 4^{a} | 5^{a} | 6^{a} | 7^{a} | 8^{a} |
| Baseline - | Body Weight, | 1 | 3 | 3 | 3 | 1 | 3 | 3 | 3 |
| up to 28 | Temperature, | | | | | | | | |
| days prior to | Respiratory Rate, | | | | | | | | |
| dosing | Heart Rate | | | | | | | | |
| | Neurological | 1 | 3 | 3 | 3 | 1 | 3 | 3 | 3 |
| | Monitoring | | | | | | | | |
| | Biomarker - Blood | 1 | 3 | 3 | 3 | 1 | 3 | 3 | 3 |
| | (baseline) | | | | | | | | |
| | Clin path | 1 | 3 | 3 | 3 | 1 | 3 | 3 | 3 |
| | (baseline)^{b} | | | | | | | | |
| | NAb (baseline) | 1 | 3 | 3 | 3 | 1 | 3 | 3 | 3 |
| | Immunology - | 1 | 3 | 3 | 3 | 1 | 3 | 3 | 3 |
| | ELISPOT (baseline) | | | | | | | | |
| | (PBMC) | | | | | | | | |
| | Vector Excretion | 1 | 3 | 3 | 3 | 1 | 3 | 3 | 3 |
| | (urine, feces) | | | | | | | | |
| | Nerve conduction | 1 | 3 | 3 | 3 | 1 | 3 | 3 | 3 |
| | velocity testing | | | | | | | | |
| Study Day 0 | Body Weight, | 1 | 3 | 3 | 3 | 1 | 3 | 3 | 3 |
| | Temperature, | | | | | | | | |
| | Respiratory Rate, | | | | | | | | |
| | Heart Rate | | | | | | | | |
| | Clin path^{b} | 1 | 3 | 3 | 3 | 1 | 3 | 3 | 3 |
| | CSF^{c} | 1 | 3 | 3 | 3 | 1 | 3 | 3 | 3 |
| | Vector PK CSF | 1 | 3 | 3 | 3 | 1 | 3 | 3 | 3 |
| | Vector PK Blood | 1 | 3 | 3 | 3 | 1 | 3 | 3 | 3 |
| | ITFFB ICM | 1 | — | — | — | — | — | — | — |
| | AAV.hGALC, | — | 3 | — | — | — | 3 | — | — |
| | 4.5.0 × 10¹² GC, ICM | | | | | | | | |
| | AAV.hGALC, | — | — | 3 | — | — | — | 3 | — |
| | 1.5 × 10¹³ GC, ICM | | | | | | | | |
| | AAV.hGALC, | — | — | — | 3 | — | — | — | 3 |
| | 4.5 × 10¹³ GC, ICM | | | | | | | | |
| Study Day 5 | Vector Excretion | 1 | 3 | 3 | 3 | 1 | 3 | 3 | 3 |
| (±2 days) | (urine, feces) | | | | | | | | |
| Study Day 7 | Body Weight, | 1 | 3 | 3 | 3 | 1 | 3 | 3 | 3 |
| (±1 days) | Temperature, | | | | | | | | |
| | Respiratory Rate, | | | | | | | | |
| | Heart Rate | | | | | | | | |
| | Clin path^{b} | 1 | 3 | 3 | 3 | 1 | 3 | 3 | 3 |
| | CSF^{c} | 1 | 3 | 3 | 3 | 1 | 3 | 3 | 3 |
| | Vector PK CSF | 1 | 3 | 3 | 3 | 1 | 3 | 3 | 3 |
| | Vector PK blood | 1 | 3 | 3 | 3 | 1 | 3 | 3 | 3 |
| Study Day 14 | Body Weight, | 1 | 3 | 3 | 3 | 1 | 3 | 3 | 3 |
| (±2 days) | Temperature, | | | | | | | | |
| | Respiratory Rate, | | | | | | | | |
| | Heart Rate | | | | | | | | |
| | Neurological | 1 | 3 | 3 | 3 | 1 | 3 | 3 | 3 |
| | Monitoring | | | | | | | | |
| | Biomarker - Blood | 1 | 3 | 3 | 3 | 1 | 3 | 3 | 3 |
| | Clin path^{b} | 1 | 3 | 3 | 3 | 1 | 3 | 3 | 3 |
| | CSF^{c} | 1 | 3 | 3 | 3 | 1 | 3 | 3 | 3 |
| | Vector PK CSF | 1 | 3 | 3 | 3 | 1 | 3 | 3 | 3 |
| | Vector PK Blood | 1 | 3 | 3 | 3 | 1 | 3 | 3 | 3 |
| Study Day 28 | Body Weight, | 1 | 3 | 3 | 3 | 1 | 3 | 3 | 3 |
| (±3 days) | Temperature, | | | | | | | | |
| | Respiratory Rate, | | | | | | | | |
| | Heart Rate | | | | | | | | |
| | Neurological | 1 | 3 | 3 | 3 | 1 | 3 | 3 | 3 |
| | Monitoring | | | | | | | | |
| | Biomarker - Blood | 1 | 3 | 3 | 3 | 1 | 3 | 3 | 3 |
| | Clin path^{b} | 1 | 3 | 3 | 3 | 1 | 3 | 3 | 3 |
| | CSF^{c} | 1 | 3 | 3 | 3 | 1 | 3 | 3 | 3 |
| | Vector PK Blood | 1 | 3 | 3 | 3 | 1 | 3 | 3 | 3 |
| | Vector PK CSF | 1 | 3 | 3 | 3 | 1 | 3 | 3 | 3 |
| | Immunology - | 1 | 3 | 3 | 3 | 1 | 3 | 3 | 3 |
| | ELISPOT (PBMC) | | | | | | | | |
| | NAb | 1 | 3 | 3 | 3 | 1 | 3 | 3 | 3 |
| | Vector Excretion | 1 | 3 | 3 | 3 | 1 | 3 | 3 | 3 |
| | (urine, feces) | | | | | | | | |
| | Nerve conduction | 1 | 3 | 3 | 3 | 1 | 3 | 3 | 3 |
| | velocity testing | | | | | | | | |
| Study Day 60 | Body Weight, | 1 | 3 | 3 | 3 | 1 | 3 | 3 | 3 |
| (±3 days) | Temperature, | | | | | | | | |
| | Respiratory Rate, | | | | | | | | |
| | Heart Rate | | | | | | | | |
| | Neurological | 1 | 3 | 3 | 3 | 1 | 3 | 3 | 3 |
| | Monitoring | | | | | | | | |
| | Biomarker - Blood | 1 | 3 | 3 | 3 | 1 | 3 | 3 | 3 |
| | Clin path^{b} | 1 | 3 | 3 | 3 | 1 | 3 | 3 | 3 |
| | CSF^{c} | 1 | 3 | 3 | 3 | 1 | 3 | 3 | 3 |
| | Vector PK CSF | 1 | 3 | 3 | 3 | 1 | 3 | 3 | 3 |
| | Vector PK Blood | 1 | 3 | 3 | 3 | 1 | 3 | 3 | 3 |
| | NAb | 1 | 3 | 3 | 3 | 1 | 3 | 3 | 3 |
| | Immunology - | 1 | 3 | 3 | 3 | 1 | 3 | 3 | 3 |
| | ELISPOT (PBMC) | | | | | | | | |
| | Vector Excretion | 1 | 3 | 3 | 3 | 1 | 3 | 3 | 3 |
| | (urine, feces) | | | | | | | | |
| | Nerve conduction | 1 | 3 | 3 | 3 | 1 | 3 | 3 | 3 |
| | velocity testing | | | | | | | | |
| Study Day 90 | Body Weight, | 1 | 3 | 3 | 3 | 1 | 3 | 3 | 3 |
| (±4 days) | Temperature, | | | | | | | | |
| | Respiratory Rate, | | | | | | | | |
| | Heart Rate | | | | | | | | |
| | Neurological | 1 | 3 | 3 | 3 | 1 | 3 | 3 | 3 |
| | monitoring | | | | | | | | |
| | Biomarker - Blood | 1 | 3 | 3 | 3 | 1 | 3 | 3 | 3 |
| | Clin path^{b} | 1 | 3 | 3 | 3 | 1 | 3 | 3 | 3 |
| | CSF^{c} | 1 | 3 | 3 | 3 | 1 | 3 | 3 | 3 |
| | Vector PK CSF | 1 | 3 | 3 | 3 | 1 | 3 | 3 | 3 |
| | Vector PK Blood | 1 | 3 | 3 | 3 | 1 | 3 | 3 | 3 |
| | NAb | 1 | 3 | 3 | 3 | 1 | 3 | 3 | 3 |
| | Immunology - | 1 | 3 | 3 | 3 | 1 | 3 | 3 | 3 |
| | ELISPOT (PBMC) | | | | | | | | |
| | Vector Excretion | 1 | 3 | 3 | 3 | 1 | 3 | 3 | 3 |
| | (urine, feces) | | | | | | | | |
| | Nerve conduction | 1 | 3 | 3 | 3 | 1 | 3 | 3 | 3 |
| | velocity testing | | | | | | | | |
| | Necropsy | 1 | 3 | 3 | 3 | — | — | — | — |
| Study Day 120 | Body Weight, | — | — | — | — | 1 | 3 | 3 | 3 |
| (±4 days) | Temperature, | | | | | | | | |
| | Respiratory Rate, | | | | | | | | |
| | Heart Rate | | | | | | | | |
| | Neurological | — | — | — | — | 1 | 3 | 3 | 3 |
| | Monitoring | | | | | | | | |
| | Biomarker - Blood | — | — | — | — | 1 | 3 | 3 | 3 |
| | Clin path^{b} | — | — | — | — | 1 | 3 | 3 | 3 |
| | CSF^{c} | — | — | — | — | 1 | 3 | 3 | 3 |
| | NAb | — | — | — | — | 1 | 3 | 3 | 3 |
| | Immunology - | — | — | — | — | 1 | 3 | 3 | 3 |
| | ELISPOT (PBMC) | | | | | | | | |
| | Vector Excretion | — | — | — | — | 1 | 3 | 3 | 3 |
| | (urine, feces) | | | | | | | | |
| | Nerve conduction | — | — | — | — | 1 | 3 | 3 | 3 |
| | velocity testing | | | | | | | | |
| Study Day 150 | Body Weight, | — | — | — | — | 1 | 3 | 3 | 3 |
| (±4 days) | Temperature, | | | | | | | | |
| | Respiratory Rate, | | | | | | | | |
| | Heart Rate | | | | | | | | |
| | Neurological | — | — | — | — | 1 | 3 | 3 | 3 |
| | Monitoring | | | | | | | | |
| | Biomarker - Blood | — | — | — | — | 1 | 3 | 3 | 3 |
| | Clin path^{b} | — | — | — | — | 1 | 3 | 3 | 3 |
| | CSF^{c} | — | — | — | — | 1 | 3 | 3 | 3 |
| | NAb | — | — | — | — | 1 | 3 | 3 | 3 |
| | Immunology - | — | — | — | — | 1 | 3 | 3 | 3 |
| | ELISPOT (PBMC) | | | | | | | | |
| | Vector Excretion | — | — | — | — | 1 | 3 | 3 | 3 |
| | (urine, feces) | | | | | | | | |
| | Nerve conduction | — | — | — | — | 1 | 3 | 3 | 3 |
| | velocity testing | | | | | | | | |
| Study Day 180 | Body Weight, | — | — | — | — | 1 | 3 | 3 | 3 |
| (±5 days) | Temperature, | | | | | | | | |
| | Respiratory Rate, | | | | | | | | |
| | Heart Rate | | | | | | | | |
| | Neurological | — | — | — | — | 1 | 3 | 3 | 3 |
| | Monitoring | | | | | | | | |
| | Biomarker - Blood | — | — | — | — | 1 | 3 | 3 | 3 |
| | Clin path^{b} | — | — | — | — | 1 | 3 | 3 | 3 |
| | CSF^{c} | — | — | — | — | 1 | 3 | 3 | 3 |
| | NAb | — | — | — | — | 1 | 3 | 3 | 3 |
| | Vector PK CSF | — | — | — | — | 1 | 3 | 3 | 3 |
| | Vector PK Blood | — | — | — | — | 1 | 3 | 3 | 3 |
| | Immunology - | — | — | — | — | 1 | 3 | 3 | 3 |
| | ELISPOT (PBMC) | | | | | | | | |
| | Vector Excretion | — | — | — | — | 1 | 3 | 3 | 3 |
| | (urine, feces) | | | | | | | | |
| | Nerve conduction | — | — | — | — | 1 | 3 | 3 | 3 |
| | velocity testing | | | | | | | | |
| | Necropsy | — | — | — | — | 1 | 3 | 3 | 3 |

^{a}Number of animals assessed.
^{b}Includes complete blood counts and differentials (hematology), clinical chemistries and coagulation panel
^{c}Includes clinical pathology and biomarker
Abbreviations:
NAb, neutralizing antibodies;
PBMC, peripheral blood mononuclear cells;
CSF, cerebrospinal fluid;
Clin Path, clinical pathology;
PK pharmacokinetic

### Example 6—Treatment of Krabbe Disease with rAAVhu68.hGALC

The FIH trial is a Phase ½ dose escalation study of a single ICM administration of rAAVhu68.hGALC in pediatric patients with the infantile form of Krabbe disease caused by homozygous or compound heterozygous mutations in the GALC gene. This FIH trial enrolls and treats at least 12 subjects who are followed up for 2 years, with continued long-term follow-up (LTFU) for a total of 5 years post-dose in line with the recommended LTFU for adenoviral vectors described in the draft “FDA Guidance for Industry: Long Term Follow-Up after Administration of Human Gene Therapy Products” (July 2018). The primary objectives are to assess the safety and tolerability of rAAVhu68.hGALC. The secondary objectives of this study are to evaluate the impact of rAAVhu68.hGALC on disease-relevant assessments, including survival, age-appropriate neurocognitive measurements, and age-appropriate motor and/or linguistic assessments. These endpoints are selected in consultation with disease experts and clinicians and based on observations on the disease evolution in untreated patients with infantile Krabbe disease.

Optionally, combination therapy of HSCT and AAV gene therapy can be evaluated.

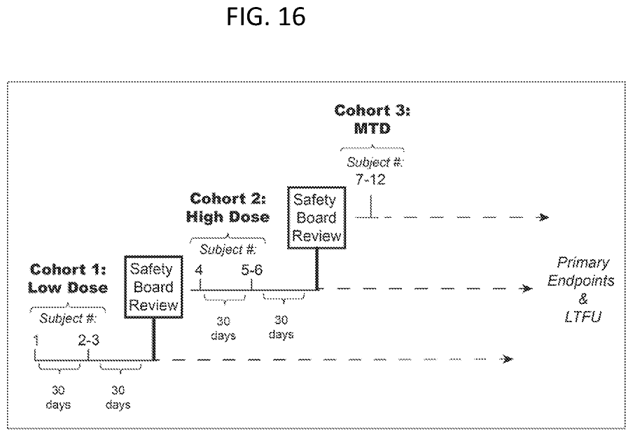

The FIH is an open-label, multi-center, dose escalation study of rAAVhu68.hGALC to evaluate safety, tolerability, and exploratory efficacy endpoints in pediatric subjects with the infantile form of Krabbe disease. The dose-escalation phase assesses the safety and tolerability of a single ICM administration of two dose levels of rAAVhu68.hGALC, with staggered, sequential dosing of subjects. The rAAVhu68.hGALC dose levels are determined based on data from the GLP NHP toxicology study and the murine (MED) study and consist of a low dose (administered to Cohort 1) and a high dose (administered Cohort 2). Both dose levels are anticipated to confer therapeutic benefit, with the understanding that, if tolerated, the higher dose is expected to be advantageous. The sequential evaluation of the low dose followed by the high dose enables the identification of the maximum tolerated dose (MTD) of the doses tested. Finally, an expansion cohort (Cohort 3) receives the MTD of rAAVhu68.hGALC (FIG. 16). Rapid GALC enzyme production following administration of the vector (with 1 week post-treatment) provides an extended therapeutic window.

Since infantile Krabbe Disease is marked by a rapid disease course once symptoms emerge, and given that some neonates present with signs of disease at birth, the proposed study design allows for concurrent enrollment of subjects 30 days after dosing of the first patient in Cohort 1 (low dose) and Cohort 2 (high dose), based on the Investigator's benefit-risk assessment for that subject. The rationale for this is that the risk of missing the treatment window because the patient experienced disease progression would outweigh the potential benefit of prolonged safety follow-up before dosing the next patient. Such a scenario where patients experience substantial disease progression in a matter of weeks was cited as a possible cause of the poor transplant outcomes observed in EIKD patients identified through NBS in New York (Wasserstein M. P., et al. (2016) Genet Med. 18(12):1235-1243), highlighting the need for prompt referral of patients for treatment.

An independent Safety Board conducts a safety review of all accumulated safety data between cohorts and after full enrollment of the second cohort to make a recommendation regarding further conduct of the trial. The Safety Board also conducts a review any time a safety review trigger (SRT) is observed. The 1-month dosing interval between the first and second subject in each cohort allows for evaluation of AEs indicative of acute immune reactions, immunogenicity or other dose-limiting toxicities as well as clinical review of any sensory neuropathy that might present itself consistent with the anticipated time course for development of sensory neuropathology secondary to transduction of DRG, which occurs within 2-4 weeks in non-clinical studies.

Additional subjects are enrolled in an expansion cohort that receives the MTD. Enrollment of these additional subjects does not require a 4-week observation window between subjects (FIG. 16). Optionally, this cohort receives combination treatment with HSCT and rAAVhu68.hGALC.

All treated subjects are followed for 2 years to evaluate the safety profile and to characterize the pharmacodynamic and efficacy properties of rAAVhu68.hGALC. Subjects are followed for an additional 3 years (for a total of 5 years post-dose) during the LTFU period of the study to evaluate long-term clinical outcomes, which is in line with draft “FDA Guidance for Industry: Long Term Follow-Up after Administration of Human Gene Therapy Products” (July 2018).

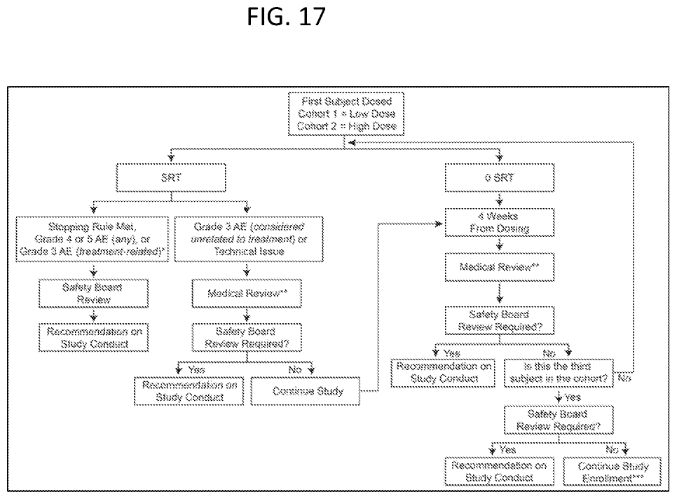

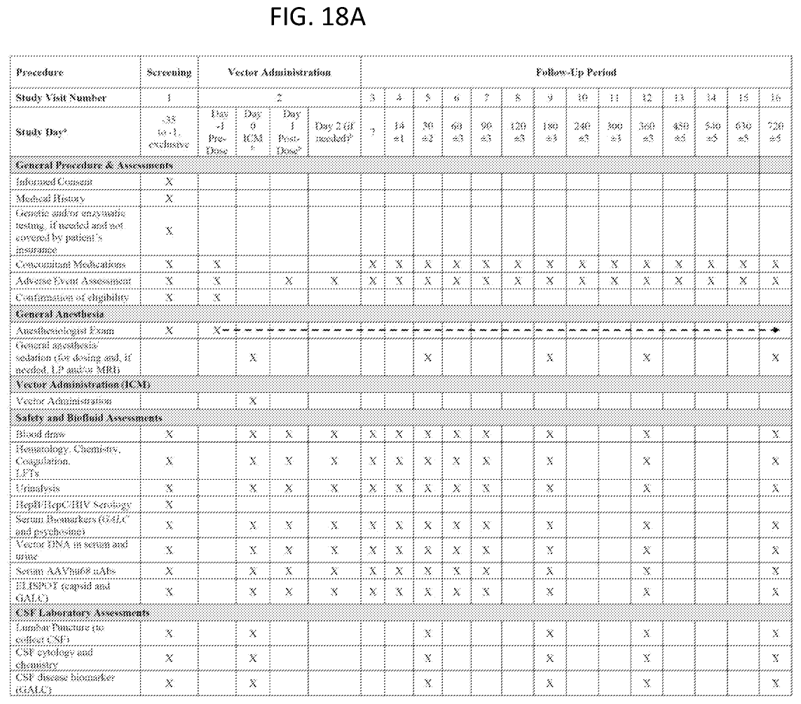

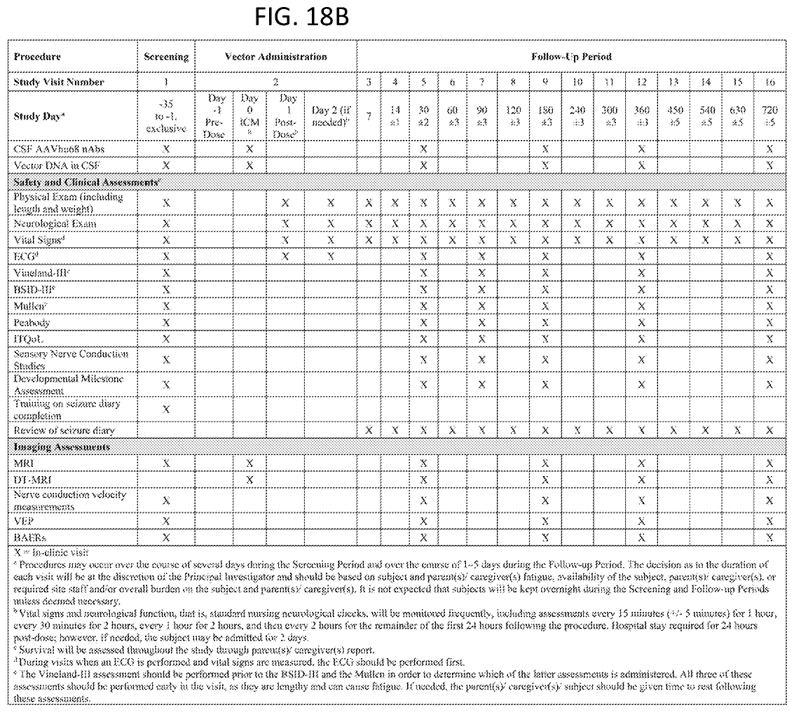

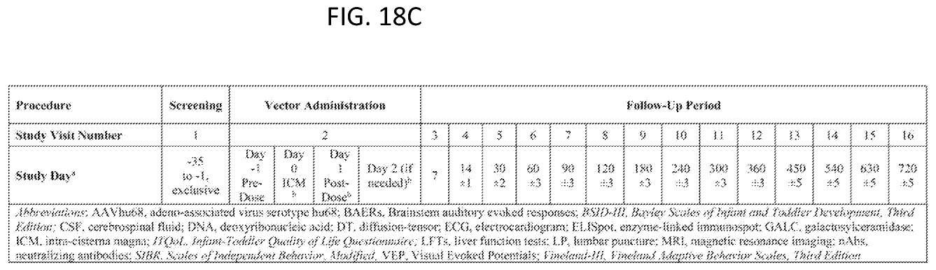

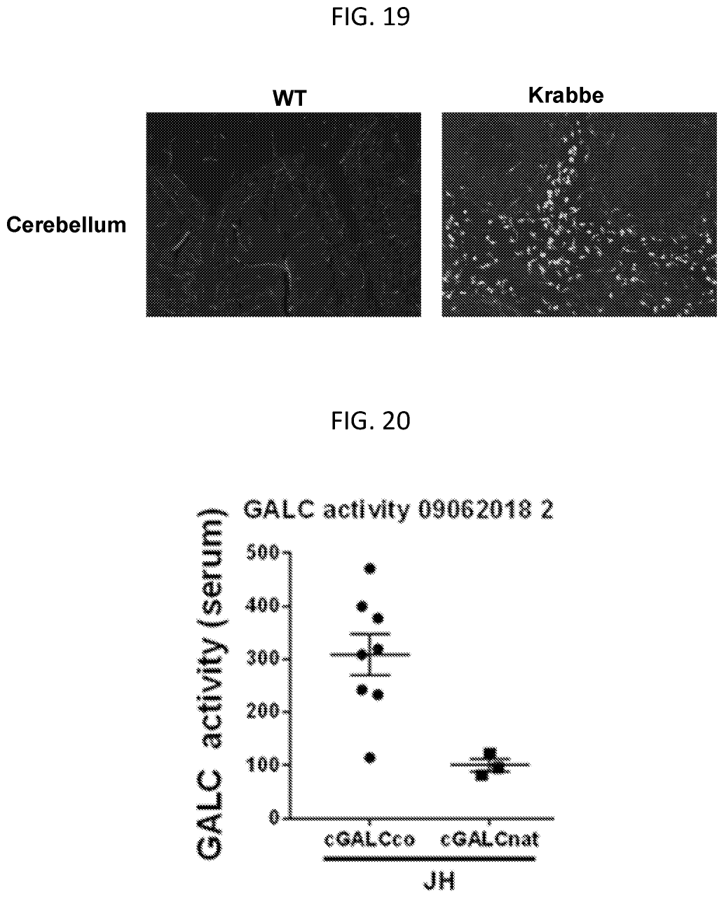

TABLE 10
First-in-Human Clinical Trial Protocol Synopsis

| Protocol(s) Title: | A Phase ½ Open-Label, Multi-Center, Dose Escalation Study to | |
|---|---|---|
| | Assess the Safety and Tolerability of Single Doses of | |
| | rAAVhu68.hGALC Delivered into the Cisterna Magna (ICM) of | |
| | Pediatric Subjects with Infantile Globoid Cell Leukodystrophy | |
| | (Krabbe Disease) | |
| Number of | Up to 12 evaluable subjects | |
| Subjects: | | |
| Objectives: | Primary: | |
| | To assess the safety and tolerability of rAAVhu68.hGALC | |
| | through 24 months following administration of a single ICM | |
| | dose through evaluation of: | |
| | Adverse events (AEs) and serious adverse events (SAEs) | |
| | Vital signs and physical examinations | |
| | Neurological examinations | |
| | Electrocardiograms (ECGs) | |
| | Sensory nerve conduction studies (for evaluation of DRG | |
| | toxicity) | |
| | Laboratory assessments (serum chemistry, hematology, | |
| | coagulation studies, liver function tests (LFTs), urinalysis, | |
| | and CSF chemistry and cytology) | |
| | Immunogenicity of the vector and transgene product | |
| | Secondary (exploratory efficacy): | |
| | To assess the pharmacodynamics and biological activity of | |
| | rAAVhu68.hGALC over 2 years following administration of a | |
| | single ICM dose, based on the following endpoints: | |
| | Levels of GALC in CSF and serum | |
| | To assess the efficacy of rAAVhu68.hGALC through 2 years | |
| | following administration of a single ICM dose as measured by: | |
| | Survival | |
| | Disease progression as assess by age at achievement, age at | |
| | loss and percentage of children maintaining or acquiring | |
| | age appropriate developmental milestones | |
| | Disease progression as assessed by age at achievement, age | |
| | at loss, and percentage of subjects maintaining motor | |
| | milestones (as defined by WHO criteria) and percentage of | |
| | subjects progressing in disease staging. | |
| | Exploratory: | |
| | To further assess the efficacy of rAAVhu68.hGALC through | |
| | 2 years following administration of a single ICM dose, as | |
| | measured by: | |
| | Age-at-onset and frequency of seizures as assessed by a | |
| | seizure dairy | |
| | Clinical outcomes as assessed by the Bayley Scale (BSID- | |
| | III) or the Mullen Scales of early learning (depending on | |
| | age of the subject), Vineland Adaptive Behavior Scales | |
| | (Edition III), Peabody Developmental Motor Scales, Infant | |
| | and Toddler Quality of Life Questionnaire | |
| | To further assess the pharmacodynamic effects of | |
| | rAAVhu68.hGALC through 24 months following | |
| | administration of a single ICM dose, as measured by: | |
| | Central nervous system myelination as measured by MRI | |
| | and DT-MRI | |
| | Nerve conduction velocity (NCV) measurements of the | |
| | deep peroneal, tibial, ulnar, sural, median nerves (to assess | |
| | sensory and motor nerve peripheral neuropathy) | |
| | Visual evoked potentials (VEPs) | |
| | Brainstem auditory evoked responses (BAERs) | |
| | CSF and plasma/serum biomarkers of disease, including | |
| | psychosine and others | |
| Study Design: | Phase ½, FIH, multi-center, open-label, single-arm, dose | |
| | escalation study of rAAVhu68.hGALC administered by a single | |
| | ICM injection in pediatric subjects with infantile Krabbe disease. | |
| | Safety and tolerability, pharmacodynamics, and clinical efficacy | |
| | are assessed over 2 years, and all subjects are followed through | |
| | 5 years post-administration of rAAVhu68.hGALC for the long- | |
| | term evaluation of safety and tolerability, pharmacodynamics, | |
| | disease progression, and clinical outcomes. The study design is | |
| | illustrated in FIG. 16. The study consists of a screening phase to | |
| | determine eligibility of each potential subject from approximately | |
| | Day −35 to Day −1. After confirmation of subject eligibility and | |
| | parent's/guardian's willingness to have their child participate in the | |
| | study, the subject undergoes baseline assessments, which include | |
| | brain magnetic resonance imaging (MRI), lumbar puncture (LP) | |
| | for cerebrospinal fluid (CSF) collection, blood draw, urine | |
| | collection, vitals, ECG, a physical exam, a neurological exam and | |
| | clinical assessments. Baseline assessments occur on Days −1 and | |
| | Day 0 and eligibility are reconfirmed at baseline prior to | |
| | administration of rAAVhu68.hGALC. During the treatment phase, | |
| | subjects are admitted to the hospital on the morning of Day 0. | |
| | Subjects receive a single ICM dose of rAAVhu68.hGALC on Day | |
| | 0 and remain in the hospital for at least 24 hours after dosing for | |
| | observation. Subsequent study visits occur at Day 7, Day 14, | |
| | Day 30, 3 months, and 6 months after dosing, followed by every 6 | |
| | months for the first 2 years after dosing as outlined in FIG. 18A- | |
| | 18C. LTFU visits occur for an additional 3 years at a frequency of | |
| | every 12 months, through 5 years post-dosing. | |
| | The study consists of the following three cohorts administered | |
| | rAAVhu68.hGALC as a single ICM injection: | |
| | Cohort 1 (Low Dose): Three eligible subjects (subjects #1 | |
| | to #3) are sequentially enrolled and administered the low | |
| | dose of rAAVhu68.hGALC with a 4 week safety | |
| | observation period between the first and second subject. If | |
| | no safety review triggers (SRTs) are observed, all available | |
| | safety data are evaluated by a safety board 4 weeks after | |
| | the third subject in Cohort 1 is administered | |
| | rAAVhu68.hGALC. | |
| | Cohort 2 (High Dose): If the decision is made to proceed, | |
| | three eligible subjects (Subjects #4 to #6) are sequentially | |
| | enrolled and administered the high dose of | |
| | rAAVhu68.hGALC with a 4 week safety observation | |
| | period between the fourth and fifth subject. If no SRTs are | |
| | observed, the safety board evaluates all available safety | |
| | data, including safety data from subjects in Cohort 1, | |
| | 4 weeks after the third subject Cohort 2 is administered | |
| | rAAVhu68.hGALC. | |
| | Cohort 3 (MTD): Pending a positive recommendation by | |
| | the safety board, 6 additional subjects (Subjects #7-#12) | |
| | are enrolled and administered a single ICM dose | |
| | rAAVhu68.hGALC at the MTD. Dosing for subjects in this | |
| | cohort is not staggered with a 4 week safety observation | |
| | period between each subject, and a safety board review is | |
| | not required following dosing of the first three subjects in | |
| | this cohort. | |
| | Cumulatively, we anticipate a total enrollment of 9 subjects in | |
| | either the high- or low-dose cohort, and 12 subjects in total (across | |
| | all doses). | |
| Inclusion Criteria | 1. | Older than 1 month of age at dosing |
| | 2. | Diagnosis of Krabbe disease confirmed by low GALC |
| | | activity, high psychosine levels, and genetic documentation of |
| | | homozygous or compound heterozygous GALC deletions or |
| | | mutations |
| | 3. | Subjects enrolling in Cohort 1 or Cohort 2 have documented |
| | | symptom onset before 9 months of age |
| | 4. | Subjects enrolling in Cohort 3 must have one of the following: |
| | a) | documented symptom onset before 9 months of age, OR |
| | b) | be presymptomatic AND have a sibling with a confirmed |
| | | diagnosis of Krabbe disease who had symptom onset before |
| | | 9 months of age, OR have been identified through NBS and |
| | | have a diagnosis of IKD based on the Consensus Guidelines |
| | | for NBS, Diagnosis and Treatment (Kwon J. M., et al. (2018) |
| | | Orphanet J Rare Dis. 13(1): 30) |
| Exclusion Criteria | 1. | Subjects in Cohort 1 and Cohort 2: Prior HSCT with evidence |
| | | of residual cells of donor origin |
| | 2. | Subjects end stage disease presenting with any of the |
| | | following symptoms: |
| | | deafness |
| | | blindness |
| | | severe weakness with loss of primitive reflexes |
| | 3. | Subjects presenting with more than one of the following signs: |
| | | abnormal pupillary reflexes |
| | | jerky eye movement |
| | | visual tracking difficulties |
| | 4. | Any clinically significant neurocognitive deficit not |
| | | attributable to Krabbe disease or a secondary cause that may in |
| | | the opinion of the investigator confound interpretation of study |
| | | results. |
| | 5. | Patients with a positive test result for human |
| | | immunodeficiency virus (HIV) or Hepatitis C |
| | 6. | Any condition (e.g., history of any disease, evidence of any |
| | | current disease, any finding upon physical examination, or any |
| | | laboratory abnormality) that, in the opinion of the investigator, |
| | | would put the subject at undue risk or would interfere with |
| | | evaluation of the investigational product or interpretation of |
| | | subject safety or study results. |
| | 7. | Any contraindication to ICM administration procedure, |
| | | including contraindications to fluoroscopic imaging. |
| | 8. | Any contraindication to MRI or lumbar puncture. |
| | 9. | Enrollment in any other clinical study with an investigational |
| | | product within 4 weeks prior to Screening or within 5 half-lives |
| | | of the investigational product used in that clinical study, |
| | | whichever is longer |

| | |
|---|---|
| Investigational | rAAVhu68.hGALC |
| Product | |
| Route of | rAAVhu68.hGALC is administered as a single dose to hospitalized |
| Administration | subjects on Day 0 via CT-guided sub-occipital injection into the |
| and Procedure | cisterna magna. |
| | On Day 0, a syringe containing 5.6 mL of rAAVhu68.hGALC at |
| | the appropriate titer is prepared by the Investigational Pharmacy |
| | associated with the study and delivered to the procedure room. |
| | Prior to study drug administration, the subject is anesthetized, |
| | intubated, and the injection site is prepped and draped using sterile |
| | technique. An LP is performed to remove a predetermined volume |
| | of CSF, after which iodinated contrast is IT injected to aid in |
| | visualization of relevant anatomy of the cisterna magna. IV |
| | contrast may be administered prior to or during needle insertion as |
| | an alternative to the IT contrast. The decision to use IV or IT |
| | contrast is at the discretion of the interventionalist performing the |
| | procedure. A spinal needle (22-25 G) is advanced into the cisterna |
| | magna under fluoroscopic guidance. A larger introducer needle |
| | may be used to assist with needle placement. After confirmation of |
| | needle placement, the extension set is attached to the spinal needle |
| | and allowed to fill with CSF. At the discretion of the |
| | interventionalist, a syringe containing contrast material may be |
| | connected to the extension set and a small amount injected to |
| | confirm needle placement in the cisterna magna. After the needle |
| | placement is confirmed, the syringe containing rAAVhu68.hGALC |
| | is connected to the extension set. The syringe contents are slowly |
| | injected over 1-2 minutes, delivering a volume of 5.0 mL. |
| Safety | Safety assessments, including collection of adverse events (AEs) |
| Assessments | and serious adverse events (SAEs), physical and neurologic |
| | examinations, vital signs, clinical laboratory tests (serum |
| | chemistry, hematology, coagulation, LFTs, urinalysis), ECGs, |
| | nerve conduction studies and CSF cytology and chemistry (cell |
| | counts, protein, glucose) is performed at the times indicated in the |
| | study schedule (FIG. 18A-18C). |
| | The Investigator has primary responsibility for the ongoing |
| | medical review of safety data (AEs, SAEs, laboratory data, etc.) |
| | throughout the study and prior to enrollment of each subject during |
| | the dose escalation phase. A safety board reviews safety data at |
| | specified intervals throughout the study and make |
| | recommendations to the Sponsor regarding further conduct of the |
| | study. Safety evaluations after the first three subjects in Cohort 1 |
| | and after the first three subjects in Cohort 2 are conducted as |
| | described in FIG. 17. |

### Statistical Methods

No statistical comparisons are planned for safety evaluations; all results are descriptive only. Data are listed and summary tables are produced.

Statistical comparisons are performed for secondary and exploratory endpoints. Measurements at each time point are compared to baseline values for each subject, as well as data from age matched healthy controls and natural history data from Krabbe disease patients with comparable cohort characteristics where available for each endpoint.

All data are presented in subject data listings. Categorical variables are summarized using frequencies and percentages, and continuous variables are summarized using descriptive statistics (number of non-missing observations, mean, SD, median, minimum, and maximum). Graphical displays are presented as appropriate.

### Population Rationale

### Study Population—Pediatric Patients

The FIH focuses on infantile subjects with symptom onset before 9 months of age, who represent the population with the highest unmet need as HSCT is not indicated for these patients. Furthermore, these patients have a singularly devastating disease course with rapid and highly predictable decline that is homogeneous in the presentation of both motor and cognitive impairment (Bascou N., et al. (2018) Orphanet J Rare Dis. 13(1):126). In fact, patients presenting with symptoms before 9 months of age have a disease course that resembles early infantile Krabbe Disease, with rapid and severe cognitive and motor impairment progression, and failure to gain any functional skills following initial signs and symptoms of disease. The majority of these patients is expected to die within the first few years of life (2 year survival ranges from 26-50% (Duffner P. K., et al. (2011) Pediatr Neurol. 45(3):141-8; Beltran-Quintero M. L., et al. (2019) Orphanet J Rare Dis. 14(1):46). The phenotype of infants with onset between 9 and 12 months of age is more variable, with some exhibiting the severe early infantile Krabbe Disease phenotype, while others have a less severe disease presentation with (near) normal cognition and markedly better adaptive and fine motor skills, which makes it difficult to predict the phenotype of a newly diagnosed patient with onset between 9 and 12 months (Bascou N., et al. (2018) Orphanet J Rare Dis. 13(1):126). Consequently, the population is restricted to subjects with symptom onset <9 months of age whose predictable and rapid decline supports a robust study design and evaluation of functional outcomes within a reasonable follow up period. For this group, treatment is expected to stabilize disease progression and prevent loss of skills such as acquired developmental and motor milestones, prolong survival, delay or prevent development of seizures.

Despite a shared underlying pathophysiology, the adult Krabbe phenotype and disease course is notably milder from devastating infantile Krabbe Disease form so demonstration of disease stabilization in adults would not provide reason to believe for the therapy in infantile Krabbe Disease. Importantly, adult Krabbe disease onset is highly variable, and progression is slower and more variable with decline occurring over many years to decades (Jardim L. B., et al. (1999) Arch Neurol. 56(8):1014-7; Debs R., et al. (2013) J Inherit Metab Dis. 36(5):859-68). It would be very challenging to design a clinical trial that could unequivocally demonstrate efficacy of the investigational therapy in the context of a protracted natural course. The fact that HSCT provides a treatment option able to stabilize or even improve the disease manifestations is another important consideration (Sharp M. E., et al. (2013) JIMD Rep. 10:57-9; Laule C., et al. (2018) Journal of Neuroimaging. 28(3):252-255). Finally, NBS has not been widely adopted in the US and is not available in Europe, and the ambiguous, non-specific clinical presentation means that adult Krabbe disease continues to be underdiagnosed and thus access to such patients remains exceedingly rare (Wasserstein M. P., et al. (2016) Genet Med. 18(12):1235-1243).

Study Population—Exclusion of Subjects with Severe Disease

Given the nature of Krabbe disease with CNS injury thought to be largely irreversible and the very rapid disease progression in the infantile population, rAAVhu68.hGALC is expected to confer the greatest potential for benefit in patients with no or mild to moderate disease that do not exhibit signs that are uniquely associated with the latter stages of disease, including deafness, blindness, severe weakness with loss of primitive reflexes (Escolar M. L., et al. (2006) Pediatrics. 118(3):e879-89). Additionally, abnormal pupillary reflexes, jerky eye movement, or visual tracking difficulties are more common in very advanced disease than in patients with moderate signs and symptoms, and are not typically observed in the early disease stages (Escolar M. L., et al. (2006) Pediatrics. 118(3):e879-89). Therefore, evidence of more than one of these signs are considered an indicator of advanced disease and result in exclusion from the trial. Due to the severe disability, these patients would be unlikely to gain substantial benefit from the therapy beyond stabilization of disease at a low level of clinical function are excluded, the benefit/risk profile would not be favorable, and they would exhibit floor effects on various clinical and instrumental assessments that would preclude evaluation of the efficacy of rAAVhu68.hGALC. This population may also present with a higher risk for non-treatment-related safety concerns due to the advanced state of disease sequelae and are excluded from this trial.

Patients with clinical seizures are not excluded from the trial, unless in the opinion of the Investigator the child has other signs of advanced disease and would be unlikely to benefit from treatment. This is because 1) seizures are not uniquely associated with advanced disease and 2) seizures are an endpoint in the trial and excluding patients with seizures might bias the study towards a population that is less prone to experience seizures.

### Study Population—Inclusion of Presymptomatic Subjects

Presymptomatic infantile Krabbe Disease patients are excluded from the dose escalation portion of the study (Cohort 1 and Cohort 2) in which rAAVhu68.hGALC alone is evaluated. For these patients, at least in the US, HSCT is considered a therapeutic option and the treatment of choice, even if it only serves to delay disease progression. The prevailing US KOL opinion is that testing an unproven investigational therapy would be considered unethical in this population because the exceedingly narrow therapeutic window would effectively deprive the patient of access to a treatment shown to provide at least partial benefit (i.e., should gene therapy prove unsuccessful there would unlikely be time to “rescue” with HSCT). Thus, rAAVhu68.hGALC should be reserved for patients with the clearest unmet need (i.e., infantile Krabbe disease patients with signs and symptoms who are not eligible for HSCT).

Our preclinical studies confirm superior efficacy of a gene therapy and HSCT combined over either approach alone, thus the combination therapy of HSCT and rAAVhu68.hGALC is evaluated as an expansion cohort in the FIH study (Cohort 3). Both symptomatic and pre-symptomatic patients who meet the criteria outlined in this document are eligible for enrollment as this cohort evaluates the safety and efficacy of rAAVhu68.hGALC in the context of HSCT and only progresses if the preclinical data provide a reason to believe for improved efficacy over HSCT alone.

### Study Population—Justification of the Lower Age Limit

Given that symptom onset can occur perinatally, or even in utero, treatment occurs as early as possible to maximize potential benefit, thus the minimum age of the study was selected as 1 month old at dosing as current consensus guidelines recommend HSCT before 1 month of age in eligible patients (Kwon J. M., et al. (2018) Orphanet J Rare Dis. 13(1):30). Requiring subjects to be 1 month or older allows subjects and families to consider other forms of standard-of-care treatment prior to their eligibility for this trial.

Another consideration in selecting the lower age limit is to ensure that the treatment, and specifically the ICM procedure can be safely carried out in such a young patient. After careful review of imaging scans from infants as young as 1 or 2 weeks of age, an expert interventional radiologist at the University of Pennsylvania confirmed that there is no specific anatomical concern with performing CT guided ICM administration in a 1-month-old infant, provided the rationale for treatment is supported.

### Endpoints

In addition to measuring safety and tolerability as primary endpoints, secondary and exploratory pharmacodynamic and efficacy endpoints were chosen for this study based on the current literature and in consultation with leading clinicians specializing in Krabbe disease. These endpoints are anticipated to demonstrate meaningful functional and clinical outcomes in this population. Endpoints are measured at 30 days, 90 days and 6 months, and then every 6 months during the 2-year short-term follow-up period, except for those that require sedation and/or a lumbar puncture, as presented in FIG. 18A-18C. During the long-term extension phase, measurement frequency decreases to once every 12 months. These time points were selected to facilitate thorough assessment of the safety and tolerability of rAAVhu68.hGALC. The early time points and 6 monthly interval were also selected in consideration of the rapid rate of disease progression in untreated infantile Krabbe patients. This allows for thorough evaluation of pharmacodynamics and clinical efficacy measures in treated subjects over a period of follow up for which untreated comparator data exist. Subjects continue to be monitored for safety and efficacy for a total of 5 years after rAAVhu68.hGALC administration, in accordance with the draft “FDA Guidance for Industry: Long Term Follow-Up After Administration of Human Gene Therapy Products” (July 2018).

### Disease Progression and Clinical Outcomes

In view of the rapid and homogeneous rate of disease progression in the infantile population (Duffner P. K., et al. (2011) Pediatr Neurol. 45(3):141-8; Bascou N., et al. (2018) Orphanet J Rare Dis. 13(1):126) a 2 year follow up for primary outcomes evaluation is considered sufficient to evaluate the impact of rAAVhu68.hGALC over time. In addition, the LTFU to 5 years post-treatment is very informative for assessing long-term outcomes and if the treatment is effective in prolonging survival and stabilizing patients at a level of function similar or superior to the outcomes observed in presymptomatic patients after HSCT.

Administration of rAAVhu68.hGALC stabilizes disease progression as measured by survival, preventing loss of developmental and motor milestone potentially supporting acquisition of new milestones, onset and frequency of seizures. Death typically occurs in the first 3 years of life for a majority patients diagnosed with early infantile Krabbe disease, with median mortality extending to 5 years in the late infantile population which incorporates patients with symptom onset from 7-12 months (Duffner P. K., et al. (2012) Pediatr Neurol. 46(5):298-306). By limiting the inclusion criteria to patients with onset on or before 9 months of age, the population has more severe, early infantile-like phenotype and disease course (Bascou N., et al. (2018) Orphanet J Rare Dis. 13(1):126). Given the rapid decline seen in cases of untreated infantile Krabbe disease, treatment with rAAVhu68.hGALC extends life expectancy during the follow-up periods. Motor milestone development depends on the age and stage of disease at the time of subject enrollment (Bascou N., et al. (2018) Orphanet J Rare Dis. 13(1):126; Beltran-Quintero M. L., et al. (2019) Orphanet J Rare Dis. 14(1):46). Given the severity of disease in the target population, subjects may have achieved motor skills by enrollment, developed and subsequently lost other motor milestones, or not yet shown signs of motor milestone development. Assessments therefore track age-at-achievement and age-at-loss for all milestones. Motor milestone achievement is defined for six gross milestones based on the World Health Organization (WHO) criteria outlined in Table 11.

TABLE 11
WHO Performance Criteria for Gross Motor Milestones

| Gross Motor | Multicenter Growth Reference |
|---|---|
| Milestone | Study Performance Criteria |
| Sitting without | Child sits up straight with the head erect for at least 10 seconds. |
| support | Child does not use arms or hands to balance body or support |
| | position. |
| Hands-and-knees | Child alternately moves forward or backward on hands and |
| crawling | knees. The stomach does not touch the supporting surface. |
| | There are continuous and consecutive movements, at least three |
| | in a row. |
| Standing with | Child stands in upright position on both feet, holding onto a |
| assistance | stable object (e.g., furniture) with both hands without leaning |
| | on it. The body does not ouch the stable object, and the legs |
| | support most of the body weight. Child thus stands with |
| | assistance for at least 10 seconds. |
| Walking with | Child is in upright position with the back straight. Child makes |
| assistance | sideways or forward steps by holding on a stable objects (e.g., |
| | furniture) with one of both hands. One leg moves forward while |
| | the other supports part of the body weight. Child takes at least |
| | five steps in this manner. |
| Standing alone | Childs stands in upright position on both feed (not on the toes) |
| | with the back straight. The legs support 100% of the child's |
| | weigh. There is no contact with a person or objects. Child |
| | stands alone for at least 10 seconds. |
| Walking alone | Child takes at least five steps independently in upright position |
| | with the back straight. One leg moves forward while the other |
| | supports most of the body weight. There is no contact with a |
| | person or object. |

Adapted from (Wijnhoven T. M., et al. (2004) Food Nutr Bull. 25(1 Suppl): S37-45).
Abbreviations: WHO, World Health Organization.

Given that subjects with infantile Krabbe disease can develop symptoms within the first weeks or months of life, and acquisition of the first WHO motor milestone (sitting without support) typically does not manifest before 4 months of age (median: 5.9 months of age), this endpoint may lack sensitivity to evaluate the extent of therapeutic benefit, especially in subjects who had more overt symptoms at the time of treatment. For this reason, assessment of age appropriate developmental milestones that can be applied to infants are also included (Sharp M. E., et al. (2013) JIMD Rep. 10:57-9). One short-coming is that the published tool is intended for use by clinicians and parents, and organizes skills around the typical age of milestone acquisition without referencing normal ranges. However, the data may be informative to summarize retention, acquisition, or loss of developmental milestones over time, relative to untreated children with infantile Krabbe disease or the typical time of acquisition in neurotypical children.

While seizures are not a presenting symptom for the infantile population, approximately 30-60% of infantile patients will eventually develop seizures in the later stages of the disease (Duffner P. K., et al. (2011) Pediatr Neurol. 45(3):141-8). The delayed onset of seizure activity enables us to determine if treatment with rAAVhu68.hGALC can either prevent or delay onset of seizures in this population, or decrease the frequency of seizure events. Parents are asked to keep seizure diaries which track onset, frequency, length and type of seizure. These entries are discussed with and interpreted by the clinician at each visit.

As exploratory measures, clinical scales are used to quantify the effects of rAAVhu68.hGALC on development and changes in adaptive behaviors, cognition, language, motor function, and health-related quality of life. Each measure proposed has been used either in the Krabbe population or in a related population.

The scale and relevant domains are briefly described below:
Bayley Scales of Infant and Toddler Development (Edition III): Assesses development of infant and toddlers across five domains: cognitive, language, motor, social-emotional, and adaptive behavior. All domains are assessed in the trial.
Vineland Adaptive Behavior Scales (Edition III): Assesses adaptive behavior from birth through adulthood (0-90 years) across five domains: communication, daily living skills, socialization, motor skills, and maladaptive behavior. Improvements from v2 to v3 incorporate questions to enable a better understanding of developmental disabilities.
Peabody Developmental Motor Scales-Second Edition: Measures interrelated motor function from birth to children five years of age. Assessments focus on six domains: reflexes, stationary, locomotion, object manipulation, grasping, and visual-motor integration
Infant Toddler Quality of Life Questionnaire (ITQOL): Parent-reported measure of health-related quality of life designed for infants 2 months of age up to toddlers 5 years of age.
Mullen Scales of Early Learning: Assesses language, motor, and perceptual abilities in infants and toddlers up to 68 months of age.

### Disease Biomarkers

To assess the effect of rAAVhu68.hGALC on disease pathology, changes in myelination, functional outcomes related to myelination, and potential disease biomarkers are measured. As the primary hallmark of disease, central and peripheral demyelination slow or cease in progression with rAAVhu68.hGALC administration. Central demyelination is tracked by diffusion-tensor magnetic resonance imaging (DT-MRI) anisotropy measurements of white matter regions and fiber tracking of corticospinal motors tracts, changes in which are indicators of disease state and progression (McGraw P., et al. (2005) Radiology. 236(1):221-30; Escolar M. L., et al. (2009) AJNR Am J Neuroradiol. 30(5):1017-21). Peripheral demyelination is measured indirectly via nerve conduction velocity (NCV) studies on the motor nerves (deep peroneal, tibial, and ulnar nerves) and sensory nerves (sural, and median nerves) to monitor for fluctuations indicative of a change in biologically active myelin (i.e., F-wave and distal latencies, amplitude or presence or absence of a response).

Development of visual impairment is common in early infantile Krabbe, with 61.2% of the population developing vision loss at some point in the disease according to one study (Duffner P. K., et al. (2011) Pediatr Neurol. 45(3):141-8). Similar to seizures, vision loss is not a common presenting symptom. This offers the opportunity to assess the ability of rAAVhu68.hGALC to delay or prevent vision loss for those subjects that have not developed significant vision loss prior to treatment. Measurement of visual evoked potentials (VEPs) is therefore used to objectively measure responses to visual stimuli as an indicator of central visual impairment or loss. Hearing loss is also common during disease progression and early indications of auditory abnormalities are measured via brainstem auditory evoked response (BAER) testing.

GALC is responsible for the hydrolysis of psychosine. Deficiency of GALC in Krabbe disease results in the accumulation of psychosine both centrally and peripherally. Increased levels of psychosine have been proposed as an indicator of Krabbe disease (Escolar M. L., et al. (2017) Mol Genet Metab. 121(3):271-278). While there is evidence to support its use in detection of early and severe cases of infantile Krabbe, interpretation of fluctuations in psychosine levels over time, following treatment may be difficult, as psychosine levels may also decline in late-stage disease. Thus, evidence of decline in psychosine levels alone would not be sufficient evidence of a treatment effect, unless it was accompanied by clinical disease stabilization.

### Sequence Listing Free Text

The following information is provided for sequences containing free text under numeric identifier <223>.

| SEQ ID NO: | |
|---|---|
| (containing free | |
| text) | Free text under <223> |
| 1 | <223> AAVhu68 vp1 capsid |
| 2 | <223> Synthetic Construct |
| 3 | <223> AAV9 VP1 capsid |
| | <220> |
| | <221> CDS |
| | <222> (1) . . . (2208) |
| | <223> AAV9 VP1 Capsid |
| 4 | <223> Synthetic Construct |
| 5 | <223> Human GALC coding sequence |
| | <220> |
| | <221> sig_peptide |
| | <222> (1) . . . (126) |
| | <220> |
| | <221> CDS |
| | <222> (1) . . . (2058) |
| 6 | <223> Synthetic Construct |
| 7 | <223> Engineered canine GALC |
| | <220> |
| | <221> CDS |
| | <222> (1) . . . (2007) |
| 8 | <223> Synthetic Construct |
| 9 | <223> Engineered human GALC coding sequence |
| | <220> |
| | <221> CDS |
| | <222> (1) . . . (2055) |
| | <220> |
| | <221> sig_peptide |
| | <222> (1) . . . (126) |
| | <220> |
| | <221> mat |
| | <222> (108) . . . (2055) |
| 10 | <223> Synthetic Construct |
| 11 | <223> AAV2 - 5′ ITR |
| 12 | <223> CMV IE promoter |
| 13 | <223> CB promoter |
| 14 | <223> CB7 promoter |
| 15 | <223> chimeric intron |
| 16 | <223> Rabbit globin polyA |
| 17 | <223> AAV2 - 3′ ITR |
| 18 | <223> Vector genome with canine GALC |
| 19 | <223> CB7.CI.hGALC.rBG |
| | <220> |
| | <221> misc_feature |
| | <222> (1) . . . (130) |
| | <223> 5′ ITR |
| | <220> |
| | <221> misc_feature |
| | <222> (198) . . . (579) |
| | <223> CMV IE Enhancer |
| | <220> |
| | <221> misc_feature |
| | <222> (582) . . . (863) |
| | <223> CB promoter |
| | <220> |
| | <221> misc_feature |
| | <222> (836) . . . (839) |
| | <223> TATA |
| | <220> |
| | <221> misc_feature |
| | <222> (958) . . . (1930) |
| | <223> chimeric intron |
| | <220> |
| | <221> misc_feature |
| | <222> (1948) . . . (4002) |
| | <223> hGALCco |
| | <220> |
| | <221> misc_feature |
| | <222> (4042) . . . (4168) |
| | <223> Rabbit globin poly A |
| | <220> |
| | <221> misc_feature |
| | <222> (4257) . . . (4386) |
| | <223> 3′ ITR |
| 20 | <223> AAV1 VP1 gene |
| | <220> |
| | <221> CDS |
| | <222> (1) . . . (2208) |
| 21 | <223> Synthetic Construct |
| 22 | <223> AAV5 capsid VP1 gene |
| | <220> |
| | <221> CDS |
| | <222> (1) . . . (2172) |
| 23 | <223> Synthetic Construct |
| 24 | <223> AAV3B VP1 Capsid |
| 25 | <223> UbC promoter |
| 26 | <223> SV40 late polyA |
| 27 | <223> EF-1a promoter |

All documents cited in this specification are incorporated herein by reference, as are the sequences and the text of the Sequence Listing (labeled “18-8584PCT_ST25.txt”) filed herewith. U.S. Provisional Patent Application No. 62/810,708, filed Feb. 26, 2019, U.S. Provisional Patent Application No. 62/817,482, filed Mar. 12, 2019, U.S. Provisional Patent Application No. 62/877,707, filed Jul. 23, 2019, and U.S. Provisional Patent Application No. 62/916,652, filed Oct. 17, 2019, are incorporated by reference in their entireties, together with their sequence listings. While the invention has been described with reference to particular embodiments, it will be appreciated that modifications can be made without departing from the spirit of the invention. Such modifications are intended to fall within the scope of the appended claims.

1. A composition comprising a recombinant adeno-associated virus (rAAV), said rAAV comprising:
(a) an AAV capsid which targets cells in the central nervous system; and
(b) a vector genome comprising (i) a galactosylceramidase coding sequence encoding a signal peptide and at least a mature human galactosylceramidase protein having the amino acid sequence of aa 43 to 685 of SEQ ID NO: 10 under the control of regulatory sequences which direct expression of the protein, and (ii) AAV inverted terminal repeats necessary for packaging the vector genome in the AAV capsid, wherein the vector genome is packaged in the AAV capsid.

2. The composition according to claim 1, wherein the AAV capsid is an AAVhu68 capsid.

3. The composition according to claim 1, wherein the coding sequence encodes the full-length human galactosylceramidase signal peptide and mature human galactosylceramidase protein of SEQ ID NO: 10 (amino acids 1 to 685).

4. The composition according to claim 1, wherein the coding sequence has the nucleic acid sequence of an exogenous peptide coding sequence and nucleotides 127 to 2055 of SEQ ID NO: 9 or a sequence 95% to 99.9% identical thereto, or the nucleotide sequence of nucleotides 1 to 2055 of SEQ ID NO: 9 or a sequence 95% to 99.9% identical thereto.

5. The composition according to claim 1, wherein the coding sequence encodes the mature human galactosylceramidase protein of SEQ ID NO: 10 (amino acids 43 to 685) and an exogenous signal peptide suitable for human cells in the central nervous system.

6. The composition according to claim 1, wherein the regulatory sequences comprise: a beta-actin promoter, an intron, and a rabbit globin polyA.

7. The composition according claim 1, wherein the regulatory sequences comprise SEQ ID NO: 13, SEQ ID NO: 15, and/or SEQ ID NO 16.

8-9. (canceled)

10. The composition according to claim 1, wherein the vector genome comprises nt 198 to 4168 of SEQ ID NO: 19.

11. The composition according to claim 1, wherein the vector genome further comprises a 5′ ITR of AAV2, the coding sequence and the regulatory sequences, and a 3′ ITR of AAV2.

12. The composition according to claim 1, wherein the composition further comprises an aqueous liquid suitable for intrathecal delivery to a patient.

13. The composition according to claim 1, wherein the composition comprises an artificial cerebrospinal fluid and a surfactant.

14. The composition according to claim 1, wherein the composition is formulated for intrathecal delivery and comprises 1.4×10¹³ to 4×10¹⁴ GC of the rAAV.

15. The composition according to claim 1, wherein the composition is formulated for intra-cisterna magna delivery and comprises 1.4×10¹³ to 4×10¹⁴ GC of the rAAV.

16-31. (canceled)

32. A plasmid comprising a galactosylceramidase coding sequence encoding a signal peptide and a mature human galactosylceramidase protein having the amino acid sequence of amino acids 43 to 685 of SEQ ID NO: 10.

33. The plasmid of claim 32, wherein the coding sequence has the nucleic acid sequence of SEQ ID NO: 9 or a sequence 95% to 99.9% identical thereto.

34. A method for treating Krabbe disease comprising administering to a patient in need thereof a composition comprising a stock of recombinant adeno-associated virus (rAAV), said rAAV comprising: (a) an AAV capsid which targets cells in the central nervous system; and (b) a vector genome comprising a galactosylceramidase coding sequence encoding a signal peptide and a mature human galactosylceramidase protein having the amino acid sequence of amino acids 43 to 685 of SEQ ID NO: 10 under the control of regulatory sequences which direct expression of the protein, said vector genome further comprising AAV inverted terminal repeats necessary for packaging the vector genome in the AAV capsid, wherein the vector genome is packaged in the AAV capsid.

35-36. (canceled)

37. The method according to claim 34, wherein the patient has early infantile Krabbe disease (EIKD), late infantile Krabbe disease (LIKD), juvenile Krabbe disease (JKD), or adolescent/adult onset Krabbe disease.

38-40. (canceled)

41. The method according to claim 34, wherein the AAV capsid is an AAVhu68 capsid.

42. The method according to claim 34, wherein the coding sequence has an exogenous peptide coding sequence and nucleotides 127 to 2055 of SEQ ID NO: 9 or a sequence 95% to 99.9% identical thereto, or the nucleotide sequence of nucleotides 1 to 2055 of SEQ ID NO: 9 or a sequence 95% to 99.9% identical thereto.

43. The method according to claim 34, wherein the coding sequence encodes the mature human galactosylceramidase protein of amino acids 43 to 685 of SEQ ID NO: 10 and an exogenous signal peptide.

44. (canceled)

45. The method according to claim 34, to wherein the rAAV is administered as a co-therapy to hematopoietic stem cell transplant (HSCT) or bone marrow transplant or the rAAV is administered as a co-therapy to substrate reduction therapy.

46-50. (canceled)

51. The method according to claim 34, wherein the composition is administered as a single dose via a computed tomography-(CT-) guided sub-occipital injection into the cisterna magna (intra-cisterna magna).

52-55. (canceled)

56. A production cell comprising the plasmid according to claim 32.
